(19) United States
(12) Patent Application Publication
(10) Pub. No.: US 2025/0145571 A1
(43) Pub. Date: May 8, 2025

(54) AMINO ACID PRODRUGS OF NICORANDIL

(71) Applicant: Unicycive Therapeutics, Inc., Los Altos, CA (US)

(72) Inventor: Roopa RAI, Los Altos, CA (US)

(21) Appl. No.: 18/898,468

(22) Filed: Sep. 26, 2024

Related U.S. Application Data
(60) Provisional application No. 63/540,587, filed on Sep. 26, 2023.
(60) Provisional application No. 63/565,716, filed on Mar. 15, 2024.

(51) Int. Cl. C07D 213/82; A61K 31/465; C07D 401/12; C07D 405/06

(52) U.S. Cl. CPC C07D 213/82; A61K 31/465; C07D 401/12; C07D 405/06

(57) ABSTRACT

The present disclosure is directed to nicorandil derivative compounds, pharmaceutical compositions thereof, and methods of using the compounds and pharmaceutical compositions for treating diseases or conditions.

### CROSS-REFERENCE TO RELATED APPLICATIONS

This application claims priority to, and the benefit of, U.S. Provisional Patent Application No. 63/540,587, filed Sep. 26, 2023. The contents of the provisional application are herein incorporated by reference in their entireties for all purposes.

### FIELD

The present disclosure is directed to nicorandil derivative compounds, pharmaceutical compositions thereof, and methods of using the compounds and pharmaceutical compositions for treating diseases or conditions.

### BACKGROUND OF THE INVENTION

Kidney diseases, such as chronic kidney disease, acute kidney failure and acute kidney disease, are major health concerns in USA. Approximately 20 million people in the United States are currently affected by chronic kidney disease (CKD), with half a million of these diagnosed with the most severe form, known as end-stage renal disease (ESRD). One of the major causes of chronic and ESRD in the United States is diabetes, which results in approximately 50% of all cases. Apart from diabetes, hypertension and cardiovascular disease remains the leading cause of death for patients with CKD. Acute kidney injury (AKI), acute kidney failure (AKF) and diabetic nephropathy are also debilitating kidney diseases that affect millions of people globally and can eventually progress into CKD. AKI is a relatively common condition in the intensive care unit and occurs in 20% to 30% of critically ill patients, with approximately 6% eventually requiring renal replacement therapy. The development of AKI results in increased mortality, longer hospital stays, and eventually, increased healthcare costs. While in most cases, the epidemiological cause of AKI is multifactorial (e.g., sepsis, ischemia/hypoperfusion), recent studies have shown that nephrotoxic drugs are contributing factors in 19% to 25% of cases of severe acute renal failure in patients with serious underlying diseases. The use of nephrotoxic drugs and resulting acute kidney injury is referred to as drug induced AKI and this is a major problem in patients taking drugs, such as antibiotics, anticancer agents, and anti-infectious drugs.

Treatment options for CKD, AKI or AKF range from Renal Replacement Therapy (RRT), transplant, and radical surgery. Angiotensin Converting Enzyme Inhibitors (ACEI), Angiotensin Receptor Blockers (ARBs), or protein-restricted diet have been used in certain conditions to mitigate the effect of these kidney diseases. However, in the majority of the cases, the damage is irreversible and the patient requires a transplant or dialysis for the remainder of their life. Drugs that are currently being used or being developed for CKD include renin inhibitors (Aliskerin), ACE inhibitors (Captopril, Ramipril and Lisinopril), angiotensin receptor inhibitors (Telmisartan and Losartan), mineralocorticoid antagonists (Spironolactone, Finerenone, CS-3150 and MT-3995), and drugs that target Glomerulus abnormalities (sulodexide, Atrasentan, Abatacept, Bis-T-23, rapamycin, lithium and gamma-secretase inhibitors). Additionally, a number of drugs are currently under clinical development for a variety of pathways and targets that result in treating CKD, for example Canagliflozin, Pyridorin, ASP8232, baricitinib, CCX140, CTP140, GKT137831, VPI-2690B, GS-4997, PF-04634817, and Allopurinol.

Nicorandil is a vasodilatory drug used to treat angina and acute heart failure. It is a nitrate and K⁺ ATP channel agonist. In addition to treating angina, nicorandil may be useful to treat kidney diseases (Lee et al., J. Hypertension 2009, 27, 618-625; Iranirad et al., Cardiology Journal (online) 2017, DOI 10.5603/cj.a2017.0028; Ko et al., Yonsei Med J. 2013, 54(4), 957-964; Tamura et al., Am. J. Physiol Renal Physiol 2012, 303, 339-349; Nawa et al., Int. J. Cardiology 2015, 195, 228-234; Tanabe et al., Am. J. Physiol Renal Physiol 2012, 302, 151-160; and US20110257202). Certain nicorandil derivatives are disclosed in PCT Applications WO 2012/137225 and WO 2020/190890.

There is a need for compositions and methods for treating and preventing a wide variety of diseases, including kidney diseases, that are safe and effective compared to conventional treatments.

### SUMMARY OF THE INVENTION

Described herein are compounds of Formula I.

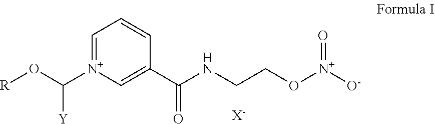

or a stereoisomer thereof;
wherein X⁻ indicates one or more counter ions;
Y is H or C₁-C₄ alkyl;
R is selected from

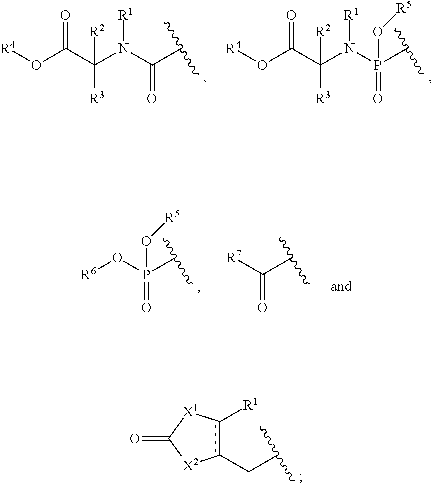

R¹ is H or C₁-C₄ alkyl;
R² is H or C₁-C₄ alkyl;
R³ is H or unsubstituted or substituted C₁-C₄ alkyl; or R¹ and R³ are joined together to form a 4-7 membered substituted or unsubstituted heterocycloalkyl;
R⁴ is H or C₁-C₈ alkyl;
R⁵ and R⁶ are independently selected from hydrogen, C₁-C₈ alkyl, a 6-10 membered substituted or unsubstituted aryl, and a 6-10 membered substituted or unsubstituted heteroaryl;
R⁷ is H or C₁-C₁₂ alkyl;
X¹ and X² are independently selected from NH, O, and S; and
[symbol] is a single or double bond;
wherein when Y is H, R⁷ is not —CH(CH₃)₂, —CH₂CH(CH₃)₂, —C(CH₃)₃, or —CH₂C(CH₃)₃.

In certain embodiments, the compound of Formula (I) is a compound of Formula (I-1):

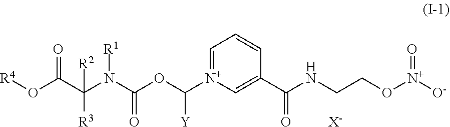

or a stereoisomer thereof.

In certain embodiments, the compound of Formula (I) is a compound of Formula (I-2):

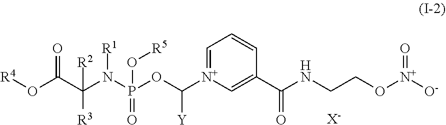

or a stereoisomer thereof.

In certain embodiments, the compound of Formula (I) is a compound of Formula (I-3):

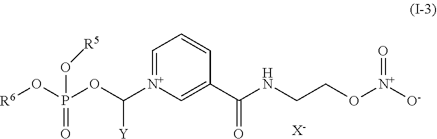

or a stereoisomer thereof.

In certain embodiments, the compound of Formula (I) is a compound of Formula (I-4):

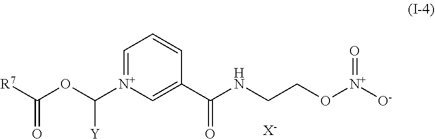

or a stereoisomer thereof.

In certain embodiments, the compound of Formula (I) is a compound of Formula (I-5):

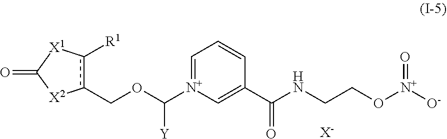

or a stereoisomer thereof.

Also described herein are pharmaceutical formulations comprising a compound of Formula (I), (I-1), (I-1a), (I-1b), (1-2), (I-2a), (I-2b), (1-3), (1-4), or (I-5), and a pharmaceutically acceptable carrier, excipient, and/or diluent. In one embodiment, the pharmaceutical composition is suitable for a route of administration selected from oral, parenteral, buccal, nasal, topical, or rectal. In one embodiment, the pharmaceutical formulation is in a dosage form suitable for oral administration, including liquid and solid dosage forms. In another embodiment, the pharmaceutical formulation is in a dosage form suitable for parenteral administration, including intravenous administration.

Also described herein are methods to treat and prevent diseases and conditions, including, for example, kidney diseases and conditions, comprising administering to a subject in need thereof a compound of Formula (I), (I-1), (I-1a), (I-1b), (I-2), (I-2a), (I-2b), (I-3), (I-4), or (I-5) or a pharmaceutical composition comprising a compound of Formula (I), (I-1), (I-1a), (I-1b), (I-2), (I-2a), (I-2b), (I-3), (I-4), or (I-5). In some embodiments, the disease or condition is chronic kidney disease (CKD), diabetic nephropathy, IgA nephropathy, acute kidney failure, acute tubular necrosis, transplant related ischemia, acute kidney disease and types of AKI such as cisplatin-induced AKI, aminoglycosides-induced AKI, Tenofovir-induced AKI, contrast induced nephropathy, PD-1/PDL-1 inhibitor-induced AKI, AKI after surgery for partial nephrectomy, AKI post kidney transplant, AKI in patients with dialysis, AKI after cardiac surgery, AKI in patients inside ICU, AKI caused by cirrhosis or sepsis and AKI caused by hepatorenal syndrome.

Other objects, features and advantages of the methods and compositions described herein will become apparent from the following detailed description. It should be understood, however, that the detailed description and the specific examples, while indicating specific embodiments, are given by way of illustration only, since various changes and modifications within the spirit and scope of the present disclosure will become apparent to those skilled in the art from this detailed description. The section headings used herein are for organizational purposes only and are not to be construed as limiting the subject matter described. All documents, or portions of documents, cited in the application including, but not limited to, patents, patent applications, articles, books, manuals, and treatises are hereby expressly incorporated by reference in their entirety for any purpose.

### BRIEF DESCRIPTION OF THE DRAWINGS

FIG. 1A depicts test compound concentration and nicorandil formation over time for compound 8* in human plasma.

FIG. 1B depicts test compound concentration and nicorandil formation over time for compound 10 in human plasma.

FIG. 1C depicts test compound concentration and nicorandil formation over time for compound 8* in rat plasma.

FIG. 1D depicts test compound concentration and nicorandil formation over time for compound 10 in rat plasma.

FIG. 2A depicts nicorandil concentration over time in rats following single oral administration of nicorandil prodrugs at 3 mg/kg eq. nicorandil in citrate buffer pH 4.5.

FIG. 2B depicts compound 8* and nicorandil concentrations over time following single oral administration of compound 8* at 3 mg/kg eq. nicorandil in 0.25% carboxymethyl cellulose vehicle.

FIG. 2C depicts nicorandil concentration over time following single oral administration of compound 10 at 3 and 14 mg/kg eq. nicorandil in carboxymethyl cellulose vehicle.

FIG. 2D depicts nicorandil concentration over time following single oral administration of compound 11 at 3 and 14 mg/kg eq. nicorandil in carboxymethyl cellulose vehicle

### DETAILED DESCRIPTION OF THE INVENTION

### Certain Terminology

Unless defined otherwise, all technical and scientific terms used herein have the same meaning as is commonly understood by one of skill in the art to which the claimed subject matter belongs. In the event that there are a plurality of definitions for terms herein, those in this section prevail. Where reference is made to a URL or other such identifier or address, it is understood that such identifiers can change and particular information on the internet can come and go, but equivalent information can be found by searching the internet. Reference thereto evidences the availability and public dissemination of such information.

It is to be understood that the foregoing general description and the following detailed description are exemplary and explanatory only and are not restrictive of any subject matter claimed. In this application, the use of the singular includes the plural unless specifically stated otherwise. It must be noted that, as used in the specification and the appended claims, the singular forms “a,” “an” and “the” include plural referents unless the context clearly dictates otherwise. Use of the term “including” as well as other forms, such as “include”, “includes,” and “included,” is not limiting. Definition of standard chemistry terms may be found in reference works, including Carey and Sundberg “Advanced Organic Chemistry 4th Ed.” Vols. A (2000) and B (2001), Plenum Press, New York. Unless otherwise indicated, conventional methods of mass spectroscopy, NMR, HPLC, protein chemistry, biochemistry, recombinant DNA techniques and pharmacology, within the skill of the art are employed. Unless specific definitions are provided, the nomenclature employed in connection with, and the laboratory procedures and techniques of, analytical chemistry, synthetic organic chemistry, and medicinal and pharmaceutical chemistry described herein are those known in the art. Standard techniques can be used for chemical syntheses, chemical analyses, pharmaceutical preparation, formulation, and delivery, and treatment of patients. Standard techniques can be used for recombinant DNA, oligonucleotide synthesis, and tissue culture and transformation (e.g., electroporation, lipofection). Reactions and purification techniques can be performed e.g., using kits of manufacturer's specifications or as commonly accomplished in the art or as described herein. The foregoing techniques and procedures can be generally performed of conventional methods well known in the art and as described in various general and more specific references that are cited and discussed throughout the present specification.

It is to be understood that the methods and compositions described herein are not limited to the particular methodology, protocols, cell lines, constructs, and reagents described herein and as such may vary. It is also to be understood that the terminology used herein is for the purpose of describing particular embodiments only, and is not intended to limit the scope of the methods and compositions described herein, which will be limited only by the appended claims.

All publications and patents mentioned herein are incorporated herein by reference in their entirety for the purpose of describing and disclosing, for example, the constructs and methodologies that are described in the publications, which might be used in connection with the methods, compositions and compounds described herein. The publications discussed herein are provided solely for their disclosure prior to the filing date of the present application. Nothing herein is to be construed as an admission that the inventors described herein are not entitled to antedate such disclosure by virtue of prior invention or for any other reason.

“Alkyl” refers to a straight or branched hydrocarbon chain radical consisting solely of carbon and hydrogen atoms, containing no unsaturation, having from one to eight carbon atoms (e.g., C₁-C₅ alkyl). In certain embodiments, an alkyl comprises five to eight carbon atoms (e.g., C₅-C₈ alkyl). The alkyl is attached to the rest of the molecule by a single bond, for example, methyl (Me), ethyl (Et), n-propyl (n-pr), 1-methylethyl (iso-propyl or i-Pr), n-butyl (n-Bu), n-pentyl, 1,1-dimethylethyl (t-butyl, or t-Bu), 3-methylhexyl, 2-methylhexyl, and the like. Unless stated otherwise specifically in the specification, an alkyl group is optionally substituted as defined and described below and herein.

“Cycloalkyl” refers to a stable fully saturated non-aromatic monocyclic or polycyclic hydrocarbon radical consisting solely of carbon and hydrogen atoms, which includes fused or bridged ring systems, having from three to fifteen carbon atoms. In certain embodiments, a cycloalkyl comprises three to ten carbon atoms. In some embodiments, a cycloalkyl comprises five to seven carbon atoms. The cycloalkyl is attached to the rest of the molecule by a single bond. Examples of monocyclic cycloalkyls include, e.g., cyclopropyl, cyclobutyl, cyclopentyl, cyclohexyl, cycloheptyl, and cyclooctyl.

“Heterocycloalkyl” refers to a non-aromatic ring wherein one or more atoms forming the ring is a heteroatom. A “heterocycloalkyl” group refers to a cycloalkyl group that includes at least one heteroatom selected from nitrogen, oxygen and sulfur. The radicals may be fused with an aryl or heteroaryl. Heterocycloalkyl rings can be formed by three to seven ring atoms, such as three, four, five, six, or seven atoms. Heterocycloalkyl rings can be optionally substituted. In certain embodiments, heterocycloalkyl contain one or more carbonyl or thiocarbonyl groups such as, for example, oxo- and thio-containing groups. Examples of heterocycloalkyls include, but are not limited to, lactams, lactones, cyclic imides, cyclic thioimides, cyclic carbamates, tetrahydrothiopyran, 4H-pyran, tetrahydropyran, piperidine, 1,3-dioxin, 1,3-dioxane, 1,4-dioxin, 1,4-dioxane, piperazine, 1,3-oxathiane, 1,4-oxathiin, 1,4-oxathiane, tetrahydro-1,4-thiazine, 2H-1,2-oxazine, maleimide, succinimide, barbituric acid, thiobarbituric acid, dioxopiperazine, hydantoin, dihydrouracil, morpholine, trioxane, hexahydro-1,3,5-triazine, tetrahydrothiophene, tetrahydrofuran, pyrroline, pyrrolidine, pyrrolidone, pyrrolidione, pyrazoline, pyrazolidine, imidazoline, imidazolidine, 1,3-dioxole, 1,3-dioxolane, 1,3-dithiole, 1,3-dithiolane, isoxazoline, isoxazolidine, oxazoline, oxazolidine, oxazolidinone, thiazoline, thiazolidine, and 1,3-oxathiolane. Illustrative examples of heterocycloalkyl groups include:

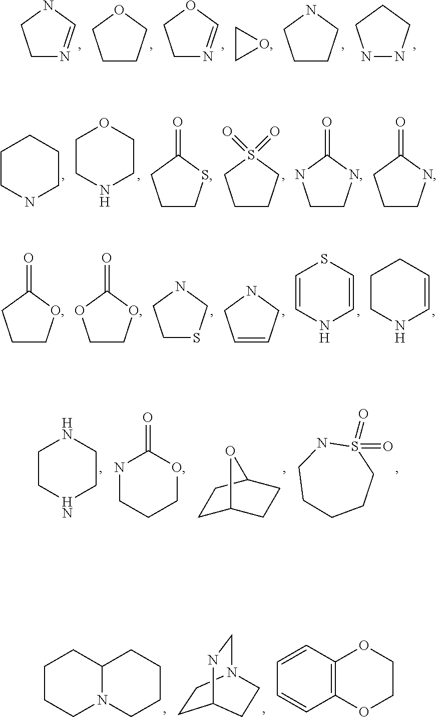

and the like. The term heterocycloalkyl also includes all ring forms of the carbohydrates, including but not limited to the monosaccharides, the disaccharides and the oligosaccharides. Depending on the structure, a heterocycloalkyl group can be a monoradical or a diradical (i.e., a heterocycloalkylene group).

“Aryl” refers to a radical derived from an aromatic monocyclic or multicyclic hydrocarbon ring system by removing a hydrogen atom from a ring carbon atom. The aromatic monocyclic or multicyclic hydrocarbon ring system contains only hydrogen and carbon from six to ten carbon atoms, where at least one of the rings in the ring system is fully unsaturated, i.e., it contains a cyclic, delocalized (4n+2) π-electron system in accordance with the Hückel theory. Aryl groups include, but are not limited to, groups such as phenyl (Ph), fluorenyl, and naphthyl. Unless stated otherwise specifically in the specification, the term “aryl” or the prefix “ar-” (such as in “aralkyl”) is meant to include aryl radicals optionally substituted as defined and described below and herein.

“Heteroaryl” refers to a radical derived from a 3- to 10-membered aromatic ring radical that comprises two to nine carbon atoms and from one to six heteroatoms selected from nitrogen, oxygen and sulfur. As used herein, the heteroaryl radical is a monocyclic, bicyclic, tricyclic or tetracyclic ring system, wherein at least one of the rings in the ring system is fully unsaturated, i.e., it contains a cyclic, delocalized (4n+2) π-electron system in accordance with the Hückel theory. Heteroaryl includes fused or bridged ring systems. The heteroatom(s) in the heteroaryl radical is optionally oxidized. One or more nitrogen atoms, if present, are optionally quaternized. The heteroaryl is attached to the rest of the molecule through any atom of the ring(s). Examples of heteroaryls include, but are not limited to, azepinyl, acridinyl, benzimidazolyl, benzindolyl, 1,3-benzodioxolyl, benzofuranyl, benzooxazolyl, benzo[d]thiazolyl, benzothiadiazolyl, benzo[b][1,4]dioxepinyl, benzo[b][1,4]oxazinyl, 1,4-benzodioxanyl, benzonaphthofuranyl, benzoxazolyl, benzodioxolyl, benzodioxinyl, benzopyranyl, benzopyranonyl, benzofuranyl, benzofuranonyl, benzothienyl (benzothiophenyl), benzothieno[3,2-d]pyrimidinyl, benzotriazolyl, benzo[4,6]imidazo[1,2-a]pyridinyl, carbazolyl, cinnolinyl, cyclopenta[d]pyrimidinyl, 6,7-dihydro-5H-cyclopenta[4,5]thieno[2,3-d]pyrimidinyl, 5,6-dihydrobenzo[h]quinazolinyl, 5,6-dihydrobenzo[h]cinnolinyl, 6,7-dihydro-5H-benzo[6,7]cyclohepta[1,2-c]pyridazinyl, dibenzofuranyl, dibenzothiophenyl, furanyl, furanonyl, furo[3,2-c]pyridinyl, 5,6,7,8,9,10-hexahydrocycloocta[d]pyrimidinyl, 5,6,7,8,9,10-hexahydrocycloocta[d]pyridazinyl, 5,6,7,8,9,10-hexahydrocycloocta[d]pyridinyl, isothiazolyl, imidazolyl, indazolyl, indolyl, indazolyl, isoindolyl, indolinyl, isoindolinyl, isoquinolyl, indolizinyl, isoxazolyl, 5,8-methano-5,6,7,8-tetrahydroquinazolinyl, naphthyridinyl, 1,6-naphthyridinonyl, oxadiazolyl, 2-oxoazepinyl, oxazolyl, oxiranyl, 5,6,6a,7,8,9,10,10a-octahydrobenzo[h]quinazolinyl, 1-phenyl-1H-pyrrolyl, phenazinyl, phenothiazinyl, phenoxazinyl, phthalazinyl, pteridinyl, purinyl, pyrrolyl, pyrazolyl, pyrazolo[3,4-d]pyrimidinyl, pyridinyl, pyrido[3,2-d]pyrimidinyl, pyrido[3,4-d]pyrimidinyl, pyrazinyl, pyrimidinyl, pyridazinyl, pyrrolyl, quinazolinyl, quinoxalinyl, quinolinyl, isoquinolinyl, tetrahydroquinolinyl, 5,6,7,8-tetrahydroquinazolinyl, 5,6,7,8-tetrahydrobenzo[4,5]thieno[2,3-d]pyrimidinyl, 6,7,8,9-tetrahydro-5H-cyclohepta[4,5]thieno[2,3-d]pyrimidinyl, 5,6,7,8-tetrahydropyrido[4,5-c]pyridazinyl, thiazolyl, thiadiazolyl, triazolyl, tetrazolyl, triazinyl, thieno[2,3-d]pyrimidinyl, thieno[3,2-d]pyrimidinyl, thieno[2,3-c]pridinyl, and thiophenyl (i.e. thienyl). Unless stated otherwise specifically in the specification, the term “heteroaryl” is meant to include heteroaryl radicals as defined above which are optionally substituted as defined and described below and herein.

“Heteroalkyl” is an optionally substituted alkyl radical in which one or more skeletal chain atoms is a heteroatom, e.g., oxygen, nitrogen, sulfur, silicon, phosphorus or combinations thereof. The heteroatom(s) may be placed at any interior position of the heteroalkyl group or at the position at which the heteroalkyl group is attached to the remainder of the molecule. Examples include, but are not limited to, —CH₂—O—CH₃, —CH₂—CH₂—O—CH₃, —CH₂—NH—CH₃, —CH₂—CH₂—NH—CH₃, —CH₂—N(CH₃)—CH₃, —CH₂—CH₂—NH—CH₃, —CH₂—CH₂—N(CH₃)—CH₃, —CH₂—S—CH₂—CH₃, —CH₂—CH₂, —S(O)—CH₃, —CH₂—CH₂—S(O)₂—CH₃, —CH═CHO—CH₃, —Si(CH₃)₃, —CH₂—CH═N—OCH₃, and —CH═CH—N(CH₃)—CH₃. In addition, up to two heteroatoms may be consecutive, such as, by way of example, —CH₂—NH—OCH₃ and —CH₂—O—Si(CH₃)₃.

“Hydroxy” refers to —OH.

“Amide” is a chemical moiety with the formula —C(O)NHR or —NHC(O)R, where R is selected from among alkyl, cycloalkyl, aryl, heteroaryl (bonded through a ring carbon) and heteroalicyclic (bonded through a ring carbon).

“Amino” or “amine” is —NH₂.

“Guanidine” or “guanidino” is —NHC(═NH)NH₂.

“Carboxylic acid” is —COOH.

As described herein, compounds of the invention may be “optionally substituted”. In general, the term “substituted,” whether preceded by the term “optionally” or not, means that one or more hydrogens of a designated moiety are replaced with a suitable substituent. Unless otherwise indicated, an “optionally substituted” group may have a suitable substituent at each substitutable position of the group, and when more than one position in any given structure may be substituted with more than one substituent selected from a specified group, the substituent may be either the same or different at every position. Combinations of substituents envisioned by this invention are preferably those that result in the formation of stable or chemically feasible compounds. The term “stable,” as used herein, refers to compounds that are not substantially altered when subjected to conditions to allow for their production, detection, and, in certain embodiments, their recovery, purification, and use for one or more of the purposes disclosed herein.

Suitable monovalent substituents on a substitutable carbon atom of an “optionally substituted” or “substituted” group are independently halogen; —(CH₂)₀₋₄R^{∘}; C₃₋₇ cycloalkyl that may be substituted with R^{∘}, —(CH₂)₀₋₄OR^{∘}; —O(CH₂)₀₋₄R^{∘}, —O—(CH₂)₀₋₄C(O)OR^{∘}; —(CH₂)₀₋₄CH(OR^{∘})₂; —(CH₂)₀₋₄SR^{∘}; —(CH₂)₀₋₄Ph, which may be substituted with R^{∘}; —(CH₂)₀₋₄O(CH₂)₀₋₁Ph that may be substituted with R^{∘}; —CH═CHPh, which may be substituted with R^{∘}; —(CH₂)₀₋₄O(CH₂)₀₋₁-pyridyl which may be substituted with R^{∘}; —NO₂; —CN; —N₃; —(CH₂)₀₋₄N(R^{∘})₂; —(CH₂)₀₋₄N(R^{∘})C(O)R^{∘}; —N(R^{∘})C(S)R^{∘}; —(CH₂)₀₋₄N(R^{∘})C(O)NR⁰²; —N(R^{∘})C(S)NR^{∘}₂; —(CH₂)₀₋₄N(R^{∘})C(O)OR^{∘}; —N(R^{∘})N(R^{∘})C(O)R^{∘}; —N(R^{∘})N(R^{∘})C(O)NR^{∘}₂; —N(R^{∘})N(R^{∘})C(O)OR^{∘}; —(CH₂)₀₋₄C(O)R^{∘}; —C(S)R^{∘}; —(CH₂)₀₋₄C(O)OR^{∘}; —(CH₂)₀₋₄C(O)SR^{∘}; —(CH₂)₀₋₄C(O)OSiR^{∘}₃; —(CH₂)₀₋₄O C(O)R^{∘}; —OC(O)(CH₂)₀₋₄SR—, —SC(S)SR^{∘}; —(CH₂)₀₋₄SC(O)R^{∘}; —(CH₂)₀₋₄C(O)NR⁰²; —C(S)NR^{∘}₂; —C(S)SR^{∘}; —(CH₂)₀₋₄O C(O)NR⁰²; —C(O)N(OR^{∘})R^{∘}; —C(O)C(O)R^{∘}; —C(O)CH₂C(O)R^{∘}; —C(NOR^{∘})R^{∘}; —(CH₂)₀₋₄SSR^{∘}; —(CH₂)₀₋₄S(O)₂R^{∘}; —(CH₂)₀₋₄S(O)₂OR^{∘}; —(CH₂)₀₋₄O S(O)₂R^{∘}; —S(O)₂NR^{∘}₂; —(CH₂)₀₋₄S(O)R^{∘}; —N(R^{∘})S(O)₂NR^{∘}₂; —N(R^{∘})S(O)₂R^{∘}; —N(OR^{∘})R^{∘}; —C(NH)NR^{∘}₂; —P(O)₂R^{∘}; —P(O)R^{∘}₂; —OP(O)R^{∘}₂; —OP(O)(OR^{∘})₂; SiR^{∘}₃; —(C₁₋₄ straight or branched alkylene)O—N(R^{∘})₂; or —(C₁₋₄ straight or branched alkylene)C(O)O—N(R^{∘})₂, wherein each R^{∘} may be substituted as defined below and is independently hydrogen, C₁₋₆ aliphatic, C₃₋₇ cycloalkyl, a 4-7 membered heterocycloalkyl, —CH₂Ph, —O(CH₂)₀₋₁Ph, —CH₂-(5-6 membered heteroaryl ring), or a 5-6-membered saturated, partially unsaturated, or aryl ring having 0-4 heteroatoms independently selected from nitrogen, oxygen, or sulfur, or, notwithstanding the definition above, two independent occurrences of R^{∘}, taken together with their intervening atom(s), form a 3-12-membered saturated, partially unsaturated, or aryl mono- or bicyclic ring having 0-4 heteroatoms independently selected from nitrogen, oxygen, or sulfur, which may be substituted as defined below.

Suitable monovalent substituents on R⁶⁰² (or the ring formed by taking two independent occurrences of R⁶⁰² together with their intervening atoms), are independently halogen, —(CH₂)₀₋₂R^{•}, -(haloR^{•}), —(CH₂)₀₋₂OH, —(CH₂)₀₋₂OR^{•}, —(CH₂)₀₋₂CH(OR^{•})₂; —O(haloR^{•}), —CN, —N₃, —(CH₂)₀₋₂C(O)R^{•}, —(CH₂)₀₋₂C(O)OH, —(CH₂)₀₋₂C(O)OR^{•}, —(CH₂)₀₋₂SR^{•}, —(CH₂)₀₋₂SH, —(CH₂)₀₋₂NH₂, —(CH₂)₀₋₂NHR^{•}, —(CH₂)₀₋₂NR^{•}₂, —NO₂, —SiR^{•}₃, —OSiR^{•}₃, —C(O)SR^{•}, —(C₁₋₄ straight or branched alkylene)C(O)OR^{•}, or —SSR^{•} wherein each R^{•} is unsubstituted or where preceded by “halo” is substituted only with one or more halogens, and is independently selected from C₁₋₄ aliphatic, —CH₂Ph, —O(CH₂)₀₋₁Ph, or a 5-6-membered saturated, partially unsaturated, or aryl ring having 0-4 heteroatoms independently selected from nitrogen, oxygen, or sulfur.

“Acceptable” or “pharmaceutically acceptable”, with respect to a formulation, composition or ingredient, as used herein, means having no persistent detrimental effect on the general health of the subject being treated or does not abrogate the biological activity or properties of the compound, and is relatively nontoxic.

“Subject” is used to mean an animal, preferably a mammal, including a human or non-human. The terms patient and subject may be used interchangeably.

“Amelioration” of the symptoms of a particular disease, disorder or condition by administration of a particular compound or pharmaceutical composition refers to any lessening of severity, delay in onset, slowing of progression, or shortening of duration, whether permanent or temporary, lasting or transient that can be attributed to or associated with administration of the compound or composition.

“Bioavailability” refers to the percentage of the weight of the free base of a compound of Formula (I), (I-1), (I-1a), (I-1b), (I-2), (I-2a), (I-2b), (I-3), (I-4), or (I-5) that is delivered into the general circulation of the animal or human being studied following administration of compound of Formula (I), (I-1), (I-1a), (I-1b), (I-2), (I-2a), (I-2b), (I-4), or (I-5). The total exposure (AUC_{(0-∞)}) of a drug when administered intravenously is usually defined as 100% bioavailable (F %). “Oral bioavailability” refers to the extent to which the free base of compound of Formula (I), (I-1), (I-1a), (I-1b), (I-2), (I-2a), (I-2b), (I-4), or (I-5) is absorbed into the general circulation when a pharmaceutical composition comprising a compound of Formula (I), (I-1), (I-1a), (I-1b), (I-2), (I-2a), (I-2b), (I-4), or (I-5) is taken orally as compared to intravenous injection.

“Blood plasma concentration” refers to the concentration of the free base of compound of Formula (I), (I-1), (I-1a), (I-1b), (I-2), (I-2a), (I-2b), (I-4), or (I-5) in the plasma component of blood of a subject following administration of a salt of compound of Formula (I), (I-1), (I-1a), (I-1b), (I-2), (I-2a), (I-2b), (I-4), or (I-5). It is understood that the plasma concentration of the free base of compound of Formula (I), (I-1), (I-1a), (I-1b), (I-2), (I-2a), (I-2b), (I-4), or (I-5) may vary significantly between subjects, due to variability with respect to metabolism and/or possible interactions with other therapeutic agents. In accordance with some embodiments disclosed herein, the blood plasma concentration of the free base of compound of Formula (I), (I-1), (I-1a), (I-1b), (I-2), (I-2a), (I-2b), (I-4), or (I-5) may vary from subject to subject. Likewise, values such as maximum plasma concentration (Cₘₐₓ) or time to reach maximum plasma concentration (Tₘₐₓ), or total area under the plasma concentration time curve (AUC_{(0-∞)}) may vary from subject to subject. Due to this variability, the amount necessary to constitute “a therapeutically effective amount” of a compound of Formula (I), (I-1), (I-1a), (I-1b), (I-2), (I-2a), (I-2b), (I-4), or (I-5) may vary from subject to subject.

The terms “co-administration” or the like, as used herein, are meant to encompass administration of the selected therapeutic agents to a single patient, and are intended to include treatment regimens in which the agents are administered by the same or different route of administration or at the same or different time.

The terms “effective amount” or “therapeutically effective amount,” as used herein, refer to a sufficient amount of an agent or a compound being administered which will relieve to some extent one or more of the symptoms of the disease or condition being treated. The result can be reduction and/or alleviation of the signs, symptoms, or causes of a disease, or any other desired alteration of a biological system. For example, an “effective amount” for therapeutic uses is the amount of the composition including a compound as disclosed herein required to provide a clinically significant decrease in disease symptoms without undue adverse side effects. An appropriate “effective amount” in any individual case may be determined using techniques, such as a dose escalation study. The term “therapeutically effective amount” includes, for example, a prophylactically effective amount. An “effective amount” of a compound disclosed herein is an amount effective to achieve a desired pharmacologic effect or therapeutic improvement without undue adverse side effects. It is understood that “an effect amount” or “a therapeutically effective amount” can vary from subject to subject, due to variation in metabolism of Formula (I), (I-1), (I-1a), (I-1b), (I-2), (I-2a), (I-2b), (I-4), or (1-5), general condition of the subject, the condition being treated, the severity of the condition being treated, and the judgment of the prescribing physician. By way of example only, therapeutically effective amounts may be determined by routine experimentation, including but not limited to a dose escalation clinical trial.

“Enhance” or “enhancing” means to increase or prolong either in potency or duration a desired effect. By way of example, “enhancing” the effect of therapeutic agents refers to the ability to increase or prolong, either in potency or duration, the effect of therapeutic agents on during treatment of a disease, disorder or condition. An “enhancing-effective amount,” as used herein, refers to an amount adequate to enhance the effect of a therapeutic agent in the treatment of a disease, disorder or condition. When used in a patient, amounts effective for this use will depend on the severity and course of the disease, disorder or condition, previous therapy, the patient's health status and response to the drugs, and the judgment of the treating physician.

“Treat,” “treating” or “treatment”, as used herein, include alleviating, abating or ameliorating a disease or condition symptoms, preventing additional symptoms, ameliorating or preventing the underlying metabolic causes of symptoms, inhibiting the disease or condition, e.g., arresting the development of the disease or condition, relieving the disease or condition, causing regression of the disease or condition, relieving a condition caused by the disease or condition, or stopping the symptoms of the disease or condition. The terms “treat,” “treating” or “treatment”, include, but are not limited to, prophylactic and/or therapeutic treatments.

As used herein, the IC₅₀ refers to an amount, concentration or dosage of a particular test compound that achieves a 50% inhibition of a maximal response, in an assay that measures such response.

As used herein, EC₅₀ refers to a dosage, concentration or amount of a particular test compound that elicits a dose-dependent response at 50% of maximal expression of a particular response that is induced, provoked or potentiated by the particular test compound.

Methods described herein include administering to a subject in need a composition containing a therapeutically effective amount of one or more compounds described herein.

A number of animal models are useful for establishing a range of therapeutically effective doses of a compound of Formula (I), (I-1), (I-1a), (I-1b), (I-2), (I-2a), (I-2b), (I-4), or (I-5) for treating any of the foregoing diseases.

The therapeutic efficacy of a compound of Formula (I), (I-1), (I-1a), (I-1b), (I-2), (I-2a), (I-2b), (I-4), or (I-5) for one of the foregoing diseases can be optimized during a course of treatment.

### Compounds of Formula I

Described herein are compounds of Formula I:

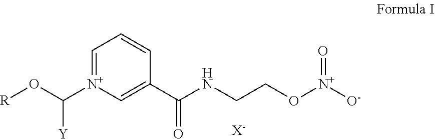

or a stereoisomer thereof;
wherein X⁻ indicates one or more counter ions;
R is selected from

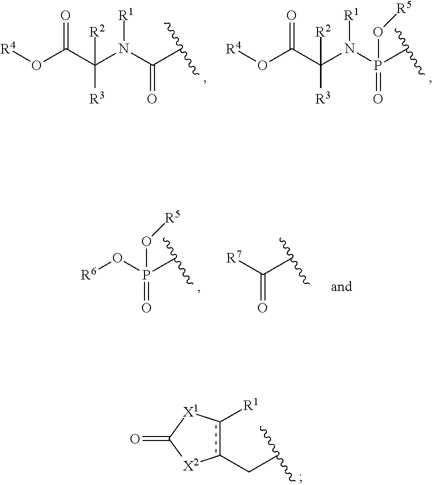

Y is H or C₁-C₄ alkyl;
R¹ is H or C₁-C₄ alkyl;
R² is H or C₁-C₄ alkyl;
R³ is H or unsubstituted or substituted C₁-C₄ alkyl; or R¹ and R³ are joined together to form a 4-7 membered substituted or unsubstituted heterocycloalkyl;
R⁴ is H or C₁-C₅ alkyl;
R⁵ and R⁶ are independently selected from hydrogen, C₁-C₅ alkyl, a 6-10 membered substituted or unsubstituted aryl, and a 6-10 membered substituted or unsubstituted heteroaryl;
R⁷ is H or C₁-C₁₂ alkyl;
X¹ and X² are independently selected from NH, O, and S; and
[symbol] is a single or double bond;
wherein when Y is H, R⁷ is not —CH(CH₃)₂, —CH₂CH(CH₃)₂, —C(CH₃)₃, or —CH₂C(CH₃)₃.

In certain embodiments, the compound of Formula (I) is a compound of Formula (I-1):

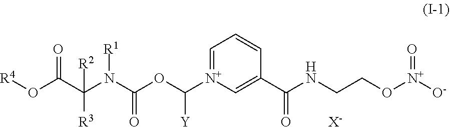

or a stereoisomer thereof.

In certain embodiments, the compound of Formula (I-1) is a compound of Formula (I-1a):

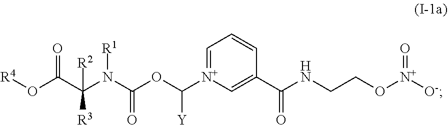

or a stereoisomer thereof.

In certain embodiments, the compound of Formula (I-1) is a compound of Formula (I-1b):

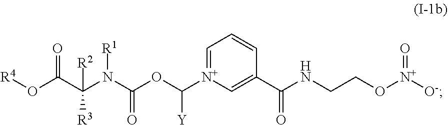

or a stereoisomer thereof.

In certain embodiments, the compound of Formula (I) is a compound of Formula (I-2):

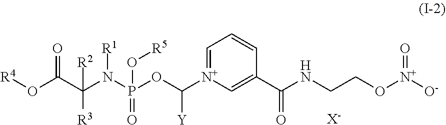

or a stereoisomer thereof.

In certain embodiments, the compound of Formula (I-1) is a compound of Formula (I-2a):

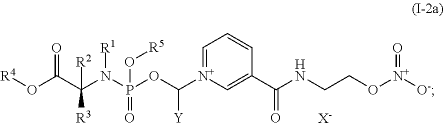

or a stereoisomer thereof.

In certain embodiments, the compound of Formula (I-1) is a compound of Formula (I-2b):

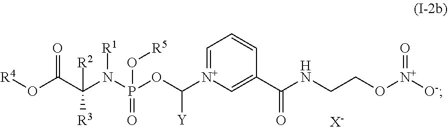

or a stereoisomer thereof.

In certain embodiments, the compound of Formula (I) is a compound of Formula (I-3):

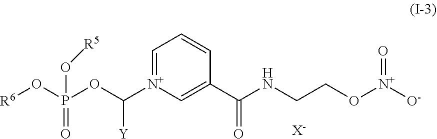

or a stereoisomer thereof.

In certain embodiments, the compound of Formula (I) is a compound of Formula (I-4):

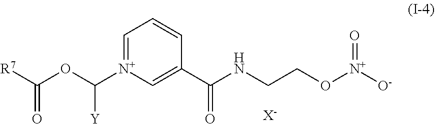

or a stereoisomer thereof.

In certain embodiments, the compound of Formula (I) is a compound of Formula (I-5):

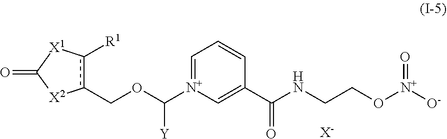

or a stereoisomer thereof.

In some embodiments of Formula (I), (I-1), (I-1a), (I-1b), (I-2), (I-2a), (I-2b), (I-3), (I-4), or (I-5), including any of the foregoing, Y is hydrogen.

In some embodiments of Formula (I), (I-1), (I-1a), (I-1b), (I-2), (I-2a), (I-2b), (I-3), (I-4), or (I-5), including any of the foregoing, Y is C₁-C₄ alkyl.

In some embodiments of Formula (I), (I-1), (I-1a), (I-1b), (I-2), (I-2a), (I-2b), (I-3), (I-4), or (I-5), including any of the foregoing, Y is Me or Et.

In some embodiments of Formula (I), (I-1), (I-1a), (I-1b), (I-2), (I-2a), (I-2b), or (I-5), including any of the foregoing, R¹ is hydrogen.

In some embodiments of Formula (I), (I-1), (I-1a), (I-1b), (I-2), (I-2a), (I-2b), or (I-5), including any of the foregoing, R¹ is C₁-C₄ alkyl.

In some embodiments of Formula (I), (I-1), (I-1a), (I-1b), (I-2), (I-2a), (I-2b), or (I-5), including any of the foregoing, R¹ is Me or Et.

In some embodiments of Formula (I), (I-1), (I-1a), (I-1b), (I-2), (I-2a), or (I-2b), including any of the foregoing, R² is C₁-C₄ alkyl.

In some embodiments of Formula (I), (I-1), (I-1a), (I-1b), (I-2), (I-2a), or (I-2b), including any of the foregoing, R² is H, Me, Et, or i-Pr.

In some embodiments of Formula (I), (I-1), (I-1a), (I-1b), (I-2), (I-2a), or (I-2b), including any of the foregoing, R² is H or Me.

In some embodiments of Formula (I), (I-1), (I-1a), (I-1b), (I-2), (I-2a), or (I-2b), including any of the foregoing, R² is H.

In some embodiments of Formula (I), (I-1), (I-1a), (I-1b), (I-2), (I-2a), or (I-2b), including any of the foregoing, R³ is H or substituted or unsubstituted C₁-C₄ alkyl.

In some embodiments of Formula (I), (I-1), (I-1a), (I-1b), (I-2), (I-2a), or (I-2b), including any of the foregoing, R³ is H.

In some embodiments of Formula (I), (I-1), (I-1a), (I-1b), (I-2), (I-2a), or (I-2b), including any of the foregoing, R³ is unsubstituted C₁-C₄ alkyl.

In some embodiments of Formula (I), (I-1), (I-1a), (I-1b), (I-2), (I-2a), or (I-2b), including any of the foregoing, R³ is —CH₃, —CH(CH₃)CH₂—CH₃, —CH₂—CH—(CH₃)₂, or —CH—(CH₃)₂.

In some embodiments of Formula (I), (I-1), (I-1a), (I-1b), (I-2), (I-2a), or (I-2b), including any of the foregoing, R³ is H, Me, Et, or i-Pr.

In some embodiments of Formula (I), (I-1), (I-1a), (I-1b), (I-2), (I-2a), or (I-2b), including any of the foregoing, R³ is Me, Et, i-Pr, n-Pr, n-Bu, i-Bu, sec-Bu, or t-Bu.

In some embodiments of Formula (I), (I-1), (I-1a), (I-1b), (I-2), (I-2a), or (I-2b), including any of the foregoing, R³ is Me or i-Bu.

In some embodiments of Formula (I), (I-1), (I-1a), (I-1b), (I-2), (I-2a), or (I-2b), including any of the foregoing, R³ is H or Me.

In some embodiments of Formula (I), (I-1), (I-1a), (I-1b), (I-2), (I-2a), or (I-2b), including any of the foregoing, R³ is Me.

In some embodiments of Formula (I), (I-1), (I-1a), (I-1b), (I-2), (I-2a), or (I-2b), including any of the foregoing, R³ is substituted C₁-C₄ alkyl and the substitutions are selected from guanidine, hydroxyl, amide, carboxylic acid, heteroalkyl, heteroaryl, amine, and aryl.

In some embodiments of Formula (I), (I-1), (I-1a), (I-1b), (I-2), (I-2a), or (I-2b), including any of the foregoing, R³ is substituted C₁-C₄ alkyl and the substitutions are selected from hydroxyl, heteroalkyl, heteroaryl, aryl, —COOH, —CONH₂, and —NH₂.

In some embodiments of Formula (I), (I-1), (I-1a), (I-1b), (I-2), (I-2a), or (I-2b), including any of the foregoing, R³ is substituted C₁-C₄ alkyl and the substitutions are selected from —NH—C(NH₂)═NH, —CONH₂, —COOH, —SH, -1H-imidazole, —NH₂, —S—CH₃, -Ph, —OH, -1H-indole, and -Ph-OH.

In some embodiments of Formula (I), (I-1), (I-1a), (I-1b), (I-2), (I-2a), or (I-2b), including any of the foregoing, R³ is substituted C₁-C₄ alkyl and the substitutions are selected from —CONH₂, -Ph, and —OH.

In some embodiments of Formula (I), (I-1), (I-1a), (I-1b), (I-2), (I-2a), or (I-2b), including any of the foregoing, R³ is selected from H, —CH₃, —(CH₂)₃—NH—C(NH₂)═NH, —CH₂—CONH₂, —CH₂—COOH, —CH₂—SH, —(CH₂)₂—CONH₂, —(CH₂)₂—COOH, —CH₂-1H-imidazole, —CH(CH₃)CH₂—CH₃, —CH₂—CH—(CH₃)₂, —(CH₂)₄—NH₂, —(CH₂)₂—S—CH₃, —CH₂-Ph, —CH₂—OH, —CH(OH)—CH₃, —CH₂-1H-indole, —CH₂-Ph-OH, and —CH—(CH₃)₂.

In some embodiments of Formula (I), (I-1), (I-1a), (I-1b), (I-2), (I-2a), or (I-2b), including any of the foregoing, R³ is selected from —CH₃, —CH₂—OH, —CH₂-Ph, —CH₂—CONH₂, and CH₂—C(H)—(CH₃)₂.

In some embodiments of Formula (I), (I-1), (I-1a), (I-1b), (I-2), (I-2a), or (I-2b), including any of the foregoing, R¹ and R³ are joined together to form a 4-7 membered substituted or unsubstituted heterocycloalkyl.

In some embodiments of Formula (I), (I-1), (I-1a), (I-1b), (I-2), (I-2a), or (I-2b), including any of the foregoing, R¹ and R³ are joined together to form a 4-7 membered unsubstituted heterocycloalkyl.

In some embodiments of Formula (I), (I-1), (I-1a), (I-1b), (I-2), (I-2a), or (I-2b), including any of the foregoing, R¹ and R³ are joined together to form unsubstituted pyrrolidinyl, piperidinyl, or morpholinyl.

In some embodiments of Formula (I), (I-1), (I-1a), (I-1b), (I-2), (I-2a), or (I-2b), including any of the foregoing, R¹ and R³ are joined together to form a unsubstituted pyrrolidinyl.

In some embodiments of Formula (I), (I-1), (I-1a), (I-1b), (I-2), (I-2a), or (I-2b), including any of the foregoing, R⁴ is H.

In some embodiments of Formula (I), (I-1), (I-1a), (I-1b), (I-2), (I-2a), or (I-2b), including any of the foregoing, R⁴ is C₁-C₅ alkyl.

In some embodiments of Formula (I), (I-1), (I-1a), (I-1b), (I-2), (I-2a), or (I-2b), including any of the foregoing, R⁴ is Me, Et, i-Pr, n-Pr, n-Bu, i-Bu, sec-Bu, or t-Bu.

In some embodiments of Formula (I), (I-1), (I-1a), (I-1b), (I-2), (I-2a), or (I-2b), including any of the foregoing, R⁴ is H, Me, Et, or i-Pr.

In some embodiments of Formula (I), (I-1), (I-1a), (I-1b), (I-2), (I-2a), or (I-2b), including any of the foregoing, R⁴ is Me, Et, or i-Pr.

In some embodiments of Formula (I), (I-1), (I-1a), (I-1b), (I-2), (I-2a), or (I-2b), including any of the foregoing, R⁴ is Me or Et.

In some embodiments of Formula (I), (I-1), (I-1a), (I-1b), (I-2), (I-2a), or (I-2b), including any of the foregoing, R⁴ is i-Pr.

In some embodiments of Formula (I), (I-1), (I-1a), (I-1b), (I-2), (I-2a), (I-2b) or (I-3), including any of the foregoing, R⁵ is H.

In some embodiments of Formula (I), (I-1), (I-1a), (I-1b), (I-2), (I-2a), (I-2b) or (I-3), including any of the foregoing, R⁵ is C₁-C₅ alkyl.

In some embodiments of Formula (I), (I-1), (I-1a), (I-1b), (I-2), (I-2a), (I-2b) or (I-3), including any of the foregoing, R⁵ is Me, Et, i-Pr, n-Pr, n-Bu, i-Bu, sec-Bu, or t-Bu.

In some embodiments of Formula (I), (I-1), (I-1a), (I-1b), (I-2), (I-2a), (I-2b) or (I-3), including any of the foregoing, R⁵ is Me or Et.

In some embodiments of Formula (I), (I-1), (I-1a), (I-1b), (I-2), (I-2a), (I-2b) or (I-3), including any of the foregoing, R⁵ is a 6-10 membered substituted or unsubstituted aryl.

In some embodiments of Formula (I), (I-1), (I-1a), (I-1b), (I-2), (I-2a), (I-2b) or (I-3), including any of the foregoing, R⁵ is a 6-10 membered unsubstituted aryl.

In some embodiments of Formula (I), (I-1), (I-1a), (I-1b), (I-2), (I-2a), (I-2b) or (I-3), including any of the foregoing, R⁵ is unsubstituted phenyl or naphthyl.

In some embodiments of Formula (I), (I-1), (I-1a), (I-1b), (I-2), (I-2a), (I-2b) or (I-3), including any of the foregoing, R⁵ is a 6-10 membered substituted or unsubstituted heteroaryl.

In some embodiments of Formula (I-3), including any of the foregoing, R⁵ and R⁶ are independently selected from H and C₁-C₅ alkyl.

In some embodiments of Formula (I-3), including any of the foregoing, R⁵ and R⁶ are independently selected from H and C₁-C₅ alkyl wherein C₁-C₅ alkyl is Me, Et, i-Pr, n-Pr, n-Bu, i-Bu, sec-Bu, or t-Bu.

In some embodiments of Formula (I-3), including any of the foregoing, R⁵ and R⁶ are independently selected from H and C₁-C₅ alkyl wherein C₁-C₅ alkyl is Me, Et, or i-Pr.

In some embodiments of Formula (I-3), including any of the foregoing, R⁵ is H and R⁶ is C₁-C₅ alkyl.

In some embodiments of Formula (I-3), including any of the foregoing, R⁵ is H and R⁶ is C₁-C₅ alkyl wherein C₁-C₅ alkyl is Me, Et, i-Pr, n-Pr, n-Bu, i-Bu, sec-Bu, or t-Bu.

In some embodiments of Formula (I-3), including any of the foregoing, R⁵ is H and R⁶ is C₁-C₅ alkyl wherein C₁-C₅ alkyl is Me, Et, or i-Pr.

In some embodiments of Formula (I-3), including any of the foregoing, R⁵ and R⁶ are both H.

In some embodiments of Formula (I-3), including any of the foregoing, R⁵ and R⁶ are both C₁-C₅ alkyl.

In some embodiments of Formula (I-3), including any of the foregoing, R⁵ and R⁶ are both methyl.

In some embodiments of Formula (I-3), including any of the foregoing, R⁵ and R⁶ are both ethyl.

In some embodiments of Formula (I-3), including any of the foregoing, R⁵ and R⁶ are independently selected from H and a 6-10 membered substituted or unsubstituted aryl.

In some embodiments of Formula (I-3), including any of the foregoing, R⁵ is H and R⁶ is phenyl.

In some embodiments of Formula (I-4), including any of the foregoing, R⁷ is H.

In some embodiments of Formula (I-4), including any of the foregoing, R⁷ is C₁-C₁₂ alkyl.

In some embodiments of Formula (I-4), including any of the foregoing, R⁷ is C₁-C₂ alkyl.

In some embodiments of Formula (I-4), including any of the foregoing, R⁷ is C₆-C₁₂ alkyl.

In some embodiments of Formula (I-4), including any of the foregoing, R⁷ is C₁₀-C₁₂ alkyl.

In some embodiments of Formula (I-4), including any of the foregoing, R⁷ is straight C₁-C₁₂ alkyl.

In some embodiments of Formula (I-4), including any of the foregoing, R⁷ is branched C₁-C₁₂ alkyl.

In some embodiments of Formula (I-4), including any of the foregoing, R⁷ is branched C₁-C₆ alkyl.

In some embodiments of Formula (I-4), including any of the foregoing, R⁷ is straight C₆-C₁₂ alkyl.

In some embodiments of Formula (I-4), including any of the foregoing, R⁷ is Me or Et.

In some embodiments of Formula (I-4), including any of the foregoing, R⁷ is Me.

In some embodiments of Formula (I-4), including any of the foregoing, R⁷ is Et.

In some embodiments of Formula (I-4), including any of the foregoing, R⁷ is —C(CH₃)₂CH₂CH₃.

In some embodiments of Formula (I-4), including any of the foregoing, R⁷ is —(CH₂)₁₁.

In some embodiments of Formula (I-4), including any of the foregoing, Y is H and R⁷ is C₁-C₁₂ alkyl.

In some embodiments of Formula (I-5), including any of the foregoing, X¹ is O.

In some embodiments of Formula (I-5), including any of the foregoing, X¹ is NH.

In some embodiments of Formula (I-5), including any of the foregoing, X¹ is S.

In some embodiments of Formula (I-5), including any of the foregoing, X² is O.

In some embodiments of Formula (I-5), including any of the foregoing, X² is NH.

In some embodiments of Formula (I-5), including any of the foregoing, X² is S.

In some embodiments of Formula (I-5), including any of the foregoing, X¹ is O and X² is O.

In some embodiments of Formula (I-5), including any of the foregoing, X¹ is O and X² is S.

In some embodiments of Formula (I-5), including any of the foregoing, X¹ is O and X² is NH.

In some embodiments of Formula (I-5), including any of the foregoing, R¹ is H.

In some embodiments of Formula (I-5), including any of the foregoing, R¹ is C₁-C₄ alkyl.

In some embodiments of Formula (I-5), including any of the foregoing, R¹ is Me or Et.

In some embodiments of Formula (I-5), including any of the foregoing, [symbol] is a single bond.

In some embodiments of Formula (I-5), including any of the foregoing, [symbol] is a double bond.

In some embodiments of Formula (I-5), including any of the foregoing,

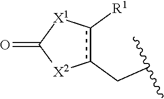

is selected from

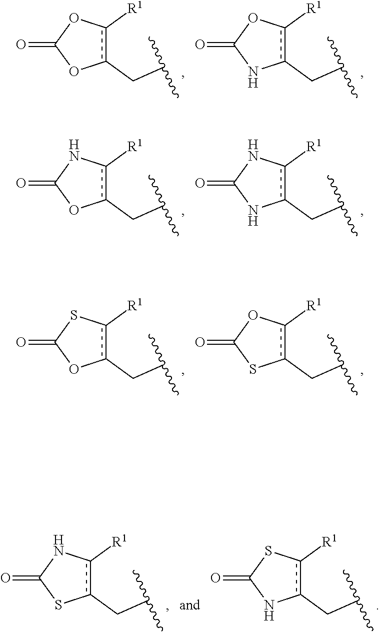

In some embodiments of Formula (I-5), including any of the foregoing,

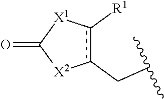

is selected from

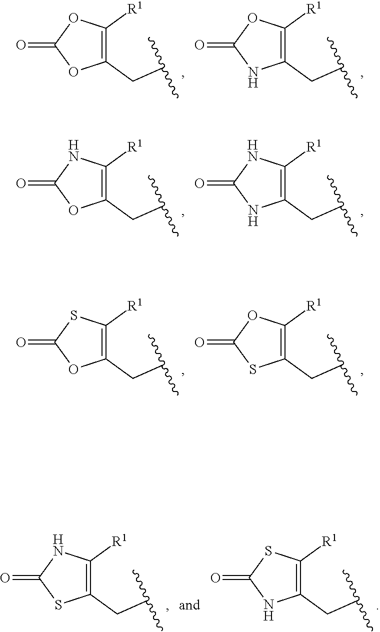

In some embodiments of Formula (I-5), including any of the foregoing,

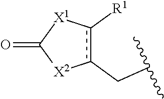

is selected from

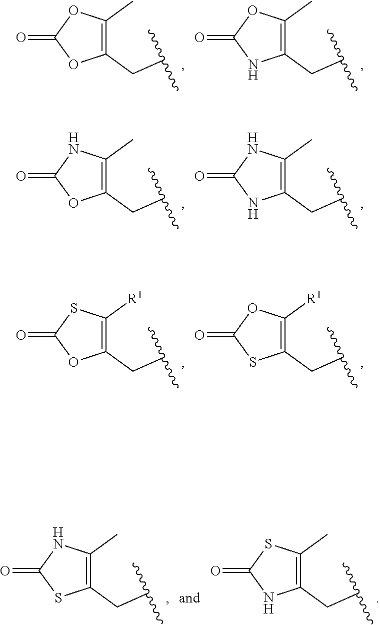

In some embodiments of Formula (I-5), including any of the foregoing,

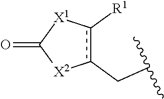

is

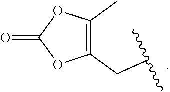 [SMILES: *Cc1oc(=O)oc1C]

In some embodiments of Formula (I-1), (I-1a), or (I-1b), including any of the foregoing, Y is hydrogen, R¹ is hydrogen, and R² is hydrogen.

In some embodiments of Formula (I-1), (I-1a), or (I-1b), including any of the foregoing, Y is hydrogen, R¹ is methyl, and R² is hydrogen.

In some embodiments of Formula (I-1), (I-1a), or (I-1b), including any of the foregoing, Y is hydrogen, R¹ is hydrogen, and R² is methyl.

In some embodiments of Formula (I-1), (I-1a), or (I-1b), including any of the foregoing, Y is hydrogen, R¹ is hydrogen, R² is hydrogen, and R⁴ is C₁-C₈ alkyl.

In some embodiments of Formula (I-1), (I-1a), or (I-1b), including any of the foregoing, Y is hydrogen, R¹ is hydrogen, R² is hydrogen, and R⁴ is Me, Et, or iPr.

In some embodiments of Formula (I-1), (I-1a), or (I-1b), including any of the foregoing, Y is hydrogen, R¹ is hydrogen, R² is hydrogen, R³ is substituted or unsubstituted C₁-C₄ alkyl, and R⁴ is C₁-C₈ alkyl.

In some embodiments of Formula (I-1), (I-1a), or (I-1b), including any of the foregoing, Y is hydrogen, R¹ is hydrogen, R² is hydrogen, R³ is substituted or unsubstituted C₁-C₄ alkyl, and R⁴ is Me, Et, or iPr.

In some embodiments of Formula (I-2), (I-2a), or (I-2b), including any of the foregoing, Y is hydrogen, R¹ is hydrogen, and R² is hydrogen.

In some embodiments of Formula (I-2), (I-2a), or (I-2b), including any of the foregoing, Y is hydrogen, R¹ is methyl, and R² is hydrogen.

In some embodiments of Formula (I-2), (I-2a), or (I-2b), including any of the foregoing, Y is hydrogen, R¹ is hydrogen, and R² is methyl.

In some embodiments of Formula (I-2), (I-2a), or (I-2b), including any of the foregoing, Y is hydrogen, R¹ is hydrogen, R² is hydrogen, and R⁴ is C₁-C₅ alkyl.

In some embodiments of Formula (I-2), (I-2a), or (I-2b), including any of the foregoing, Y is hydrogen, R¹ is hydrogen, R² is hydrogen, and R⁴ is Me, Et, or iPr.

In some embodiments of Formula (I-2), (I-2a), or (I-2b), including any of the foregoing, Y is hydrogen, R¹ is hydrogen, R² is hydrogen, R⁴ is C₁-C₅ alkyl, and R⁵ is a 6-10 membered unsubstituted aryl.

In some embodiments of Formula (I-2), (I-2a), or (I-2b), including any of the foregoing, Y is hydrogen, R¹ is hydrogen, R² is hydrogen, R⁴ is Me, Et, or iPr, and R⁵ is a 6-10 membered unsubstituted aryl.

In some embodiments of Formula (I-2), (I-2a), or (I-2b), including any of the foregoing, Y is hydrogen, R¹ is hydrogen, R² is hydrogen, R⁴ is Me, Et, or iPr, and R⁵ is unsubstituted phenyl.

In some embodiments of Formula (I-2), (I-2a), or (I-2b), including any of the foregoing, Y is hydrogen, R¹ is hydrogen, R² is hydrogen, R³ is substituted or unsubstituted C₁-C₄ alkyl,
R⁴ is C₁-C₅ alkyl, and R⁵ is a 6-10 membered unsubstituted aryl.

In some embodiments of Formula (I-2), (I-2a), or (I-2b), including any of the foregoing, Y is hydrogen, R¹ is hydrogen, R² is hydrogen, R³ is substituted or unsubstituted C₁-C₄ alkyl,
R⁴ is Me, Et, or iPr, and R⁵ is a 6-10 membered unsubstituted aryl.

In some embodiments of Formula (I-2), (I-2a), or (I-2b), including any of the foregoing, Y is hydrogen, R¹ is hydrogen, R² is hydrogen, R³ is unsubstituted C₁-C₄ alkyl, R⁴ is Me, Et, or iPr, and R⁵ is a 6-10 membered unsubstituted aryl.

In some embodiments of Formula (I-3), including any of the foregoing, Y is H and R⁵ and R⁶ are independently selected from hydrogen, C₁-C₅ alkyl, and a 6-10 membered substituted or unsubstituted aryl.

In some embodiments of Formula (I-3), including any of the foregoing, Y is H and R⁵ and R⁶ are independently selected from hydrogen and C₁-C₅ alkyl.

In some embodiments of Formula (I-4), including any of the foregoing, Y is H and R⁷ is C₁-C₂ alkyl.

In some embodiments of Formula (I-5), including any of the foregoing, Y is H, R¹ is C₁-C₄ alkyl, X¹ is O, and X² is O.

In some embodiments of Formula (I-5), including any of the foregoing, Y is H, R¹ is C₁-C₄ alkyl, X¹ is NH, and X² is O.

In some embodiments of Formula (I-5), including any of the foregoing, Y is H, R¹ is C₁-C₄ alkyl, X¹ is O, and X² is NH.

In some embodiments of Formula (I), (I-1), (I-1a), (I-1b), (I-2), (I-2a), (I-2b), (I-3), (I-4), or (I-5), including any of the foregoing, X⁻ is selected from F⁻, Cl⁻, Br⁻, I⁻, MeSO₂O—, acetate, carbonate, chromate, citrate, fumarate, lactate, malonate, nitrate, phosphate, tartrate, trifluoroacetate, succinate, formate and sulfate.

In some embodiments of Formula (I), (I-1), (I-1a), (I-1b), (I-2), (I-2a), (I-2b), (I-3), (I-4), or (I-5), including any of the foregoing, X⁻ is selected from nitrate and formate.

In some embodiments of Formula (I), (I-1), (I-1a), (I-1b), (I-2), (I-2a), (I-2b), (I-3), (I-4), or (I-5), including any of the foregoing, X⁻ is nitrate.

In some embodiments of Formula (I), (I-1), (I-1a), (I-1b), (I-2), (I-2a), (I-2b), (I-3), (I-4), or (I-5), including any of the foregoing, X⁻ is formate.

In some embodiments, Formula (I-1) is selected from:

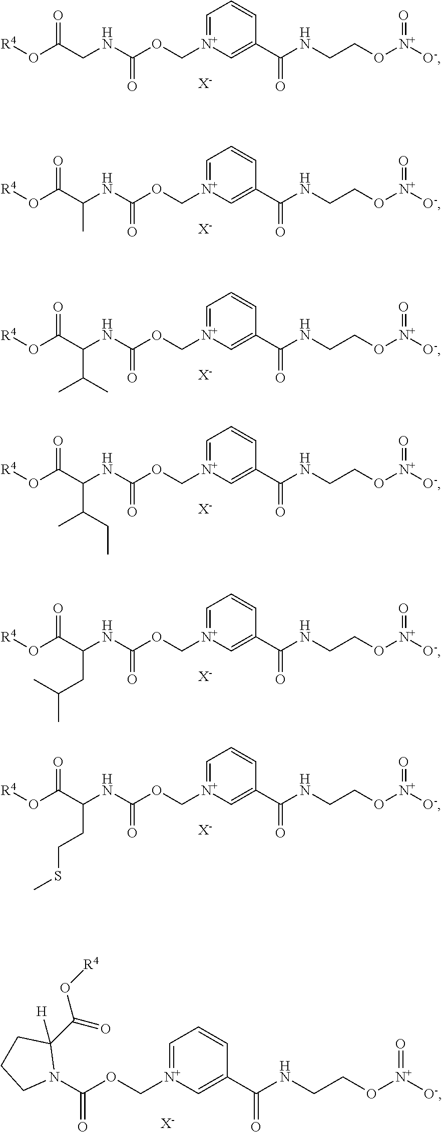

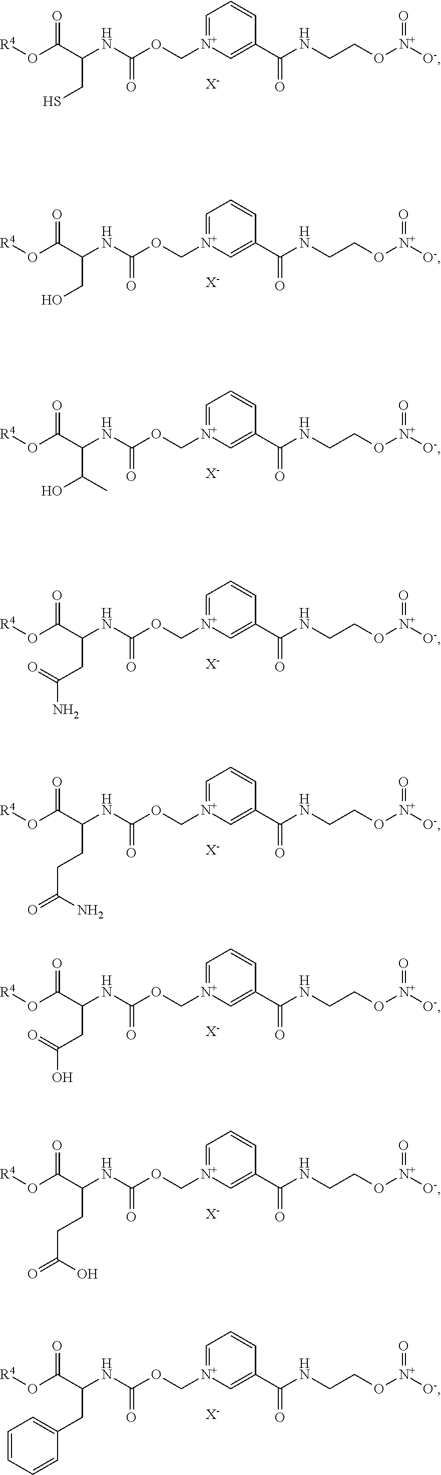

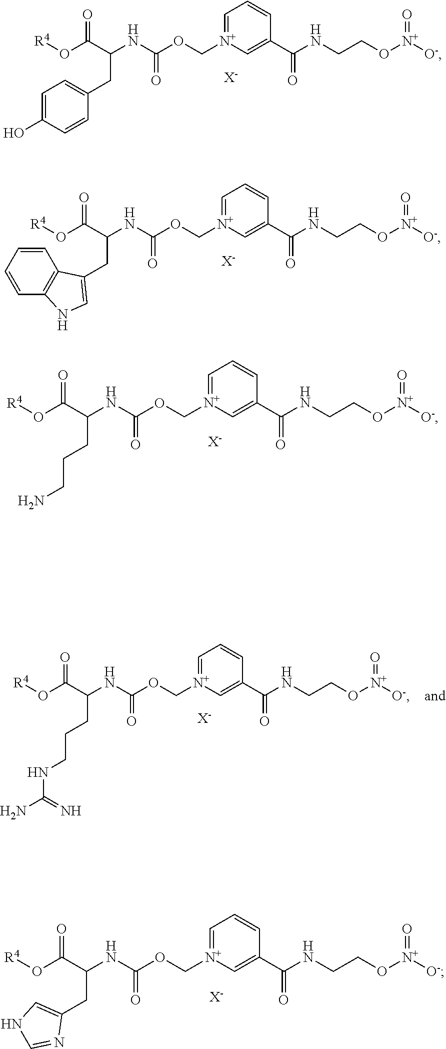

or a stereoisomer thereof.

In some embodiments, Formula (I-1a) is selected from:

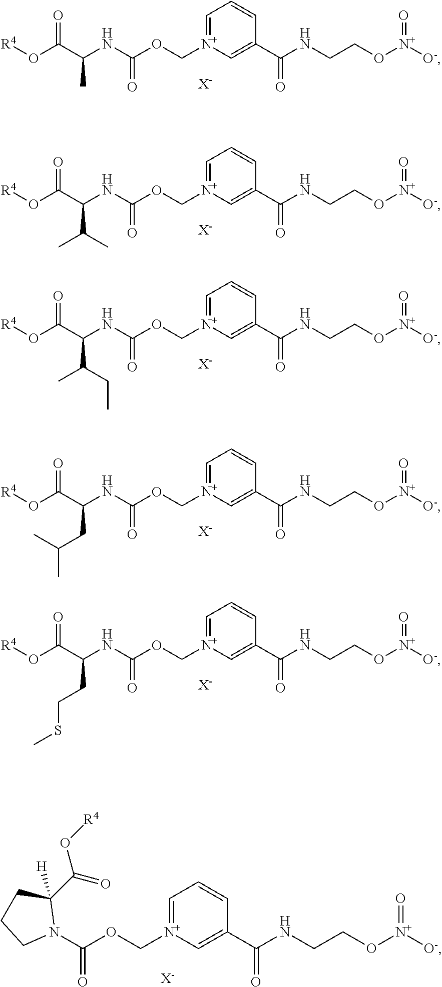

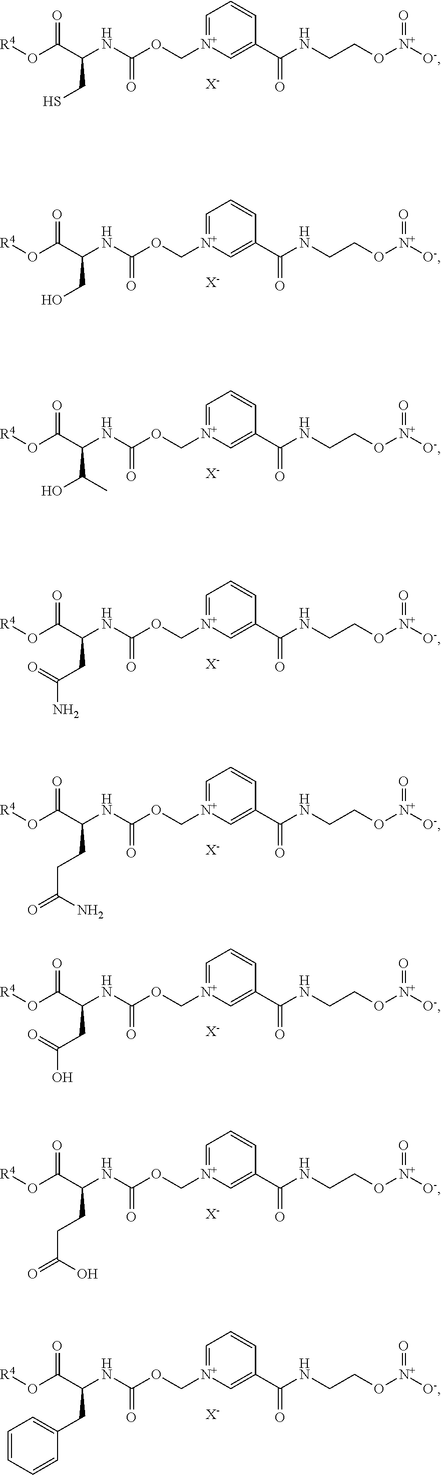

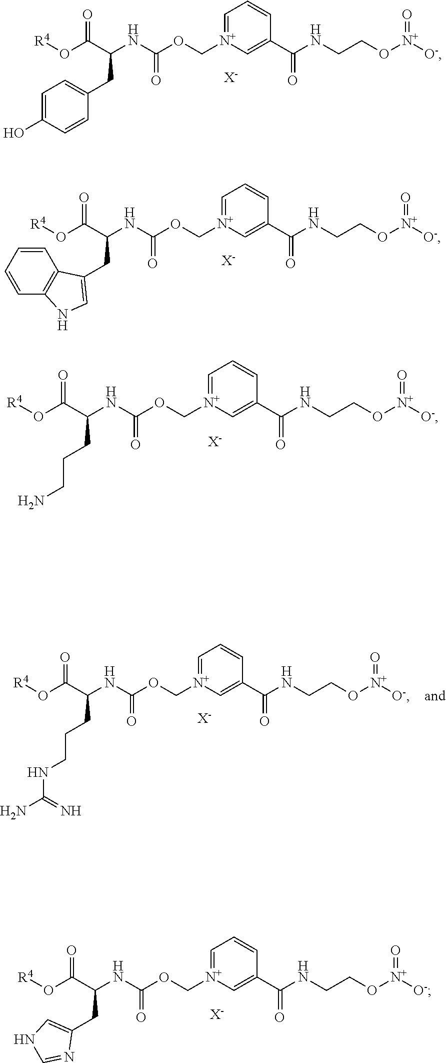

or a stereoisomer thereof.

In some embodiments, Formula (I-1b) is selected from:

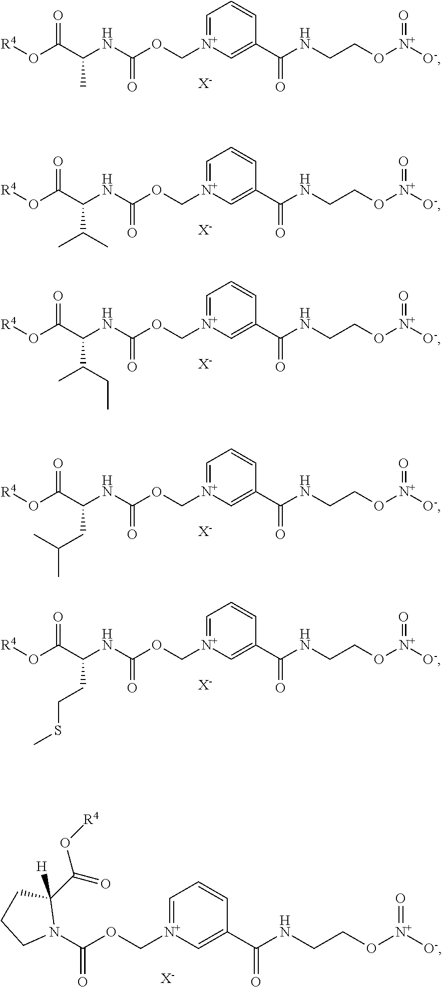

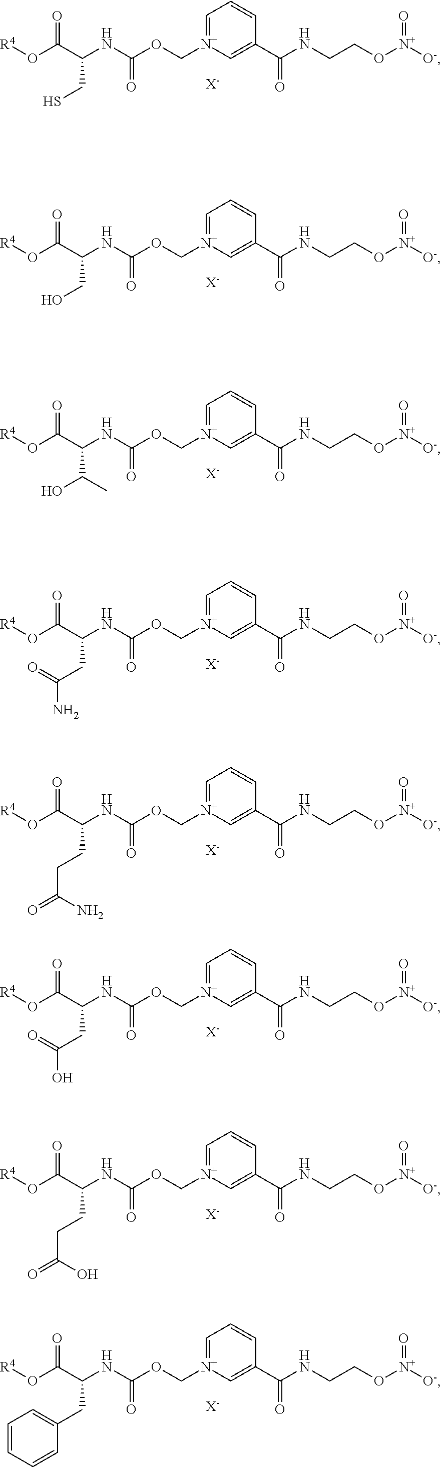

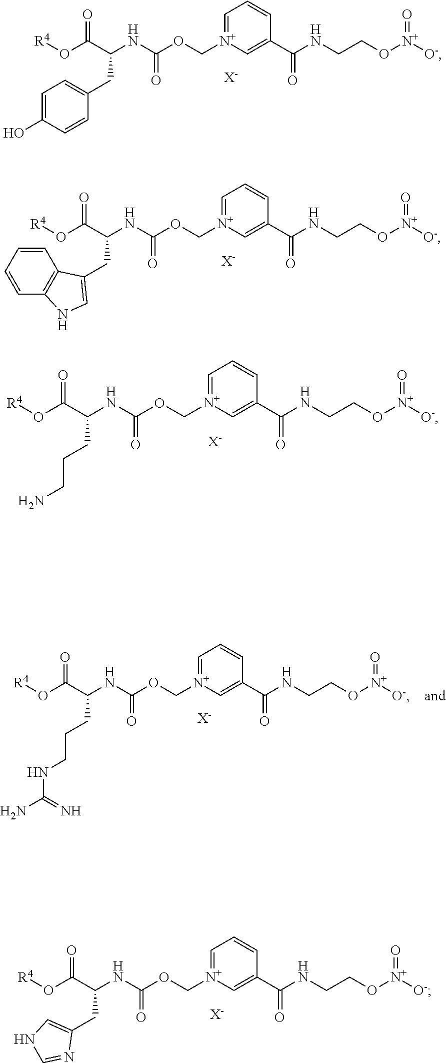

or a stereoisomer thereof.

In some embodiments, Formula (I) is selected from:

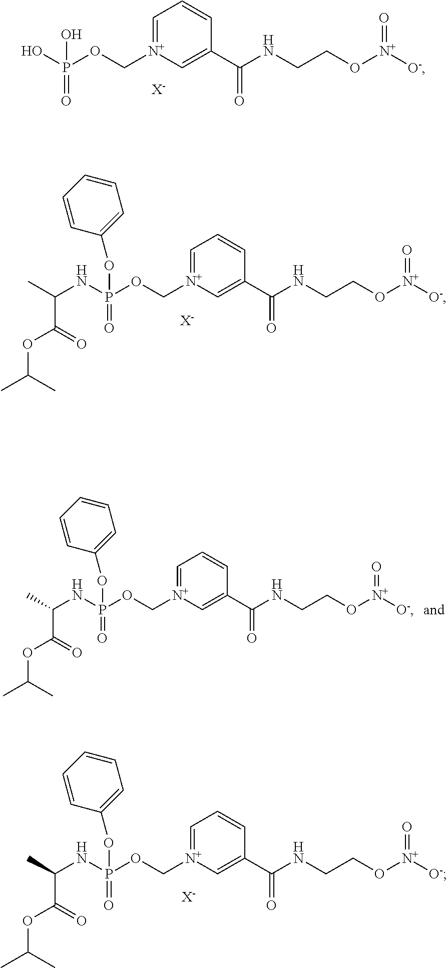

or a stereoisomer thereof.

In some embodiments, Formula (I-1) is selected from:

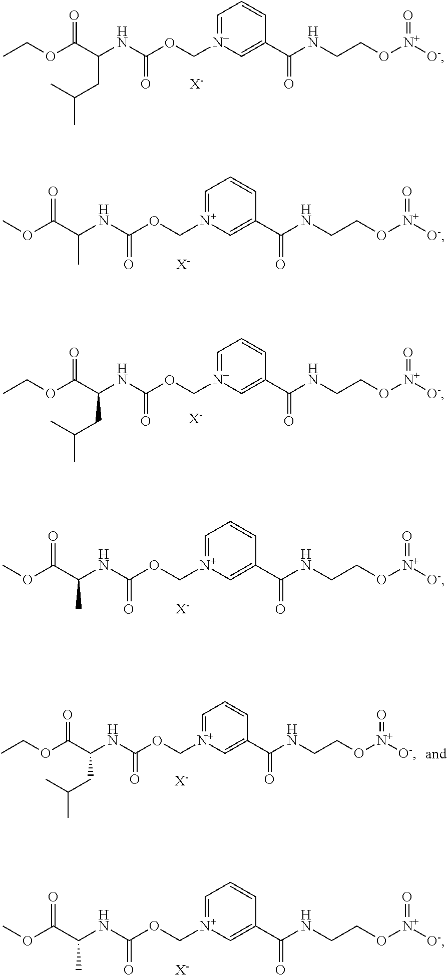

In some embodiments, Formula (I-1) is selected from:

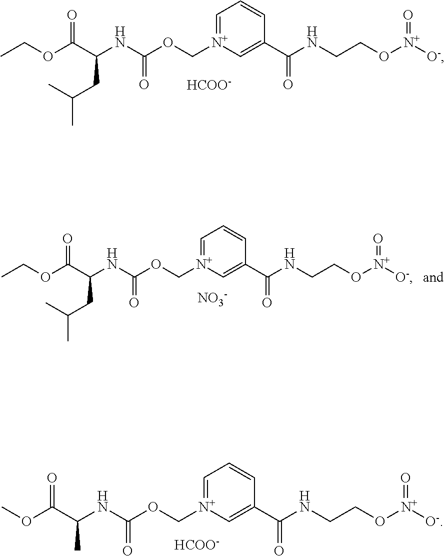

In one embodiment, Formula (I-4) is

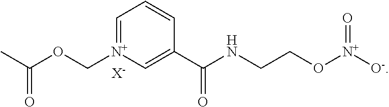

In one embodiment, Formula (I-4) is selected from:

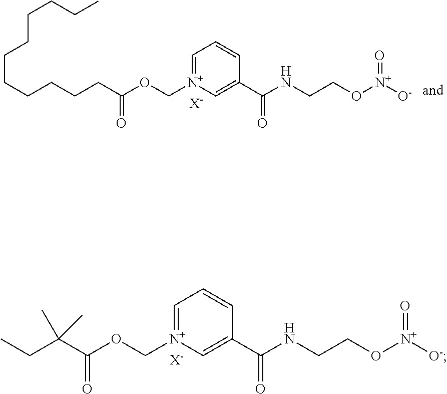

In one embodiment, Formula (I-5) is

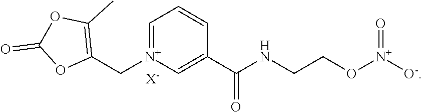

In one embodiment, Formula (1-5) is

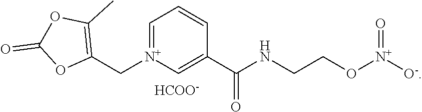

Compounds disclosed herein have a structure of Formula (I). It is understood that when reference is made to compounds described herein, it is meant to include compounds of any of Formula (I), (I-1) (I-1a), (I-1b), (I-2), (I-2a), (I-2b), (I-3), (I-4), or (I-5) as well as to all of the specific compounds that fall within the scope of these generic formulae, unless otherwise indicated.

Compounds described herein may possess one or more stereocenters and each center may exist in the R or S configuration. Compounds presented herein include all diastereomeric, enantiomeric, and epimeric forms as well as the appropriate mixtures thereof. Stereoisomers may be obtained, if desired, by methods known in the art as, for example, the separation of stereoisomers by chiral chromatographic columns.

Diastereomeric mixtures can be separated into their individual diastereomers on the basis of their physical chemical differences by methods known, for example, by chromatography and/or fractional crystallization. In some embodiments, enantiomers can be separated by chiral chromatographic columns. In some embodiments, enantiomers can be separated by converting the enantiomeric mixture into a diastereomeric mixture by reaction with an appropriate optically active compound (e.g., alcohol), separating the diastereomers and converting (e.g., hydrolyzing) the individual diastereomers to the corresponding pure enantiomers. All such isomers, including diastereomers, enantiomers, and mixtures thereof are considered as part of the compositions described herein.

Methods and formulations described herein include the use of N-oxides, crystalline forms, or pharmaceutically acceptable salts of compounds described herein, as well as active metabolites of these compounds having the same type of activity. In some situations, compounds may exist as tautomers. All tautomers are included within the scope of the compounds presented herein. In addition, compounds described herein can exist in unsolvated as well as solvated forms with pharmaceutically acceptable solvents such as water, ethanol, and the like. Solvated forms of compounds presented herein are also considered to be disclosed herein.

Compounds described herein include isotopically-labeled compounds, which are identical to those recited in the various formulas and structures presented herein, but for the fact that one or more atoms are replaced by an atom having an atomic mass or mass number different from the atomic mass or mass number usually found in nature. Examples of isotopes that can be incorporated into the present compounds include isotopes of hydrogen, carbon, nitrogen, oxygen, fluorine and chlorine, such as ²H, ³H, ¹³C, ¹⁴C, ¹⁵N, ¹⁸O, ¹⁷O, ³⁵S, ¹⁸F, ³⁶Cl, respectively. Certain isotopically-labeled compounds described herein, for example those into which radioactive isotopes such as ³H and ¹⁴C are incorporated, are useful in drug and/or substrate tissue distribution assays. Further, substitution with isotopes such as deuterium, i.e., ²H, can afford certain therapeutic advantages resulting from greater metabolic stability, for example increased in vivo half-life or reduced dosage requirements.

The salts are recovered by using at least one of the following techniques: filtration, precipitation with a non-solvent followed by filtration, evaporation of the solvent, or, in the case of aqueous solutions, lyophilization.

Throughout the specification, groups and substituents thereof can be chosen by one skilled in the field to provide stable moieties and compounds.

In one embodiment, a compound of Formula (I-1a), (I-1b), (I-2a), or (I-2b) is at least about 50% free, at least about 60% free, at least about 70% free, at least about 75% free, at least about 80% free, at least about 85% free, at least about 90% free, at least about 95% free, at least about 97% free, at least about 98% free, at least about 99% free, or even 100% free by weight of the opposite (R) or (S)-enantiomer based solely on the weight of compound of Formula (I-1a), (I-1b), (I-2a), or (I-2b) (i.e., excluding the weight of the salt). In one embodiment, the compound of Formula (I-1a), (I-1b), (I-2a), or (I-2b) is about 85% to about 95% free of the opposite (R) or (S)-enantiomer based solely on the weight of compound of Formula (I-1a), (I-1b), (I-2a), or (I-2b). In one embodiment, the compound of Formula (I-1a), (I-1b), (I-2a), or (I-2b) is about 95% to about 100% free of the opposite (R)- or (S)-enantiomer based solely on the weight of compound of Formula (I-1a), (1-1b), (I-2a), or (I-2b).

### Pharmaceutical Composition/Formulation

Pharmaceutical compositions may be formulated in a conventional manner using one or more physiologically acceptable carriers including excipients and auxiliaries which facilitate processing of the active compounds into preparations which can be used pharmaceutically. Proper formulation is dependent upon the route of administration chosen. Any of the well-known techniques, carriers, and excipients may be used as suitable and as understood in the art. A summary of pharmaceutical compositions described herein may be found, for example, in Remington: The Science and Practice of Pharmacy, Nineteenth Ed (Easton, Pa.: Mack Publishing Company, 1995); Hoover, John E., Remington's Pharmaceutical Sciences, Mack Publishing Co., Easton, Pennsylvania 1975; Liberman, H. A. and Lachman, L., Eds., Pharmaceutical Dosage Forms, Marcel Decker, New York, N.Y., 1980; and Pharmaceutical Dosage Forms and Drug Delivery Systems, Seventh Ed. (Lippincott Williams & Wilkins 1999), herein incorporated by reference in their entirety.

A pharmaceutical composition, as used herein, refers to a mixture of a compound described herein, such as, for example, compounds of any of Formula (I), (I-1), (I-1a), (I-1b), (I-2), (I-2a), (I-2b), (I-3), (I-4), or (I-5) with other chemical components, such as carriers, stabilizers, diluents, dispersing agents, suspending agents, thickening agents, and/or excipients. The pharmaceutical composition facilitates administration of the compound to an organism. In practicing the methods of treatment or use provided herein, therapeutically effective amounts of compounds described herein are administered in a pharmaceutical composition to a mammal having a disease, disorder, or condition to be treated. Preferably, the mammal is a human. A therapeutically effective amount can vary widely depending on the severity of the disease, the age and relative health of the subject, the potency of the compound used and other factors. The compounds can be used singly or in combination with one or more therapeutic agents as components of mixtures.

In certain embodiments, compositions may also include one or more pH adjusting agents or buffering agents, including acids such as acetic, boric, citric, lactic, phosphoric and hydrochloric acids; bases such as sodium hydroxide, sodium phosphate, sodium borate, sodium citrate, sodium acetate, sodium lactate and tris-hydroxymethylaminomethane; and buffers such as citrate/dextrose, sodium bicarbonate and ammonium chloride. Such acids, bases and buffers are included in an amount required to maintain pH of the composition in an acceptable range.

In some embodiments, compositions may also include one or more salts in an amount required to bring osmolality of the composition into an acceptable range. Such salts include those having sodium, potassium or ammonium cations and chloride, citrate, ascorbate, borate, phosphate, bicarbonate, sulfate, thiosulfate or bisulfite anions; suitable salts include sodium chloride, potassium chloride, sodium thiosulfate, sodium bisulfite and ammonium sulfate.

The term “pharmaceutical combination” as used herein, means a product that results from the mixing or combining of more than one active ingredient and includes both fixed and non-fixed combinations of the active ingredients. The term “fixed combination” means that the active ingredients, e.g. a compound described herein and a co-agent, are both administered to a patient simultaneously in the form of a single entity or dosage. The term “non-fixed combination” means that the active ingredients, e.g. a compound described herein and a co-agent, are administered to a patient as separate entities either simultaneously, concurrently or sequentially with no specific intervening time limits, wherein such administration provides effective levels of the two compounds in the body of the patient. The latter also applies to cocktail therapy, e.g. the administration of three or more active ingredients.

The pharmaceutical compositions described herein can be administered to a subject by multiple administration routes, including but not limited to, oral, parenteral (e.g., intravenous, subcutaneous, intramuscular), intranasal, buccal, topical, rectal, or transdermal administration routes. The pharmaceutical compositions described herein include, but are not limited to, aqueous liquid dispersions, self-emulsifying dispersions, solid solutions, liposomal dispersions, aerosols, solid dosage forms, powders, immediate release formulations, controlled release formulations, fast melt formulations, tablets, capsules, pills, delayed release formulations, extended release formulations, pulsatile release formulations, multiparticulate formulations, and mixed immediate and controlled release formulations.

Pharmaceutical compositions including a compound described herein may be manufactured in a conventional manner, such as, by way of example only, by means of conventional mixing, dissolving, granulating, dragee-making, levigating, emulsifying, encapsulating, entrapping or compression processes.

The pharmaceutical compositions will include at least one compound described herein, such as, for example, a compound of any of Formula (I), (I-1), (I-1a), (I-1b), (I-2), (I-2a), (I-2b), (I-3), (I-4), or (I-5) as an active ingredient in free-acid or free-base form, or in a pharmaceutically acceptable salt form. In addition, the methods and pharmaceutical compositions described herein include the use of N-oxides, crystalline forms, as well as active metabolites of these compounds having the same type of activity. In some situations, compounds may exist as tautomers. All tautomers are included within the scope of the compounds presented herein. Additionally, the compounds described herein can exist in unsolvated as well as solvated forms with pharmaceutically acceptable solvents such as water, ethanol, and the like. The solvated forms of the compounds presented herein are also considered to be disclosed herein.

“Antifoaming agents” reduce foaming during processing which can result in coagulation of aqueous dispersions, bubbles in the finished film, or generally impair processing. Exemplary anti-foaming agents include silicon emulsions or sorbitan sesquoleate.

“Antioxidants” include, for example, butylated hydroxytoluene (BHT), sodium ascorbate, ascorbic acid, sodium metabisulfite and tocopherol. In certain embodiments, antioxidants enhance chemical stability where required.

In certain embodiments, compositions provided herein may also include one or more preservatives to inhibit microbial activity. Suitable preservatives include mercury-containing substances such as merfen and thiomersal; stabilized chlorine dioxide; and quaternary ammonium compounds such as benzalkonium chloride, cetyltrimethylammonium bromide and cetylpyridinium chloride.

Formulations described herein may benefit from antioxidants, metal chelating agents, thiol containing compounds and other general stabilizing agents. Examples of such stabilizing agents, include, but are not limited to: (a) about 0.5% to about 2% w/v glycerol, (b) about 0.1% to about 1% w/v methionine, (c) about 0.1% to about 2% w/v monothioglycerol, (d) about 1 mM to about 10 mM EDTA, (e) about 0.01% to about 2% w/v ascorbic acid, (f) 0.003% to about 0.02% w/v polysorbate 80, (g) 0.001% to about 0.05% w/v. polysorbate 20, (h) arginine, (i) heparin, (j) dextran sulfate, (k) cyclodextrins, (1) pentosan polysulfate and other heparinoids, (m) divalent cations such as magnesium and zinc; or (n) combinations thereof.

“Binders” impart cohesive qualities and include, e.g., alginic acid and salts thereof; cellulose derivatives such as carboxymethylcellulose, methylcellulose (e.g., Methocel®), hydroxypropylmethylcellulose, hydroxyethylcellulose, hydroxypropylcellulose (e.g., Klucel®), ethylcellulose (e.g., Ethocel®), and microcrystalline cellulose (e.g., Avicel®); microcrystalline dextrose; amylose; magnesium aluminum silicate; polysaccharide acids; bentonites; gelatin; polyvinylpyrrolidone/vinyl acetate copolymer; crosspovidone; povidone; starch; pregelatinized starch; tragacanth, dextrin, a sugar, such as sucrose (e.g., Dipac®), glucose, dextrose, molasses, mannitol, sorbitol, xylitol (e.g., Xylitab®), and lactose; a natural or synthetic gum such as acacia, tragacanth, ghatti gum, mucilage of isapol husks, polyvinylpyrrolidone (e.g., Polyvidone® CL, Kollidon® CL, Polyplasdone® XL-10), larch arabogalactan, Veegum®, polyethylene glycol, waxes, sodium alginate, and the like.

A “carrier” or “carrier materials” include any commonly used excipients in pharmaceutics and should be selected on the basis of compatibility with compounds disclosed herein, such as, compounds of any of Formula (I), (I-1), (I-1a), (I-1b), (I-2), (I-2a), (I-2b), (I-3), (I-4), or (I-5) and the release profile properties of the desired dosage form. Exemplary carrier materials include, e.g., binders, suspending agents, disintegration agents, filling agents, surfactants, solubilizers, stabilizers, lubricants, wetting agents, diluents, and the like. “Pharmaceutically compatible carrier materials” may include, but are not limited to, acacia, gelatin, colloidal silicon dioxide, calcium glycerophosphate, calcium lactate, maltodextrin, glycerine, magnesium silicate, polyvinylpyrrollidone (PVP), cholesterol, cholesterol esters, sodium caseinate, soy lecithin, taurocholic acid, phosphotidylcholine, sodium chloride, tricalcium phosphate, dipotassium phosphate, cellulose and cellulose conjugates, sugars sodium stearoyl lactylate, carrageenan, monoglyceride, diglyceride, pregelatinized starch, and the like. See, e.g., Remington: The Science and Practice of Pharmacy, Nineteenth Ed (Easton, Pa.: Mack Publishing Company, 1995); Hoover, John E., Remington's Pharmaceutical Sciences, Mack Publishing Co., Easton, Pennsylvania 1975; Liberman, H. A. and Lachman, L., Eds., Pharmaceutical Dosage Forms, Marcel Decker, New York, N.Y., 1980; and Pharmaceutical Dosage Forms and Drug Delivery Systems, Seventh Ed. (Lippincott Williams & Wilkins 1999).

“Dispersing agents,” and/or “viscosity modulating agents” include materials that control the diffusion and homogeneity of a drug through liquid media or a granulation method or blend method. In some embodiments, these agents also facilitate the effectiveness of a coating or eroding matrix. Exemplary diffusion facilitators/dispersing agents include, e.g., hydrophilic polymers, electrolytes, Tween® 60 or 80, PEG, polyvinylpyrrolidone (PVP; commercially known as Plasdone®), and the carbohydrate-based dispersing agents such as, for example, hydroxypropyl celluloses (e.g., HPC, HPC-SL, and HPC-L), hydroxypropyl methylcelluloses (e.g., HPMC K100, HPMC K4M, HPMC K15M, and HPMC K100M), carboxymethylcellulose sodium, methylcellulose, hydroxyethylcellulose, hydroxypropylcellulose, hydroxypropylmethylcellulose phthalate, hydroxypropylmethylcellulose acetate stearate (HPMCAS), noncrystalline cellulose, magnesium aluminum silicate, triethanolamine, polyvinyl alcohol (PVA), vinyl pyrrolidone/vinyl acetate copolymer (S630), 4-(1,1,3,3-tetramethylbutyl)-phenol polymer with ethylene oxide and formaldehyde (also known as tyloxapol), poloxamers (e.g., Pluronics F68®, F88®, and F108®, which are block copolymers of ethylene oxide and propylene oxide); and poloxamines (e.g., Tetronic 908®, also known as Poloxamine 908®, which is a tetrafunctional block copolymer derived from sequential addition of propylene oxide and ethylene oxide to ethylenediamine (BASF Corporation, Parsippany, N.J.)), polyvinylpyrrolidone K12, polyvinylpyrrolidone K17, polyvinylpyrrolidone K25, or polyvinylpyrrolidone K30, polyvinylpyrrolidone/vinyl acetate copolymer (S-630), polyethylene glycol, e.g., the polyethylene glycol can have a molecular weight of about 300 to about 6000, or about 3350 to about 4000, or about 7000 to about 5400, sodium carboxymethylcellulose, methylcellulose, polysorbate-80, sodium alginate, gums, such as, e.g., gum tragacanth and gum acacia, guar gum, xanthans, including xanthan gum, sugars, cellulosics, such as, e.g., sodium carboxymethylcellulose, methylcellulose, sodium carboxymethylcellulose, polysorbate-80, sodium alginate, polyethoxylated sorbitan monolaurate, polyethoxylated sorbitan monolaurate, povidone, carbomers, polyvinyl alcohol (PVA), alginates, chitosans and combinations thereof. Plasticizers such as cellulose or triethyl cellulose can also be used as dispersing agents. Dispersing agents particularly useful in liposomal dispersions and self-emulsifying dispersions are dimyristoyl phosphatidyl choline, natural phosphatidyl choline from eggs, natural phosphatidyl glycerol from eggs, cholesterol and isopropyl myristate.

Combinations of one or more erosion facilitator with one or more diffusion facilitator can also be used in the present compositions.

The term “diluent” refers to chemical compounds that are used to dilute the compound of interest prior to delivery. Diluents can also be used to stabilize compounds because they can provide a more stable environment. Salts dissolved in buffered solutions (which also can provide pH control or maintenance) are utilized as diluents in the art, including, but not limited to a phosphate buffered saline solution. In certain embodiments, diluents increase bulk of the composition to facilitate compression or create sufficient bulk for homogenous blend for capsule filling. Such compounds include e.g., lactose, starch, mannitol, sorbitol, dextrose, microcrystalline cellulose such as Avicel®; dibasic calcium phosphate, dicalcium phosphate dihydrate; tricalcium phosphate, calcium phosphate; anhydrous lactose, spray-dried lactose; pregelatinized starch, compressible sugar, such as Di-Pac® (Amstar); mannitol, hydroxypropylmethylcellulose, hydroxypropylmethylcellulose acetate stearate, sucrose-based diluents, confectioner's sugar; monobasic calcium sulfate monohydrate, calcium sulfate dihydrate; calcium lactate trihydrate, dextrates; hydrolyzed cereal solids, amylose; powdered cellulose, calcium carbonate; glycine, kaolin; mannitol, sodium chloride; inositol, bentonite, and the like.

The term “disintegrate” includes both the dissolution and dispersion of the dosage form when contacted with gastrointestinal fluid. “Disintegration agents or disintegrants” facilitate the breakup or disintegration of a substance. Examples of disintegration agents include a starch, e.g., a natural starch such as corn starch or potato starch, a pregelatinized starch such as National 1551 or Amijel®, or sodium starch glycolate such as Promogel® or Explotab®, a cellulose such as a wood product, methylcrystalline cellulose, e.g., Avicel®, Avicel® PH101, Avicel® PH102, Avicel® PH105, Elcema® P100, Emcocel®, Vivacel®, Ming Tia®, and Solka-Floc®, methylcellulose, croscarmellose, or a cross-linked cellulose, such as cross-linked sodium carboxymethylcellulose (Ac-Di-Sol®), cross-linked carboxymethylcellulose, or cross-linked croscarmellose, a cross-linked starch such as sodium starch glycolate, a cross-linked polymer such as crosspovidone, a cross-linked polyvinylpyrrolidone, alginate such as alginic acid or a salt of alginic acid such as sodium alginate, a clay such as Veegum® HV (magnesium aluminum silicate), a gum such as agar, guar, locust bean, Karaya, pectin, or tragacanth, sodium starch glycolate, bentonite, a natural sponge, a surfactant, a resin such as a cation-exchange resin, citrus pulp, sodium lauryl sulfate, sodium lauryl sulfate in combination starch, and the like.

“Drug absorption” or “absorption” typically refers to the process of movement of drug from site of administration of a drug across a barrier into a blood vessel or the site of action, e.g., a drug moving from the gastrointestinal tract into the portal vein or lymphatic system.

An “enteric coating” is a substance that remains substantially intact in the stomach but dissolves and releases the drug in the small intestine or colon. Generally, the enteric coating comprises a polymeric material that prevents release in the low pH environment of the stomach but that ionizes at a higher pH, typically a pH of 6 to 7, and thus dissolves sufficiently in the small intestine or colon to release the active agent therein.

“Erosion facilitators” include materials that control the erosion of a particular material in gastrointestinal fluid. Erosion facilitators are generally known to those of ordinary skill in the art. Exemplary erosion facilitators include, e.g., hydrophilic polymers, electrolytes, proteins, peptides, and amino acids.

“Filling agents” include compounds such as lactose, calcium carbonate, calcium phosphate, dibasic calcium phosphate, calcium sulfate, microcrystalline cellulose, cellulose powder, dextrose, dextrates, dextran, starches, pregelatinized starch, sucrose, xylitol, lactitol, mannitol, sorbitol, sodium chloride, polyethylene glycol, and the like.

“Flavoring agents” and/or “sweeteners” useful in the formulations described herein, include, e.g., acacia syrup, acesulfame K, alitame, anise, apple, aspartame, banana, Bavarian cream, berry, black currant, butterscotch, calcium citrate, camphor, caramel, cherry, cherry cream, chocolate, cinnamon, bubble gum, citrus, citrus punch, citrus cream, cotton candy, cocoa, cola, cool cherry, cool citrus, cyclamate, cylamate, dextrose, eucalyptus, eugenol, fructose, fruit punch, ginger, glycyrrhetinate, glycyrrhiza (licorice) syrup, grape, grapefruit, honey, isomalt, lemon, lime, lemon cream, monoammonium glyrrhizinate (MagnaSweet®), maltol, mannitol, maple, marshmallow, menthol, mint cream, mixed berry, neohesperidine DC, neotame, orange, pear, peach, peppermint, peppermint cream, Prosweet® Powder, raspberry, root beer, rum, saccharin, safrole, sorbitol, spearmint, spearmint cream, strawberry, strawberry cream, stevia, sucralose, sucrose, sodium saccharin, saccharin, aspartame, acesulfame potassium, mannitol, talin, sylitol, sucralose, sorbitol, Swiss cream, tagatose, tangerine, thaumatin, tutti fruitti, vanilla, walnut, watermelon, wild cherry, wintergreen, xylitol, or any combination of these flavoring ingredients, e.g., anise-menthol, cherry-anise, cinnamon-orange, cherry-cinnamon, chocolate-mint, honey-lemon, lemon-lime, lemon-mint, menthol-eucalyptus, orange-cream, vanilla-mint, and mixtures thereof.

“Lubricants” and “glidants” are compounds that prevent, reduce or inhibit adhesion or friction of materials. Exemplary lubricants include, e.g., stearic acid, calcium hydroxide, talc, sodium stearyl fumerate, a hydrocarbon such as mineral oil, or hydrogenated vegetable oil such as hydrogenated soybean oil (Sterotex®), higher fatty acids and their alkali-metal and alkaline earth metal salts, such as aluminum, calcium, magnesium, zinc, stearic acid, sodium stearates, glycerol, talc, waxes, Stearowet®, boric acid, sodium benzoate, sodium acetate, sodium chloride, leucine, a polyethylene glycol (e.g., PEG-4000) or a methoxypolyethylene glycol such as Carbowax™, sodium oleate, sodium benzoate, glyceryl behenate, polyethylene glycol, magnesium or sodium lauryl sulfate, colloidal silica such as Syloid™, Cab-O-Sil®, a starch such as corn starch, silicone oil, a surfactant, and the like.

A “measurable serum concentration” or “measurable plasma concentration” describes the blood serum or blood plasma concentration, typically measured in mg, g, or ng of therapeutic agent per ml, dl, or 1 of blood serum, absorbed into the bloodstream after administration. As used herein, measurable plasma concentrations are typically measured in ng/ml or g/ml.

“Pharmacodynamics” refers to the factors which determine the biologic response observed relative to the concentration of drug at a site of action.

“Pharmacokinetics” refers to the factors which determine the attainment and maintenance of the appropriate concentration of drug at a site of action.

“Plasticizers” are compounds used to soften the microencapsulation material or film coatings to make them less brittle. Suitable plasticizers include, e.g., polyethylene glycols such as PEG 300, PEG 400, PEG 600, PEG 1450, PEG 3350, and PEG 800, stearic acid, propylene glycol, oleic acid, triethyl cellulose and triacetin. In some embodiments, plasticizers can also function as dispersing agents or wetting agents.

“Solubilizers” include compounds such as triacetin, triethylcitrate, ethyl oleate, ethyl caprylate, sodium lauryl sulfate, sodium doccusate, vitamin E TPGS, dimethylacetamide, N-methylpyrrolidone, N-hydroxyethylpyrrolidone, polyvinylpyrrolidone, hydroxypropylmethyl cellulose, hydroxypropyl cyclodextrins, ethanol, n-butanol, isopropyl alcohol, cholesterol, bile salts, polyethylene glycol 200-600, glycofurol, transcutol, propylene glycol, and dimethyl isosorbide and the like.

“Stabilizers” include compounds such as any antioxidation agents, buffers, acids, preservatives and the like.

“Steady state,” as used herein, is when the amount of drug administered is equal to the amount of drug eliminated within one dosing interval resulting in a plateau or constant plasma drug exposure.

“Suspending agents” include compounds such as polyvinylpyrrolidone, e.g., polyvinylpyrrolidone K12, polyvinylpyrrolidone K17, polyvinylpyrrolidone K25, or polyvinylpyrrolidone K30, vinyl pyrrolidone/vinyl acetate copolymer (S630), polyethylene glycol, e.g., the polyethylene glycol can have a molecular weight of about 300 to about 6000, or about 3350 to about 4000, or about 7000 to about 5400, sodium carboxymethylcellulose, methylcellulose, hydroxypropylmethylcellulose, hydroxymethylcellulose acetate stearate, polysorbate-80, hydroxyethylcellulose, sodium alginate, gums, such as, e.g., gum tragacanth and gum acacia, guar gum, xanthans, including xanthan gum, sugars, cellulosics, such as, e.g., sodium carboxymethylcellulose, methylcellulose, sodium carboxymethylcellulose, hydroxypropylmethylcellulose, hydroxyethylcellulose, polysorbate-80, sodium alginate, polyethoxylated sorbitan monolaurate, polyethoxylated sorbitan monolaurate, povidone and the like.

“Surfactants” include compounds such as sodium lauryl sulfate, sodium docusate, Tween 60 or 80, triacetin, vitamin E TPGS, sorbitan monooleate, polyoxyethylene sorbitan monooleate, polysorbates, polaxomers, bile salts, glyceryl monostearate, copolymers of ethylene oxide and propylene oxide, e.g., Pluronic® (BASF), and the like. Some other surfactants include polyoxyethylene fatty acid glycerides and vegetable oils, e.g., polyoxyethylene (60) hydrogenated castor oil; and polyoxyethylene alkylethers and alkylphenyl ethers, e.g., octoxynol 10, octoxynol 40. In some embodiments, surfactants may be included to enhance physical stability or for other purposes.

“Viscosity enhancing agents” include, e.g., methyl cellulose, xanthan gum, carboxymethyl cellulose, hydroxypropyl cellulose, hydroxypropylmethyl cellulose, hydroxypropylmethyl cellulose acetate stearate, hydroxypropylmethyl cellulose phthalate, carbomer, polyvinyl alcohol, alginates, acacia, chitosans and combinations thereof.

“Wetting agents” include compounds such as oleic acid, glyceryl monostearate, sorbitan monooleate, sorbitan monolaurate, triethanolamine oleate, polyoxyethylene sorbitan monooleate, polyoxyethylene sorbitan monolaurate, sodium docusate, sodium oleate, sodium lauryl sulfate, sodium doccusate, triacetin, Tween 80, vitamin E TPGS, ammonium salts and the like.

### Dosage Forms

The compositions described herein can be formulated for administration to a subject via any conventional means including, but not limited to, oral, parenteral (e.g., intravenous, subcutaneous, or intramuscular), buccal, intranasal, rectal or transdermal administration routes. As used herein, the term “subject” is used to mean an animal, preferably a mammal, including a human or non-human. The terms patient and subject may be used interchangeably.

Moreover, the pharmaceutical compositions described herein, which include a compound of any of Formula (I), (I-1), (I-1a), (I-1b), (I-2), (I-2a), (I-2b), (I-3), (I-4), or (I-5) can be formulated into any suitable dosage form, including but not limited to, aqueous oral dispersions, liquids, gels, syrups, elixirs, slurries, suspensions and the like, for oral ingestion by a patient to be treated, solid oral dosage forms, aerosols, controlled release formulations, fast melt formulations, effervescent formulations, lyophilized formulations, tablets, powders, pills, dragees, capsules, delayed release formulations, extended release formulations, pulsatile release formulations, multiparticulate formulations, and mixed immediate release and controlled release formulations.

Pharmaceutical preparations for oral use can be obtained by mixing one or more solid excipient with one or more of the compounds described herein, optionally grinding the resulting mixture, and processing the mixture of granules, after adding suitable auxiliaries, if desired, to obtain tablets or dragee cores. Suitable excipients include, for example, fillers such as sugars, including lactose, sucrose, mannitol, or sorbitol; cellulose preparations such as, for example, maize starch, wheat starch, rice starch, potato starch, gelatin, gum tragacanth, methylcellulose, microcrystalline cellulose, hydroxypropylmethylcellulose, sodium carboxymethylcellulose; or others such as: polyvinylpyrrolidone (PVP or povidone) or calcium phosphate. If desired, disintegrating agents may be added, such as the cross-linked croscarmellose sodium, polyvinylpyrrolidone, agar, or alginic acid or a salt thereof such as sodium alginate.

Dragee cores are provided with suitable coatings. For this purpose, concentrated sugar solutions may be used, which may optionally contain gum arabic, talc, polyvinylpyrrolidone, carbopol gel, polyethylene glycol, and/or titanium dioxide, lacquer solutions, and suitable organic solvents or solvent mixtures. Dyestuffs or pigments may be added to the tablets or dragee coatings for identification or to characterize different combinations of active compound doses.

Pharmaceutical preparations which can be used orally include push-fit capsules made of gelatin, as well as soft, sealed capsules made of gelatin and a plasticizer, such as glycerol or sorbitol. The push-fit capsules can contain the active ingredients in admixture with filler such as lactose, binders such as starches, and/or lubricants such as talc or magnesium stearate and, optionally, stabilizers. In soft capsules, the active compounds may be dissolved or suspended in suitable liquids, such as fatty oils, liquid paraffin, or liquid polyethylene glycols. In addition, stabilizers may be added. All formulations for oral administration should be in dosages suitable for such administration.

In some embodiments, the solid dosage forms disclosed herein may be in the form of a tablet, (including a suspension tablet, a fast-melt tablet, a bite-disintegration tablet, a rapid-disintegration tablet, an effervescent tablet, or a caplet), a pill, a powder (including a sterile packaged powder, a dispensable powder, or an effervescent powder) a capsule (including both soft or hard capsules, e.g., capsules made from animal-derived gelatin or plant-derived HPMC, or “sprinkle capsules”), solid dispersion, solid solution, bioerodible dosage form, controlled release formulations, pulsatile release dosage forms, multiparticulate dosage forms, pellets, granules, or an aerosol. In some embodiments, the pharmaceutical composition is in the form of a powder. In some embodiments, the pharmaceutical composition is in the form of a tablet, including but not limited to, a fast-melt tablet. Additionally, pharmaceutical compositions described herein may be administered as a single capsule or in multiple capsule dosage form. In some embodiments, the pharmaceutical composition is administered in two, or three, or four, capsules or tablets.

In some embodiments, solid dosage forms, e.g., tablets, effervescent tablets, and capsules, are prepared by mixing particles of a compound of any of Formula (I), (I-1), (I-1a), (I-1b), (I-2), (I-2a), (I-2b), (I-4), or (I-5) with one or more pharmaceutical excipients to form a bulk blend composition. When referring to these bulk blend compositions as homogeneous, it is meant that the particles of the compound of any of Formula (I), (I-1), (I-1a), (I-1b), (I-2), (I-2a), (I-2b), (I-3), (I-4), or (I-5) are dispersed evenly throughout the composition so that the composition may be readily subdivided into equally effective unit dosage forms, such as tablets, pills, and capsules. The individual unit dosages may also include film coatings, which disintegrate upon oral ingestion or upon contact with diluent. These formulations can be manufactured by conventional pharmacological techniques.

Conventional pharmacological techniques include, e.g., one or a combination of methods: (1) dry mixing, (2) direct compression, (3) milling, (4) dry or non-aqueous granulation, (5) wet granulation, or (6) fusion. See, e.g., Lachman et al., The Theory and Practice of Industrial Pharmacy (1986). Other methods include, e.g., spray drying, pan coating, melt granulation, granulation, fluidized bed spray drying or coating (e.g., wurster coating), tangential coating, top spraying, tableting, extruding and the like.

The pharmaceutical solid dosage forms described herein can include a compound described herein and one or more pharmaceutically acceptable additives such as a compatible carrier, binder, filling agent, suspending agent, flavoring agent, sweetening agent, disintegrating agent, dispersing agent, surfactant, lubricant, colorant, diluent, solubilizer, moistening agent, plasticizer, stabilizer, penetration enhancer, wetting agent, anti-foaming agent, antioxidant, preservative, or one or more combination thereof. In some embodiments, using standard coating procedures, such as those described in Remington's Pharmaceutical Sciences, 20th Edition (2000), a film coating is provided around the formulation of the compound of any of Formula (I), (I-1), (I-1a), (I-1b), (I-2), (I-2a), (I-2b), (I-3), (I-4), or (I-5). In some embodiments, some or all of the particles of the compound of any of Formula (I), (I-1), (I-1a), (I-1b), (I-2), (I-2a), (I-2b), (I-4), or (I-5) are coated. In some embodiments, some or all of the particles of the compound of any of Formula (I), (I-1), (I-1a), (I-1b), (I-2), (I-2a), (I-2b), (I-3), (I-4), or (I-5), are microencapsulated. In still some embodiments, the particles of the compound of any of Formula (I), (I-1), (I-1a), (I-1b), (I-2), (I-2a), (I-2b), (I-3), (I-4), or (I-5) are not microencapsulated and are uncoated.

Suitable carriers for use in the solid dosage forms described herein include, but are not limited to, acacia, gelatin, colloidal silicon dioxide, calcium glycerophosphate, calcium lactate, maltodextrin, glycerine, magnesium silicate, sodium caseinate, soy lecithin, sodium chloride, tricalcium phosphate, dipotassium phosphate, sodium stearoyl lactylate, carrageenan, monoglyceride, diglyceride, pregelatinized starch, hydroxypropylmethylcellulose, hydroxypropylmethylcellulose acetate stearate, sucrose, microcrystalline cellulose, lactose, mannitol and the like.

Suitable filling agents for use in the solid dosage forms described herein include, but are not limited to, lactose, calcium carbonate, calcium phosphate, dibasic calcium phosphate, calcium sulfate, microcrystalline cellulose, cellulose powder, dextrose, dextrates, dextran, starches, pregelatinized starch, hydroxypropylmethycellulose (HPMC), hydroxypropylmethycellulose phthalate, hydroxypropylmethylcellulose acetate stearate (HPMCAS), sucrose, xylitol, lactitol, mannitol, sorbitol, sodium chloride, polyethylene glycol, and the like.

In order to release the compound of any of Formula (I), (I-1), (I-1a), (I-1b), (I-2), (I-2a), (I-2b), (I-3), (I-4), or (I-5) from a solid dosage form matrix as efficiently as possible, disintegrants are often used in the formulation, especially when the dosage forms are compressed with binder. Disintegrants help rupturing the dosage form matrix by swelling or capillary action when moisture is absorbed into the dosage form. Suitable disintegrants for use in the solid dosage forms described herein include, but are not limited to, natural starch such as corn starch or potato starch, a pregelatinized starch such as National 1551 or Amijel®, or sodium starch glycolate such as Promogel® or Explotab®, a cellulose such as a wood product, methylcrystalline cellulose, e.g., Avicel®, Avicel® PH101, Avicel® PH102, Avicel® PH105, Elcema® P100, Emcocel®, Vivacel®, Ming Tia®, and Solka-Floc®, methylcellulose, croscarmellose, or a cross-linked cellulose, such as cross-linked sodium carboxymethylcellulose (Ac-Di-Sol®), cross-linked carboxymethylcellulose, or cross-linked croscarmellose, a cross-linked starch such as sodium starch glycolate, a cross-linked polymer such as crospovidone, a cross-linked polyvinylpyrrolidone, alginate such as alginic acid or a salt of alginic acid such as sodium alginate, a clay such as Veegum® HV (magnesium aluminum silicate), a gum such as agar, guar, locust bean, Karaya, pectin, or tragacanth, sodium starch glycolate, bentonite, a natural sponge, a surfactant, a resin such as a cation-exchange resin, citrus pulp, sodium lauryl sulfate, sodium lauryl sulfate in combination starch, and the like.

Binders impart cohesiveness to solid oral dosage form formulations: for powder filled capsule formulation, they aid in plug formation that can be filled into soft or hard shell capsules and for tablet formulation, they ensure the tablet remaining intact after compression and help assure blend uniformity prior to a compression or fill step. Materials suitable for use as binders in the solid dosage forms described herein include, but are not limited to, carboxymethylcellulose, methylcellulose (e.g., Methocel®), hydroxypropylmethylcellulose (e.g. Hypromellose USP Pharmacoat-603, hydroxypropylmethylcellulose acetate stearate (Agoate HS-LF and HS), hydroxyethylcellulose, hydroxypropylcellulose (e.g., Klucel®), ethylcellulose (e.g., Ethocel®), and microcrystalline cellulose (e.g., Avicel®), microcrystalline dextrose, amylose, magnesium aluminum silicate, polysaccharide acids, bentonites, gelatin, polyvinylpyrrolidone/vinyl acetate copolymer, crospovidone, povidone, starch, pregelatinized starch, tragacanth, dextrin, a sugar, such as sucrose (e.g., Dipac®), glucose, dextrose, molasses, mannitol, sorbitol, xylitol (e.g., Xylitab®), lactose, a natural or synthetic gum such as acacia, tragacanth, ghatti gum, mucilage of isapol husks, starch, polyvinylpyrrolidone (e.g., Povidone® CL, Kollidon® CL, Polyplasdone® XL-10, and Povidone® K-12), larch arabogalactan, Veegum®, polyethylene glycol, waxes, sodium alginate, and the like.

In general, binder levels of 20-70% are used in powder-filled gelatin capsule formulations. Binder usage level in tablet formulations varies whether direct compression, wet granulation, roller compaction, or usage of other excipients such as fillers which itself can act as moderate binder. Formulators skilled in art can determine the binder level for the formulations, but binder usage level of up to 70% in tablet formulations is common.

Suitable lubricants or glidants for use in the solid dosage forms described herein include, but are not limited to, stearic acid, calcium hydroxide, talc, corn starch, sodium stearyl fumerate, alkali-metal and alkaline earth metal salts, such as aluminum, calcium, magnesium, zinc, stearic acid, sodium stearates, magnesium stearate, zinc stearate, waxes, Stearowet®, boric acid, sodium benzoate, sodium acetate, sodium chloride, leucine, a polyethylene glycol or a methoxypolyethylene glycol such as Carbowax™, PEG 4000, PEG 5000, PEG 6000, propylene glycol, sodium oleate, glyceryl behenate, glyceryl palmitostearate, glyceryl benzoate, magnesium or sodium lauryl sulfate, and the like.

Suitable diluents for use in the solid dosage forms described herein include, but are not limited to, sugars (including lactose, sucrose, and dextrose), polysaccharides (including dextrates and maltodextrin), polyols (including mannitol, xylitol, and sorbitol), cyclodextrins and the like.

The term “non water-soluble diluent” represents compounds typically used in the formulation of pharmaceuticals, such as calcium phosphate, calcium sulfate, starches, modified starches and microcrystalline cellulose, and microcellulose (e.g., having a density of about 0.45 g/cm³, e.g. Avicel, powdered cellulose), and talc.

Suitable wetting agents for use in the solid dosage forms described herein include, for example, oleic acid, glyceryl monostearate, sorbitan monooleate, sorbitan monolaurate, triethanolamine oleate, polyoxyethylene sorbitan monooleate, polyoxyethylene sorbitan monolaurate, quaternary ammonium compounds (e.g., Polyquat 10®), sodium oleate, sodium lauryl sulfate, magnesium stearate, sodium docusate, triacetin, vitamin E TPGS and the like.

Suitable surfactants for use in the solid dosage forms described herein include, for example, sodium lauryl sulfate, sorbitan monooleate, polyoxyethylene sorbitan monooleate, polysorbates, polaxomers, bile salts, glyceryl monostearate, copolymers of ethylene oxide and propylene oxide, e.g., Pluronic® (BASF), and the like.

Suitable suspending agents for use in the solid dosage forms described here include, but are not limited to, polyvinylpyrrolidone, e.g., polyvinylpyrrolidone K12, polyvinylpyrrolidone K17, polyvinylpyrrolidone K25, or polyvinylpyrrolidone K30, polyethylene glycol, e.g., the polyethylene glycol can have a molecular weight of about 300 to about 6000, or about 3350 to about 4000, or about 7000 to about 5400, vinyl pyrrolidone/vinyl acetate copolymer (S630), sodium carboxymethylcellulose, methylcellulose, hydroxy-propylmethylcellulose, polysorbate-80, hydroxyethylcellulose, sodium alginate, gums, such as, e.g., gum tragacanth and gum acacia, guar gum, xanthans, including xanthan gum, sugars, cellulosics, such as, e.g., sodium carboxymethylcellulose, methylcellulose, sodium carboxymethylcellulose, hydroxypropylmethylcellulose, hydroxyethylcellulose, polysorbate-80, sodium alginate, polyethoxylated sorbitan monolaurate, polyethoxylated sorbitan monolaurate, povidone and the like.

Suitable antioxidants for use in the solid dosage forms described herein include, for example, e.g., butylated hydroxytoluene (BHT), sodium ascorbate, and tocopherol.

It should be appreciated that there is considerable overlap between additives used in the solid dosage forms described herein. Thus, the above-listed additives should be taken as merely exemplary, and not limiting, of the types of additives that can be included in solid dosage forms described herein. The amounts of such additives can be readily determined by one skilled in the art, according to the particular properties desired.

In some embodiments, one or more layers of the pharmaceutical composition are plasticized. Illustratively, a plasticizer is generally a high boiling point solid or liquid. Suitable plasticizers can be added from about 0.01% to about 50% by weight (w/w) of the coating composition. Plasticizers include, but are not limited to, diethyl phthalate, citrate esters, polyethylene glycol, glycerol, acetylated glycerides, triacetin, polypropylene glycol, polyethylene glycol, triethyl citrate, dibutyl sebacate, stearic acid, stearol, stearate, and castor oil.

Compressed tablets are solid dosage forms prepared by compacting the bulk blend of the formulations described above. In various embodiments, compressed tablets which are designed to dissolve in the mouth will include one or more flavoring agents. In some embodiments, the compressed tablets will include a film surrounding the final compressed tablet. In some embodiments, the film coating can provide a delayed release of the compound of any of Formula (I), (I-1), (I-1a), (I-1b), (I-2), (I-2a), (I-2b), (I-3), (I-4), or (I-5) from the formulation. In some embodiments, the film coating aids in patient compliance (e.g., Opadry® coatings or sugar coating). Film coatings including Opadry® typically range from about 1% to about 3% of the tablet weight. In some embodiments, the compressed tablets include one or more excipients.

A capsule may be prepared, for example, by placing the bulk blend of the formulation of the compound of any of Formula (I), (I-1), (I-1a), (I-1b), (I-2), (I-2a), (I-2b), (I-3), (I-4), or (I-5), described above, inside of a capsule. In some embodiments, the formulations (non-aqueous suspensions and solutions) are placed in a soft gelatin capsule. In some embodiments, the formulations are placed in standard gelatin capsules or non-gelatin capsules such as capsules comprising HPMC. In some embodiments, the formulation is placed in a sprinkle capsule, wherein the capsule may be swallowed whole or the capsule may be opened and the contents sprinkled on food prior to eating. In some embodiments, the therapeutic dose is split into multiple (e.g., two, three, or four) capsules. In some embodiments, the entire dose of the formulation is delivered in a capsule form.

In various embodiments, the particles of the compound of any of Formula (I), (I-1), (I-1a), (I-1b), (I-2), (I-2a), (I-2b), (I-3), (I-4), or (I-5) and one or more excipients are dry blended and compressed into a mass, such as a tablet, having a hardness sufficient to provide a pharmaceutical composition that substantially disintegrates within less than about 30 minutes, less than about 35 minutes, less than about 40 minutes, less than about 45 minutes, less than about 50 minutes, less than about 55 minutes, or less than about 60 minutes, after oral administration, thereby releasing the formulation into the gastrointestinal fluid.

In some embodiments, dosage forms may include microencapsulated formulations. In some embodiments, one or more other compatible materials are present in the microencapsulation material. Exemplary materials include, but are not limited to, pH modifiers, erosion facilitators, anti-foaming agents, antioxidants, flavoring agents, and carrier materials such as binders, suspending agents, disintegration agents, filling agents, surfactants, solubilizers, stabilizers, lubricants, wetting agents, and diluents.

Materials useful for the microencapsulation described herein include materials compatible with compounds of any of Formula (I), (I-1), (I-1a), (I-1b), (I-2), (I-2a), (I-2b), (I-3), (I-4), or (I-5) which sufficiently isolate the compound of any of Formula (I), (I-1), (I-1a), (I-1b), (I-2), (I-2a), (I-2b), (I-4), or (I-5) from other non-compatible excipients. Materials compatible with compounds of any of Formula (I), (I-1), (I-1a), (I-1b), (I-2), (I-2a), (I-2b), (I-3), (I-4), or (I-5) are those that delay the release of the compounds of any of Formula (I), (I-1), (I-1a), (I-1b), (I-2), (I-2a), (I-2b), (I-3), (I-4), or (I-5), in vivo.

Exemplary microencapsulation materials useful for delaying the release of the formulations including compounds described herein, include, but are not limited to, hydroxypropyl cellulose ethers (HPC) such as Klucel® or Nisso HPC, low-substituted hydroxypropyl cellulose ethers (L-HPC), hydroxypropyl methyl cellulose ethers (HPMC) such as Seppifilm-LC, Pharmacoat®, Metolose SR, Methocel®-E, Opadry YS, PrimaFlo, Benecel MP824, and Benecel MP843, methylcellulose polymers such as Methocel®-A, hydroxypropylmethylcellulose acetate stearate Aqoat (HF-LS, IF-LG, HF-MS) and Metolose®, Ethylcelluloses (EC) and mixtures thereof such as E461, Ethocel®, Aqualon®-EC, Surelease®, Polyvinyl alcohol (PVA) such as Opadry AMB, hydroxyethylcelluloses such as Natrosol®, carboxymethylcelluloses and salts of carboxymethylcelluloses (CMC) such as Aqualon®-CMC, polyvinyl alcohol and polyethylene glycol co-polymers such as Kollicoat IR®, monoglycerides (Myverol), triglycerides (KLX), polyethylene glycols, modified food starch, acrylic polymers and mixtures of acrylic polymers with cellulose ethers such as Eudragit® EPO, Eudragit® L30D-55, Eudragit® FS 30D Eudragit® L100-55, Eudragit® L100, Eudragit® S100, Eudragit® RD100, Eudragit® E100, Eudragit® L12.5, Eudragit® S12.5, Eudragit® NE30D, and Eudragit® NE 40D, cellulose acetate phthalate, sepifilms such as mixtures of HPMC and stearic acid, cyclodextrins, and mixtures of these materials.

In some embodiments, plasticizers such as polyethylene glycols, e.g., PEG 300, PEG 400, PEG 600, PEG 1450, PEG 3350, and PEG 800, stearic acid, propylene glycol, oleic acid, and triacetin are incorporated into the microencapsulation material. In some embodiments, the microencapsulating material useful for delaying the release of the pharmaceutical compositions is from the USP or the National Formulary (NF). In some embodiments, the microencapsulation material is Klucel. In some embodiments, the microencapsulation material is methocel.

Microencapsulated compounds of any of Formula (I), (I-1), (I-1a), (I-1b), (I-2), (I-2a), (I-2b), (I-3), (I-4), or (I-5) may be formulated by methods known by one of ordinary skill in the art. Such known methods include, e.g., spray drying processes, spinning disk-solvent processes, hot melt processes, spray chilling methods, fluidized bed, electrostatic deposition, centrifugal extrusion, rotational suspension separation, polymerization at liquid-gas or solid-gas interface, pressure extrusion, or spraying solvent extraction bath. In addition to these, several chemical techniques, e.g., complex coacervation, solvent evaporation, polymer-polymer incompatibility, interfacial polymerization in liquid media, in situ polymerization, in-liquid drying, and desolvation in liquid media could also be used. Furthermore, other methods such as roller compaction, extrusion/spheronization, coacervation, or nanoparticle coating may also be used.

In some embodiments, the particles of compounds of any of Formula (I), (I-1), (I-1a), (I-1b), (I-2), (I-2a), (I-2b), (I-3), (I-4), or (I-5) are microencapsulated prior to being formulated into one of the above forms. In still some embodiments, some or most of the particles are coated prior to being further formulated by using standard coating procedures, such as those described in Remington's Pharmaceutical Sciences, 20th Edition (2000).

In some embodiments, the solid dosage formulations of the compounds of any of Formula (I), (I-1), (I-1a), (I-1b), (I-2), (I-2a), (I-2b), (I-3), (I-4), or (I-5) are plasticized (coated) with one or more layers. Illustratively, a plasticizer is generally a high boiling point solid or liquid. Suitable plasticizers can be added from about 0.01% to about 50% by weight (w/w) of the coating composition. Plasticizers include, but are not limited to, diethyl phthalate, citrate esters, polyethylene glycol, glycerol, acetylated glycerides, triacetin, polypropylene glycol, polyethylene glycol, triethyl citrate, dibutyl sebacate, stearic acid, stearol, stearate, and castor oil.

In some embodiments, a powder including the formulations with a compound of any of Formula (I), (I-1), (I-1a), (I-1b), (I-2), (I-2a), (I-2b), (I-3), (I-4), or (I-5), described herein, may be formulated to include one or more pharmaceutical excipients and flavors. Such a powder may be prepared, for example, by mixing the formulation and optional pharmaceutical excipients to form a bulk blend composition. Additional embodiments also include a suspending agent and/or a wetting agent. This bulk blend is uniformly subdivided into unit dosage packaging or multi-dosage packaging units.

In still some embodiments, effervescent powders are also prepared in accordance with the present disclosure. Effervescent salts have been used to disperse medicines in water for oral administration. Effervescent salts are granules or coarse powders containing a medicinal agent in a dry mixture, usually composed of sodium bicarbonate, citric acid and/or tartaric acid. When salts of the compositions described herein are added to water, the acids and the base react to liberate carbon dioxide gas, thereby causing “effervescence.” Examples of effervescent salts include, e.g., the following ingredients: sodium bicarbonate or a mixture of sodium bicarbonate and sodium carbonate, citric acid and/or tartaric acid. Any acid-base combination that results in the liberation of carbon dioxide can be used in place of the combination of sodium bicarbonate and citric and tartaric acids, as long as the ingredients were suitable for pharmaceutical use and result in a pH of about 6.0 or higher.

In some embodiments, the formulations described herein, which include a compound of Formula (I), (I-1), (I-1a), (I-1b), (I-2), (I-2a), (I-2b), (I-3), (I-4), or (I-5), are solid dispersions. Methods of producing such solid dispersions are known in the art and include, but are not limited to, for example, U.S. Pat. Nos. 4,343,789, 5,340,591, 5,456,923, 5,700,485, 5,723,269, and U.S. Pub. Appl 2004/0013734, each of which is specifically incorporated by reference. In some embodiments, the formulations described herein are solid solutions. Solid solutions incorporate a substance together with the active agent and other excipients such that heating the mixture results in dissolution of the drug and the resulting composition is then cooled to provide a solid blend which can be further formulated or directly added to a capsule or compressed into a tablet. Methods of producing such solid solutions are known in the art and include, but are not limited to, for example, U.S. Pat. Nos. 4,151,273, 5,281,420, and 6,083,518, each of which is specifically incorporated by reference.

The pharmaceutical solid oral dosage forms including formulations described herein, which include a compound of any of Formula (I), (I-1), (I-1a), (I-1b), (I-2), (I-2a), (I-2b), (I-3), (I-4), or (I-5) can be further formulated to provide a controlled release of the compound of Formula (I). Controlled release refers to the release of the compound of any of Formula (I), (I-1), (I-1a), (I-1b), (I-2), (I-2a), (I-2b), (I-3), (I-4), or (I-5) from a dosage form in which it is incorporated according to a desired profile over an extended period of time. Controlled release profiles include, for example, sustained release, prolonged release, pulsatile release, and delayed release profiles. In contrast to immediate release compositions, controlled release compositions allow delivery of an agent to a subject over an extended period of time according to a predetermined profile. Such release rates can provide therapeutically effective levels of agent for an extended period of time and thereby provide a longer period of pharmacologic response while minimizing side effects as compared to conventional rapid release dosage forms. Such longer periods of response provide for many inherent benefits that are not achieved with the corresponding short acting, immediate release preparations.

In some embodiments, the solid dosage forms described herein can be formulated as enteric coated delayed release oral dosage forms, i.e., as an oral dosage form of a pharmaceutical composition as described herein which utilizes an enteric coating to affect release in the small intestine of the gastrointestinal tract. The enteric coated dosage form may be a compressed or molded or extruded tablet/mold (coated or uncoated) containing granules, powder, pellets, beads or particles of the active ingredient and/or other composition components, which are themselves coated or uncoated. The enteric coated oral dosage form may also be a capsule (coated or uncoated) containing pellets, beads or granules of the solid carrier or the composition, which are themselves coated or uncoated.

The term “delayed release” as used herein refers to the delivery so that the release can be accomplished at some generally predictable location in the intestinal tract more distal to that which would have been accomplished if there had been no delayed release alterations. In some embodiments the method for delay of release is coating. Any coatings should be applied to a sufficient thickness such that the entire coating does not dissolve in the gastrointestinal fluids at pH below about 5, but does dissolve at pH about 5 and above. It is expected that any anionic polymer exhibiting a pH-dependent solubility profile can be used as an enteric coating in the methods and compositions described herein to achieve delivery to the lower gastrointestinal tract. In some embodiments the polymers described herein are anionic carboxylic polymers. In some embodiments, the polymers and compatible mixtures thereof, and some of their properties, include, but are not limited to:
Shellac, also called purified lac, a refined product obtained from the resinous secretion of an insect. This coating dissolves in media of pH>7;
Acrylic polymers. The performance of acrylic polymers (primarily their solubility in biological fluids) can vary based on the degree and type of substitution. Examples of suitable acrylic polymers include methacrylic acid copolymers and ammonium methacrylate copolymers. The Eudragit series E, L, S, RL, RS and NE (Rohm Pharma) are available as solubilized in organic solvent, aqueous dispersion, or dry powders. The Eudragit series RL, NE, and RS are insoluble in the gastrointestinal tract but are permeable and are used primarily for colonic targeting. The Eudragit series E dissolve in the stomach. The Eudragit series L, L-30D and S are insoluble in stomach and dissolve in the intestine;
Cellulose Derivatives. Examples of suitable cellulose derivatives are: ethyl cellulose; reaction mixtures of partial acetate esters of cellulose with phthalic anhydride. The performance can vary based on the degree and type of substitution. Cellulose acetate phthalate (CAP) dissolves in pH>6. Aquateric (FMC) is an aqueous based system and is a spray dried CAP pseudolatex with particles <1 μm. Other components in Aquateric can include pluronics, Tweens, and acetylated monoglycerides. Other suitable cellulose derivatives include: cellulose acetate trimellitate (Eastman); methylcellulose (Pharmacoat, Methocel); hydroxypropylmethyl cellulose phthalate (HPMCP); hydroxypropylmethyl cellulose succinate (HPMCS); and hydroxypropylmethylcellulose acetate succinate (e.g., AQOAT (Shin Etsu)). The performance can vary based on the degree and type of substitution. For example, HPMCP such as, HP-50, HP-55, HP-555, HP-55F grades are suitable. The performance can vary based on the degree and type of substitution. For example, suitable grades of hydroxypropylmethylcellulose acetate succinate include, but are not limited to, AS-LG (LF), which dissolves at pH 5, AS-MG (MF), which dissolves at pH 5.5, and AS-HG (HF), which dissolves at higher pH. These polymers are offered as granules, or as fine powders for aqueous dispersions;
Poly Vinyl Acetate Phthalate (PVAP). PVAP dissolves in pH>5, and it is much less permeable to water vapor and gastric fluids.

In some embodiments, the coating can, and usually does, contain a plasticizer and possibly other coating excipients such as colorants, talc, and/or magnesium stearate, which are well known in the art. Suitable plasticizers include triethyl citrate (Citroflex 2), triacetin (glyceryl triacetate), acetyl triethyl citrate (Citroflec A2), Carbowax 400 (polyethylene glycol 400), diethyl phthalate, tributyl citrate, acetylated monoglycerides, glycerol, fatty acid esters, propylene glycol, and dibutyl phthalate. In particular, anionic carboxylic acrylic polymers usually will contain 10-25% by weight of a plasticizer, especially dibutyl phthalate, polyethylene glycol, triethyl citrate and triacetin. Conventional coating techniques such as spray or pan coating are employed to apply coatings. The coating thickness must be sufficient to ensure that the oral dosage form remains intact until the desired site of topical delivery in the intestinal tract is reached.

Colorants, detackifiers, surfactants, antifoaming agents, lubricants (e.g., carnuba wax or PEG) may be added to the coatings besides plasticizers to solubilize or disperse the coating material, and to improve coating performance and the coated product.

In some embodiments, the formulations described herein, which include a compound of Formula (I), (I-1), (I-1a), (I-1b), (I-2), (I-2a), (I-2b), (I-3), (I-4), or (I-5), are delivered using a pulsatile dosage form. A pulsatile dosage form is capable of providing one or more immediate release pulses at predetermined time points after a controlled lag time or at specific sites. Pulsatile dosage forms including the formulations described herein, which include a compound of any of Formula (I), (I-1), (I-1a), (I-1b), (I-2), (I-2a), (I-2b), (I-3), (I-4), or (I-5) may be administered using a variety of pulsatile formulations known in the art. For example, such formulations include, but are not limited to, those described in U.S. Pat. Nos. 5,011,692, 5,017,381, 5,229,135, and 5,840,329, each of which is specifically incorporated by reference. Other pulsatile release dosage forms suitable for use with the present formulations include, but are not limited to, for example, U.S. Pat. Nos. 4,871,549, 5,260,068, 5,260,069, 5,508,040, 5,567,441 and 5,837,284, all of which are specifically incorporated by reference. In some embodiments, the controlled release dosage form is pulsatile release solid oral dosage form including at least two groups of particles, (i.e. multiparticulate) each containing the formulation described herein. The first group of particles provides a substantially immediate dose of the compound of any of Formula (I), (I-1), (I-1a), (I-1b), (I-2), (I-2a), (I-2b), (I-3), (I-4), or (I-5) upon ingestion by a mammal. The first group of particles can be either uncoated or include a coating and/or sealant. The second group of particles includes coated particles, which includes from about 2% to about 75%, from about 2.5% to about 70%, or from about 40% to about 70%, by weight of the total dose of the compound of any of Formula (I), (I-1), (I-1a), (I-1b), (I-2), (I-2a), (I-2b), (I-3), (I-4), or (I-5) in said formulation, in admixture with one or more binders. The coating includes a pharmaceutically acceptable ingredient in an amount sufficient to provide a delay of from about 2 hours to about 7 hours following ingestion before release of the second dose. Suitable coatings include one or more differentially degradable coatings such as, by way of example only, pH sensitive coatings (enteric coatings) such as acrylic resins (e.g., Eudragit® EPO, Eudragit® L30D-55, Eudragit® FS 30D Eudragit® L100-55, Eudragit® L100, Eudragit® S100, Eudragit® RD100, Eudragit® E100, Eudragit® L12.5, Eudragit® S12.5, and Eudragit® NE30D, Eudragit® NE 40D®) either alone or blended with cellulose derivatives, e.g., ethylcellulose, or non-enteric coatings having variable thickness to provide differential release of the formulation that includes a compound of any of Formula (I).

Many other types of controlled release systems known to those of ordinary skill in the art and are suitable for use with the formulations described herein. Examples of such delivery systems include, e.g., polymer-based systems, such as polylactic and polyglycolic acid, plyanhydrides and polycaprolactone; porous matrices, nonpolymer-based systems that are lipids, including sterols, such as cholesterol, cholesterol esters and fatty acids, or neutral fats, such as mono-, di- and triglycerides; hydrogel release systems; silastic systems; peptide-based systems; wax coatings, bioerodible dosage forms, compressed tablets using conventional binders and the like. See, e.g., Liberman et al., Pharmaceutical Dosage Forms, 2 Ed., Vol. 1, pp. 209-214 (1990); Singh et al., Encyclopedia of Pharmaceutical Technology, 2^{nd} Ed., pp. 751-753 (2002); U.S. Pat. Nos. 4,327,725, 4,624,848, 4,968,509, 5,461,140, 5,456,923, 5,516,527, 5,622,721, 5,686,105, 5,700,410, 5,977,175, 6,465,014 and 6,932,983, each of which is specifically incorporated by reference.

In some embodiments, pharmaceutical compositions are provided that include particles of the compounds of any of Formula (I), (I-1), (I-1a), (I-1b), (I-2), (I-2a), (I-2b), (I-4), or (I-5), described herein and at least one dispersing agent or suspending agent for oral administration to a subject. The formulations may be a powder and/or granules for suspension, and upon admixture with water, a substantially uniform suspension is obtained.

Liquid formulation dosage forms for oral administration can be aqueous suspensions selected from the group including, but not limited to, pharmaceutically acceptable aqueous oral dispersions, emulsions, solutions, elixirs, gels, and syrups. See, e.g., Singh et al., Encyclopedia of Pharmaceutical Technology, 2^{nd} Ed., pp. 754-757 (2002). In addition to the particles of compound of Formula (A), the liquid dosage forms may include additives, such as: (a) disintegrating agents; (b) dispersing agents; (c) wetting agents; (d) at least one preservative, (e) viscosity enhancing agents, (f) at least one sweetening agent, and (g) at least one flavoring agent. In some embodiments, the aqueous dispersions can further include a crystalline inhibitor.

The aqueous suspensions and dispersions described herein can remain in a homogenous state, as defined in The USP Pharmacists' Pharmacopeia (2005 edition, chapter 905), for at least 4 hours. The homogeneity should be determined by a sampling method consistent with regard to determining homogeneity of the entire composition. In some embodiments, an aqueous suspension can be re-suspended into a homogenous suspension by physical agitation lasting less than 1 minute. In some embodiments, an aqueous suspension can be re-suspended into a homogenous suspension by physical agitation lasting less than 45 seconds. In yet some embodiments, an aqueous suspension can be re-suspended into a homogenous suspension by physical agitation lasting less than 30 seconds. In still some embodiments, no agitation is necessary to maintain a homogeneous aqueous dispersion.

Examples of disintegrating agents for use in the aqueous suspensions and dispersions include, but are not limited to, a starch, e.g., a natural starch such as corn starch or potato starch, a pregelatinized starch such as National 1551 or Amijel®, or sodium starch glycolate such as Promogel® or Explotab®; a cellulose such as a wood product, methylcrystalline cellulose, e.g., Avicel®, Avicel® PH101, Avicel® PH102, Avicel® PH105, Elcema® P100, Emcocel®, Vivacel®, Ming Tia®, and Solka-Floc®, methylcellulose, croscarmellose, or a cross-linked cellulose, such as cross-linked sodium carboxymethylcellulose (Ac-Di-Sol®), cross-linked carboxymethylcellulose, or cross-linked croscarmellose; a cross-linked starch such as sodium starch glycolate; a cross-linked polymer such as crospovidone; a cross-linked polyvinylpyrrolidone; alginate such as alginic acid or a salt of alginic acid such as sodium alginate; a clay such as Veegum® HV (magnesium aluminum silicate); a gum such as agar, guar, locust bean, Karaya, pectin, or tragacanth; sodium starch glycolate; bentonite; a natural sponge; a surfactant; a resin such as a cation-exchange resin; citrus pulp; sodium lauryl sulfate; sodium lauryl sulfate in combination starch; and the like.

In some embodiments, the dispersing agents suitable for the aqueous suspensions and dispersions described herein are known in the art and include, for example, hydrophilic polymers, electrolytes, Tween® 60 or 80, PEG, polyvinylpyrrolidone (PVP; commercially known as Plasdone®), and the carbohydrate-based dispersing agents such as, for example, hydroxypropylcellulose and hydroxypropyl cellulose ethers (e.g., HPC, HPC-SL, and HPC-L), hydroxypropyl methylcellulose and hydroxypropyl methylcellulose ethers (e.g. HPMC K100, HPMC K4M, HPMC K15M, and HPMC K100M), carboxymethylcellulose sodium, methylcellulose, hydroxyethylcellulose, hydroxypropylmethyl-cellulose phthalate, hydroxypropylmethyl-cellulose acetate stearate, noncrystalline cellulose, magnesium aluminum silicate, triethanolamine, polyvinyl alcohol (PVA), polyvinylpyrrolidone/vinyl acetate copolymer (Plasdone®, e.g., S-630), 4-(1,1,3,3-tetramethylbutyl)-phenol polymer with ethylene oxide and formaldehyde (also known as tyloxapol), poloxamers (e.g., Pluronics F68®, F88®, and F108®, which are block copolymers of ethylene oxide and propylene oxide); and poloxamines (e.g., Tetronic 908®, also known as Poloxamine 908®, which is a tetrafunctional block copolymer derived from sequential addition of propylene oxide and ethylene oxide to ethylenediamine (BASF Corporation, Parsippany, N.J.)). In some embodiments, the dispersing agent is selected from a group not comprising one of the following agents: hydrophilic polymers; electrolytes; Tween® 60 or 80; PEG; polyvinylpyrrolidone (PVP); hydroxypropylcellulose and hydroxypropyl cellulose ethers (e.g., HPC, HPC-SL, and HPC-L); hydroxypropyl methylcellulose and hydroxypropyl methylcellulose ethers (e.g. HPMC K100, HPMC K4M, HPMC K15M, HPMC K100M, and Pharmacoat® USP 2910 (Shin-Etsu)); carboxymethylcellulose sodium; methylcellulose; hydroxyethylcellulose; hydroxypropylmethyl-cellulose phthalate; hydroxypropylmethyl-cellulose acetate stearate; non-crystalline cellulose; magnesium aluminum silicate; triethanolamine; polyvinyl alcohol (PVA); 4-(1,1,3,3-tetramethylbutyl)-phenol polymer with ethylene oxide and formaldehyde; poloxamers (e.g., Pluronics F68®, F88®, and F108®, which are block copolymers of ethylene oxide and propylene oxide); or poloxamines (e.g., Tetronic 908®, also known as Poloxamine 908®).

Wetting agents suitable for the aqueous suspensions and dispersions described herein are known in the art and include, but are not limited to, cetyl alcohol, glycerol monostearate, polyoxyethylene sorbitan fatty acid esters (e.g., the commercially available Tweens® such as e.g., Tween 20® and Tween 80® (ICI Specialty Chemicals)), and polyethylene glycols (e.g., Carbowaxs 3350® and 1450®, and Carbopol 934® (Union Carbide)), oleic acid, glyceryl monostearate, sorbitan monooleate, sorbitan monolaurate, triethanolamine oleate, polyoxyethylene sorbitan monooleate, polyoxyethylene sorbitan monolaurate, sodium oleate, sodium lauryl sulfate, sodium docusate, triacetin, vitamin E TPGS, sodium taurocholate, simethicone, phosphotidylcholine and the like

Suitable preservatives for the aqueous suspensions or dispersions described herein include, for example, potassium sorbate, parabens (e.g., methylparaben and propylparaben), benzoic acid and its salts, other esters of parahydroxybenzoic acid such as butylparaben, alcohols such as ethyl alcohol or benzyl alcohol, phenolic compounds such as phenol, or quaternary compounds such as benzalkonium chloride. Preservatives, as used herein, are incorporated into the dosage form at a concentration sufficient to inhibit microbial growth.

Suitable viscosity enhancing agents for the aqueous suspensions or dispersions described herein include, but are not limited to, methyl cellulose, xanthan gum, carboxymethyl cellulose, hydroxypropyl cellulose, hydroxypropylmethyl cellulose, Plasdon® S-630, carbomer, polyvinyl alcohol, alginates, acacia, chitosans and combinations thereof. The concentration of the viscosity enhancing agent will depend upon the agent selected and the viscosity desired.

Examples of sweetening agents suitable for the aqueous suspensions or dispersions described herein include, for example, acacia syrup, acesulfame K, alitame, anise, apple, aspartame, banana, Bavarian cream, berry, black currant, butterscotch, calcium citrate, camphor, caramel, cherry, cherry cream, chocolate, cinnamon, bubble gum, citrus, citrus punch, citrus cream, cotton candy, cocoa, cola, cool cherry, cool citrus, cyclamate, cylamate, dextrose, eucalyptus, eugenol, fructose, fruit punch, ginger, glycyrrhetinate, glycyrrhiza (licorice) syrup, grape, grapefruit, honey, isomalt, lemon, lime, lemon cream, monoammonium glyrrhizinate (MagnaSweet®), maltol, mannitol, maple, marshmallow, menthol, mint cream, mixed berry, neohesperidine DC, neotame, orange, pear, peach, peppermint, peppermint cream, Prosweet® Powder, raspberry, root beer, rum, saccharin, safrole, sorbitol, spearmint, spearmint cream, strawberry, strawberry cream, stevia, sucralose, sucrose, sodium saccharin, saccharin, aspartame, acesulfame potassium, mannitol, talin, sucralose, sorbitol, swiss cream, tagatose, tangerine, thaumatin, tutti fruitti, vanilla, walnut, watermelon, wild cherry, wintergreen, xylitol, or any combination of these flavoring ingredients, e.g., anise-menthol, cherry-anise, cinnamon-orange, cherry-cinnamon, chocolate-mint, honey-lemon, lemon-lime, lemon-mint, menthol-eucalyptus, orange-cream, vanilla-mint, and mixtures thereof. In some embodiments, the aqueous liquid dispersion can comprise a sweetening agent or flavoring agent in a concentration ranging from about 0.001% to about 1.0% the volume of the aqueous dispersion. In some embodiments, the aqueous liquid dispersion can comprise a sweetening agent or flavoring agent in a concentration ranging from about 0.005% to about 0.5% the volume of the aqueous dispersion. In yet some embodiments, the aqueous liquid dispersion can comprise a sweetening agent or flavoring agent in a concentration ranging from about 0.01% to about 1.0% the volume of the aqueous dispersion.

In addition to the additives listed above, the liquid formulations can also include inert diluents commonly used in the art, such as water or other solvents, solubilizing agents, and emulsifiers. Exemplary emulsifiers are ethyl alcohol, isopropyl alcohol, ethyl carbonate, ethyl acetate, benzyl alcohol, benzyl benzoate, propyleneglycol, 1,3-butyleneglycol, dimethylformamide, sodium lauryl sulfate, sodium doccusate, cholesterol, cholesterol esters, taurocholic acid, phosphotidylcholine, oils, such as cottonseed oil, groundnut oil, corn germ oil, olive oil, castor oil, and sesame oil, glycerol, tetrahydrofurfuryl alcohol, polyethylene glycols, fatty acid esters of sorbitan, or mixtures of these substances, and the like.

In some embodiments, the pharmaceutical compositions described herein can be self-emulsifying drug delivery systems (SEDDS). Emulsions are dispersions of one immiscible phase in another, usually in the form of droplets. Generally, emulsions are created by vigorous mechanical dispersion. SEDDS, as opposed to emulsions or microemulsions, spontaneously form emulsions when added to an excess of water without any external mechanical dispersion or agitation. An advantage of SEDDS is that only gentle mixing is required to distribute the droplets throughout the solution. Additionally, water or the aqueous phase can be added just prior to administration, which ensures stability of an unstable or hydrophobic active ingredient. Thus, the SEDDS provides an effective delivery system for oral and parenteral delivery of hydrophobic active ingredients. SEDDS may provide improvements in the bioavailability of hydrophobic active ingredients. Methods of producing self-emulsifying dosage forms are known in the art and include, but are not limited to, for example, U.S. Pat. Nos. 5,858,401, 6,667,048, and 6,960,563, each of which is specifically incorporated by reference.

It is to be appreciated that there is overlap between the above-listed additives used in the aqueous dispersions or suspensions described herein, since a given additive is often classified differently by different practitioners in the field, or is commonly used for any of several different functions. Thus, the above-listed additives should be taken as merely exemplary, and not limiting, of the types of additives that can be included in formulations described herein. The amounts of such additives can be readily determined by one skilled in the art, according to the particular properties desired.

### Intranasal Formulations

Intranasal formulations are known in the art and are described in, for example, U.S. Pat. Nos. 4,476,116, 5,116,817 and 6,391,452, each of which is specifically incorporated by reference. Formulations that include a compound of any of Formula (I), (I-1), (I-1a), (I-1b), (I-2), (I-2a), (I-2b), (I-3), (I-4), or (I-5) which are prepared according to these and other techniques well-known in the art are prepared as solutions in saline, employing benzyl alcohol or other suitable preservatives, fluorocarbons, and/or other solubilizing or dispersing agents known in the art. See, for example, Ansel, H. C. et al., Pharmaceutical Dosage Forms and Drug Delivery Systems, Sixth Ed. (1995). Preferably these compositions and formulations are prepared with suitable nontoxic pharmaceutically acceptable ingredients. These ingredients are known to those skilled in the preparation of nasal dosage forms and some of these can be found in REMINGTON: THE SCIENCE AND PRACTICE OF PHARMACY, 21st edition, 2005, a standard reference in the field. The choice of suitable carriers is highly dependent upon the exact nature of the nasal dosage form desired, e.g., solutions, suspensions, ointments, or gels. Nasal dosage forms generally contain large amounts of water in addition to the active ingredient. Minor amounts of other ingredients such as pH adjusters, emulsifiers or dispersing agents, preservatives, surfactants, gelling agents, or buffering and other stabilizing and solubilizing agents may also be present. The nasal dosage form should be isotonic with nasal secretions.

For administration by inhalation, the compounds of any of Formula (I), (I-1), (I-1a), (I-1b), (I-2), (I-2a), (I-2b), (I-3), (I-4), or (I-5), described herein may be in a form as an aerosol, a mist or a powder. Pharmaceutical compositions described herein are conveniently delivered in the form of an aerosol spray presentation from pressurized packs or a nebuliser, with the use of a suitable propellant, e.g., dichlorodifluoromethane, trichlorofluoromethane, dichlorotetrafluoroethane, carbon dioxide or other suitable gas. In the case of a pressurized aerosol, the dosage unit may be determined by providing a valve to deliver a metered amount. Capsules and cartridges of, such as, by way of example only, gelatin for use in an inhaler or insufflator may be formulated containing a powder mix of the compound described herein and a suitable powder base such as lactose or starch.

### Buccal Formulations

Buccal formulations that include compounds of any of Formula (I), (I-1), (I-1a), (I-1b), (I-2), (I-2a), (I-2b), (I-3), (I-4), or (I-5) may be administered using a variety of formulations known in the art. For example, such formulations include, but are not limited to, U.S. Pat. Nos. 4,229,447, 4,596,795, 4,755,386, and 5,739,136, each of which is specifically incorporated by reference. In addition, the buccal dosage forms described herein can further include a bioerodible (hydrolysable) polymeric carrier that also serves to adhere the dosage form to the buccal mucosa. The buccal dosage form is fabricated so as to erode gradually over a predetermined time period, wherein the delivery of the compound of any of Formula (I), (I-1), (I-1a), (I-1b), (I-2), (I-2a), (I-2b), (I-3), (I-4), or (I-5), is provided essentially throughout. Buccal drug delivery, as will be appreciated by those skilled in the art, avoids the disadvantages encountered with oral drug administration, e.g., slow absorption, degradation of the active agent by fluids present in the gastrointestinal tract and/or first-pass inactivation in the liver. With regard to the bioerodible (hydrolysable) polymeric carrier, it will be appreciated that virtually any such carrier can be used, so long as the desired drug release profile is not compromised, and the carrier is compatible with the compound of any of Formula (I), (I-1), (I-1a), (I-1b), (I-2), (I-2a), (I-2b), (I-3), (I-4), or (I-5), and any other components that may be present in the buccal dosage unit. Generally, the polymeric carrier comprises hydrophilic (water-soluble and water-swellable) polymers that adhere to the wet surface of the buccal mucosa. Examples of polymeric carriers useful herein include acrylic acid polymers and co, e.g., those known as “carbomers” (Carbopol®, which may be obtained from B.F. Goodrich, is one such polymer). Other components may also be incorporated into the buccal dosage forms described herein include, but are not limited to, disintegrants, diluents, binders, lubricants, flavoring, colorants, preservatives, and the like. For buccal or sublingual administration, the compositions may take the form of tablets, lozenges, or gels formulated in a conventional manner.

### Transdermal Formulations

Transdermal formulations described herein may be administered using a variety of devices which have been described in the art. For example, such devices include, but are not limited to, U.S. Pat. Nos. 3,598,122, 3,598,123, 3,710,795, 3,731,683, 3,742,951, 3,814,097, 3,921,636, 3,972,995, 3,993,072, 3,993,073, 3,996,934, 4,031,894, 4,060,084, 4,069,307, 4,077,407, 4,201,211, 4,230,105, 4,292,299, 4,292,303, 5,336,168, 5,665,378, 5,837,280, 5,869,090, 6,923,983, 6,929,801 and 6,946,144, each of which is specifically incorporated by reference in its entirety.

The transdermal dosage forms described herein may incorporate certain pharmaceutically acceptable excipients which are conventional in the art. In some embodiments, the transdermal formulations described herein include at least three components: (1) a formulation of a compound of any of Formula (I), (I-1), (I-1a), (I-1b), (I-2), (I-2a), (I-2b), (I-3), (I-4), or (I-5); (2) a penetration enhancer; and (3) an aqueous adjuvant. In addition, transdermal formulations can include additional components such as, but not limited to, gelling agents, creams and ointment bases, and the like. In some embodiments, the transdermal formulation can further include a woven or non-woven backing material to enhance absorption and prevent the removal of the transdermal formulation from the skin. In some embodiments, the transdermal formulations described herein can maintain a saturated or supersaturated state to promote diffusion into the skin.

Formulations suitable for transdermal administration of compounds described herein may employ transdermal delivery devices and transdermal delivery patches and can be lipophilic emulsions or buffered, aqueous solutions, dissolved and/or dispersed in a polymer or an adhesive. Such patches may be constructed for continuous, pulsatile, or on demand delivery of pharmaceutical agents. Still further, transdermal delivery of the compounds described herein can be accomplished by means of iontophoretic patches and the like. Additionally, transdermal patches can provide controlled delivery of the compounds of any of Formula (I), (I-1), (I-1a), (I-1b), (I-2), (I-2a), (I-2b), (I-3), (I-4), or (I-5). The rate of absorption can be slowed by using rate-controlling membranes or by trapping the compound within a polymer matrix or gel. Conversely, absorption enhancers can be used to increase absorption. An absorption enhancer or carrier can include absorbable pharmaceutically acceptable solvents to assist passage through the skin. For example, transdermal devices are in the form of a bandage comprising a backing member, a reservoir containing the compound optionally with carriers, optionally a rate controlling barrier to deliver the compound to the skin of the host at a controlled and predetermined rate over a prolonged period of time, and means to secure the device to the skin.

### Injectable Formulations

Formulations that include a compound of any of Formula (I), (I-1), (I-1a), (I-1b), (I-2), (I-2a), (I-2b), (I-3), (I-4), or (I-5), suitable for intramuscular, subcutaneous, or intravenous injection may include physiologically acceptable sterile aqueous or non-aqueous solutions, dispersions, suspensions or emulsions, and sterile powders for reconstitution into sterile injectable solutions or dispersions. Examples of suitable aqueous and non-aqueous carriers, diluents, solvents, or vehicles including water, ethanol, polyols (propyleneglycol, polyethylene-glycol, glycerol, cremophor and the like), suitable mixtures thereof, vegetable oils (such as olive oil) and injectable organic esters such as ethyl oleate. Proper fluidity can be maintained, for example, by the use of a coating such as lecithin, by the maintenance of the required particle size in the case of dispersions, and by the use of surfactants. Formulations suitable for subcutaneous injection may also contain additives such as preserving, wetting, emulsifying, and dispensing agents. Prevention of the growth of microorganisms can be ensured by various antibacterial and antifungal agents, such as parabens, chlorobutanol, phenol, sorbic acid, and the like. It may also be desirable to include isotonic agents, such as sugars, sodium chloride, and the like. Prolonged absorption of the injectable pharmaceutical form can be brought about by the use of agents delaying absorption, such as aluminum monostearate and gelatin.

For intravenous injections, compounds described herein may be formulated in aqueous solutions, preferably in physiologically compatible buffers such as Hank's solution, Ringer's solution, or physiological saline buffer. For transmucosal administration, penetrants appropriate to the barrier to be permeated are used in the formulation. Such penetrants are generally known in the art. For other parenteral injections, appropriate formulations may include aqueous or nonaqueous solutions, preferably with physiologically compatible buffers or excipients. Such excipients are generally known in the art.

Parenteral injections may involve bolus injection or continuous infusion. Formulations for injection may be presented in unit dosage form, e.g., in ampoules or in multi-dose containers, with an added preservative. The pharmaceutical composition described herein may be in a form suitable for parenteral injection as a sterile suspensions, solutions or emulsions in oily or aqueous vehicles, and may contain formulatory agents such as suspending, stabilizing and/or dispersing agents. Pharmaceutical compositions for parenteral administration include aqueous solutions of the active compounds in water-soluble form. Additionally, suspensions of the active compounds may be prepared as appropriate oily injection suspensions. Suitable lipophilic solvents or vehicles include fatty oils such as sesame oil, or synthetic fatty acid esters, such as ethyl oleate or triglycerides, or liposomes. Aqueous injection suspensions may contain substances which increase the viscosity of the suspension, such as sodium carboxymethyl cellulose, sorbitol, or dextran. Optionally, the suspension may also contain suitable stabilizers or agents which increase the solubility of the compounds to allow for the preparation of highly concentrated solutions. Alternatively, the active ingredient may be in powder form for constitution with a suitable vehicle, e.g., sterile pyrogen-free water, before use.

### Formulations

In certain embodiments, delivery systems for pharmaceutical compounds may be employed, such as, for example, liposomes and emulsions. In certain embodiments, compositions provided herein can also include an mucoadhesive polymer, selected from among, for example, carboxymethylcellulose, carbomer (acrylic acid polymer), poly(methylmethacrylate), polyacrylamide, polycarbophil, acrylic acid/butyl acrylate copolymer, sodium alginate and dextran.

In some embodiments, the compounds described herein may be administered topically and can be formulated into a variety of topically administrable compositions, such as solutions, suspensions, lotions, gels, pastes, medicated sticks, balms, creams or ointments. Such pharmaceutical compounds can contain solubilizers, stabilizers, tonicity enhancing agents, buffers and preservatives.

The compounds described herein may also be formulated in rectal compositions such as enemas, rectal gels, rectal foams, rectal aerosols, suppositories, jelly suppositories, or retention enemas, containing conventional suppository bases such as cocoa butter or other glycerides, as well as synthetic polymers such as polyvinylpyrrolidone, PEG, and the like. In suppository forms of the compositions, a low-melting wax such as, but not limited to, a mixture of fatty acid glycerides, optionally in combination with cocoa butter is first melted.

### Examples of Dosing and Treatment Regimens

The compounds described herein can be used in the preparation of medicaments for the treatment of diseases or conditions, for example those related to kidney disease and conditions. In addition, a method for treating any of the diseases or conditions described herein in a subject in need of such treatment, involves administration of pharmaceutical compositions containing at least one compound of any of Formula (I), (I-1), (I-1a), (I-1b), (I-2), (I-2a), (I-2b), (I-3), (I-4), or (I-5), described herein, or a pharmaceutically acceptable N-oxide, pharmaceutically active metabolite, or pharmaceutically acceptable solvate thereof, in therapeutically effective amounts to said subject.

The compositions containing the compound(s) described herein can be administered for prophylactic and/or therapeutic treatments. In therapeutic applications, the compositions are administered to a patient already suffering from a disease or condition, in an amount sufficient to cure or at least partially arrest the symptoms of the disease or condition. Amounts effective for this use will depend on the severity and course of the disease or condition, previous therapy, the patient's health status, weight, and response to the drugs, and the judgment of the treating physician. It is considered well within the skill of the art for one to determine such therapeutically effective amounts by routine experimentation (including, but not limited to, a dose escalation clinical trial).

In prophylactic applications, compositions containing the compounds described herein are administered to a patient susceptible to or otherwise at risk of a particular disease, disorder or condition. Such an amount is defined to be a “prophylactically effective amount or dose.” In this use, the precise amounts also depend on the patient's state of health, weight, and the like. It is considered well within the skill of the art for one to determine such prophylactically effective amounts by routine experimentation (e.g., a dose escalation clinical trial). When used in a patient, effective amounts for this use will depend on the severity and course of the disease, disorder or condition, previous therapy, the patient's health status and response to the drugs, and the judgment of the treating physician.

In the case wherein the patient's condition does not improve, upon the doctor's discretion the administration of the compounds may be administered chronically, that is, for an extended period of time, including throughout the duration of the patient's life in order to ameliorate or otherwise control or limit the symptoms of the patient's disease or condition.

In the case wherein the patient's status does improve, upon the doctor's discretion the administration of the compounds may be given continuously; alternatively, the dose of drug being administered may be temporarily reduced or temporarily suspended for a certain length of time (i.e., a “drug holiday”). The length of the drug holiday can vary between 2 days and 1 year, including by way of example only, 2 days, 3 days, 4 days, 5 days, 6 days, 7 days, 10 days, 12 days, 15 days, 20 days, 28 days, 35 days, 50 days, 70 days, 100 days, 120 days, 150 days, 180 days, 200 days, 250 days, 280 days, 300 days, 320 days, 350 days, or 365 days. The dose reduction during a drug holiday may be from 10%-100%, including, by way of example only, 10%, 15%, 20%, 25%, 30%, 35%, 40%, 45%, 50%, 55%, 60%, 65%, 70%, 75%, 80%, 85%, 90%, 95%, or 100%.

Once improvement of the patient's conditions has occurred, a maintenance dose is administered if necessary. Subsequently, the dosage or the frequency of administration, or both, can be reduced, as a function of the symptoms, to a level at which the improved disease, disorder or condition is retained. Patients can, however, require intermittent treatment on a long-term basis upon any recurrence of symptoms.

The amount of a given agent that will correspond to such an amount will vary depending upon factors such as the particular compound, disease or condition and its severity, the identity (e.g., weight) of the subject or host in need of treatment, but can nevertheless be routinely determined in a manner known in the art according to the particular circumstances surrounding the case, including, e.g., the specific agent being administered, the route of administration, the condition being treated, and the subject or host being treated. The desired dose may conveniently be presented in a single dose or as divided doses administered simultaneously (or over a short period of time) or at appropriate intervals, for example as two, three, four or more sub-doses per day.

The pharmaceutical composition described herein may be in unit dosage forms suitable for single administration of precise dosages. In unit dosage form, the formulation is divided into unit doses containing appropriate quantities of one or more compound. The unit dosage may be in the form of a package containing discrete quantities of the formulation. Non-limiting examples are packaged tablets or capsules, and powders in vials or ampoules. Aqueous suspension compositions can be packaged in single-dose non-reclosable containers. Alternatively, multiple-dose reclosable containers can be used, in which case it is typical to include a preservative in the composition. By way of example only, formulations for parenteral injection may be presented in unit dosage form, which include, but are not limited to ampoules, or in multi-dose containers, with an added preservative.

The foregoing ranges are merely suggestive, as the number of variables in regard to an individual treatment regime is large, and considerable excursions from these recommended values are not uncommon. Such dosages may be altered depending on a number of variables, not limited to the activity of the compound used, the disease or condition to be treated, the mode of administration, the requirements of the individual subject, the severity of the disease or condition being treated, and the judgment of the practitioner.

Toxicity and therapeutic efficacy of such therapeutic regimens can be determined by standard pharmaceutical procedures in cell cultures or experimental animals, including, but not limited to, the determination of the LD₅₀ (the dose lethal to 50% of the population) and the ED₅₀ (the dose therapeutically effective in 50% of the population). The dose ratio between the toxic and therapeutic effects is the therapeutic index and it can be expressed as the ratio between LD₅₀ and ED₅₀. Compounds exhibiting high therapeutic indices are preferred. The data obtained from cell culture assays and animal studies can be used in formulating a range of dosage for use in human. The dosage of such compounds lies preferably within a range of circulating concentrations that include the ED₅₀ with minimal toxicity. The dosage may vary within this range depending upon the dosage form employed and the route of administration utilized.

A compound of Formula (I), (I-1), (I-1a), (I-1b), (I-2), (I-2a), (I-2b), (I-3), (I-4), or (I-5) described herein can be used in the preparation of medicaments for the treatment of diseases or conditions. In addition, a method for treating any of the diseases or conditions described herein in a subject in need of such treatment, involves administration of pharmaceutical compositions containing a compound of Formula (I), (I-1), (I-1a), (I-1b), (I-2), (I-2a), (I-2b), (I-4), or (I-5) or a pharmaceutically active metabolite, or pharmaceutically acceptable solvate thereof, in therapeutically effective amounts to said subject.

In certain embodiments, the disease or condition is chronic kidney disease (CKD), diabetic nephropathy, IgA nephropathy, acute kidney failure, acute tubular necrosis, transplant related ischemia, acute kidney disease or acute kidney injury (AKI).

In one embodiment, the AKI is drug-induced AKI. In one embodiment, the AKI is drug-induced AKI is chemotherapy-induced AKI or immunotherapy-induced AKI.

In one embodiment, the drug-induced AKI is cisplatin induced AKI, aminoglycosides induced AKI, tenofovir induced AKI, contrast induced nephropathy, or PD-1/PDL-1 inhibitor-induced AKI.

In one embodiment, the drug-induced AKI is pembrolizumab (Keytruda)-induced AKI, durvalumab-induced AKI, atezolizumab-induced AKI, avelumab-induced AKI, nivolumab-induced AKI, durvalumab-induced AKI, cemiplimab-induced AKI, dostarlimab-induced AKI, and retifanlimab-induced AKI.

In one embodiment, the AKI is AKI after surgery for partial nephrectomy, AKI post kidney transplant, AKI in patients with dialysis, AKI after cardiac surgery, AKI in patients inside ICU, AKI caused by cirrhosis or sepsis, and AKI caused by hepatorenal syndrome

In a particular embodiment, the disease or condition is contrast induced-nephropathy.

In a particular embodiment, the disease or condition is Tenofovir-induced AKI.

In a particular embodiment, the disease or condition is aminoglycosides-induced AKI.

In a particular embodiment, the disease or condition is AKI after surgery.

In a particular embodiment, the disease or condition is AKI in patients with dialysis.

In a particular embodiment, the disease or condition is diabetic nephropathy.

In a more particular embodiment, the disease or condition is chronic kidney disease (CKD).

In some embodiments, the disease or condition is selected from:

| ACUTE | CHRONIC |
|---|---|
| Acute Kidney Injury (AKI) | Chronic Kidney Disease (CKD) |
| Acute tubular necrosis | IgA Nephropathy |
| Transplant related Ischemia | Diabetic Nephropathy |

In some embodiments, the disease or condition is selected from:

ACUTE KIDNEY INJURY/DISEASE

| Drug Induced | | Other causes |
|---|---|---|
| AKI | Post Surgery/Transplant | of AKI |
| Cisplatin | AKI after surgery for | Cirrhosis |
| induced AKI in | partial nephrectomy | |
| patients with | | |
| Head and Neck | | |
| Cancer | | |
| Aminoglycosides | AKI post Kidney | Hepatorenal |
| induced AKI in | transplant | syndrome |
| patients with | | |
| Cystic Fibrosis | | |
| Tenofovir | AKI for patients under | Sepsis |
| induced AKI in | dialysis | |
| patients with | | |
| Hep B and HIV | | |
| Contrast induced | | |
| AKI | | |
| PD-1/PDL-1 | | |
| inhibitor-induced | | |
| AKI | | |

### Kits/Articles of Manufacture

For use in the therapeutic applications described herein, kits and articles of manufacture are also described herein. Such kits can include a carrier, package, or container that is compartmentalized to receive one or more containers such as vials, tubes, and the like, each of the container(s) including one of the separate elements to be used in a method described herein. Suitable containers include, for example, bottles, vials, syringes, and test tubes. The containers can be formed from a variety of materials such as glass or plastic.

The articles of manufacture provided herein contain packaging materials. Packaging materials for use in packaging pharmaceutical products are well known to those of skill in the art. See, e.g., U.S. Pat. Nos. 5,323,907, 5,052,558 and 5,033,252. Examples of pharmaceutical packaging materials include, but are not limited to, blister packs, bottles, tubes, inhalers, pumps, bags, vials, containers, syringes, bottles, and any packaging material suitable for a selected formulation and intended mode of administration and treatment. A wide array of formulations of a compound of Formula (I), (I-1), (I-1a), (I-1b), (I-2), (I-2a), (I-2b), (I-4), or (I-5) and compositions provided herein are contemplated as are a variety of treatments for any disease, disorder, or condition that would benefit by using a compound of Formula (I), (I-1), (I-1a), (I-1b), (I-2), (I-2a), (I-2b), (I-4), or (I-5).

For example, the container(s) can include a compound of Formula (I), (I-1), (I-1a), (I-1b), (I-2), (I-2a), (I-2b), (I-4), or (I-5), optionally in a composition or in combination with another agent as disclosed herein. The container(s) optionally have a sterile access port (for example the container can be an intravenous solution bag or a vial having a stopper pierceable by a hypodermic injection needle). Such kits optionally comprising a compound with an identifying description or label or instructions relating to its use in the methods described herein.

A kit typically may include one or more additional containers, each with one or more of various materials (such as reagents, optionally in concentrated form, and/or devices) desirable from a commercial and user standpoint for use of a compound of Formula (I), (I-1), (I-1a), (I-1b), (I-2), (I-2a), (I-2b), (I-3), (I-4), or (I-5). Non-limiting examples of such materials include, but not limited to, buffers, diluents, filters, needles, syringes; carrier, package, container, vial and/or tube labels listing contents and/or instructions for use, and package inserts with instructions for use. A set of instructions will also typically be included.

A label can be on or associated with the container. A label can be on a container when letters, numbers or other characters forming the label are attached, molded or etched into the container itself; a label can be associated with a container when it is present within a receptacle or carrier that also holds the container, e.g., as a package insert. A label can be used to indicate that the contents are to be used for a specific therapeutic application. The label can also indicate directions for use of the contents, such as in the methods described herein.

In certain embodiments, the pharmaceutical compositions can be presented in a pack or dispenser device which can contain one or more unit dosage forms containing a compound of Formula (I), (I-1), (I-1a), (I-1b), (I-2), (I-2a), (I-2b), (I-3), (I-4), or (I-5). The pack can for example contain metal or plastic foil, such as a blister pack. The pack or dispenser device can be accompanied by instructions for administration. The pack or dispenser can also be accompanied with a notice associated with the container in form prescribed by a governmental agency regulating the manufacture, use, or sale of pharmaceuticals, which notice is reflective of approval by the agency of the form of the drug for human or veterinary administration. Such notice, for example, can be the labeling approved by the U.S. Food and Drug Administration for prescription drugs, or the approved product insert. Compositions containing a compound of Formula (I), (I-1), (I-1a), (I-1b), (I-2), (I-2a), (I-2b), (I-3), (I-4), or (I-5) formulated in a compatible pharmaceutical carrier can also be prepared, placed in an appropriate container, and labeled for treatment of an indicated condition.

### EXAMPLES

The symbols and conventions used in the processes, schemes, and examples described here, regardless of whether a particular abbreviation is specifically defined, are consistent with those used in the contemporary scientific literature, for example, the Journal of the American Chemical Society or the Journal of Medicinal Chemistry. Specifically, the following abbreviations may be used in the examples and throughout the specification: g (grams); mg (milligrams); mL (milliliters); μL (microliters); mM (millimolar); μM (micromolar); Hz (Hertz); MHz (megahertz); mmol (millimoles); h, hr or hrs (hours); min (minutes); MS (mass spectrometry); ESI (electrospray ionization); rt (room temperature); Rf (retention factor); TLC (thin-layer chromatography); LCMS (liquid chromatography-mass spectrometry); HPLC (high performance liquid chromatography); AcOH (acetic acid); n-BuLi (n-Butyl lithium); tBuOK (potassium tert butoxide); CDCl₃ (Chloroform-d); CH₃CN or ACN (acetonitrile); Cs₂CO₃ (cesium carbonate); DMF (N,N-Dimethylformamide); DCM (dichloromethane); DEA (diethylamine); DIPEA (diisopropyl ethylamine); DMSO (dimethyl sulfoxide); DMSO-d6 (dimethyl sulfoxide-d₆); EtOAc or EA (ethyl acetate); Et₃N (triethylamine); EtOH (ethanol); HCl (hydrochloric acid); H2SO4 (sulfuric acid); K₂CO₃ (potassium carbonate); LiOH (lithium hydroxide); MsCl (methane sulphonyl chloride); Mel (methyl iodide); MeOH (Methanol); MeOH-d4 (Methanol-d4); NaBH₄ (sodium borohydride); NaH (sodium hydride); NaHCO₃(sodium bicarbonate); NaNO2 (sodium nitrite); NaOH (sodium hydroxide); NaOMe (sodium methoxide); Na2SO4 (sodium sulphate); K3FeCN6 (potassium ferricyanide); Pd(PPh3)₄ (tetrakis(triphenylphosphine)palladium(O)); SO2 (sulfur dioxide); SO₂Cl₂ (sulfuryl chloride); T₃P (propanephosphonic acid anhydride); THE (tetrahydrofuran); TMEDA (1,2 bis(dimethylamino)ethane); Zn (zinc); ZnCN₂ (zinc cyanide).

For all the following examples, standard work-up and purification methods known to those skilled in the art can be utilized. Unless otherwise indicated, all temperatures are expressed in ° C. (degrees Celsius). All reactions are conducted at room temperature unless otherwise noted. Synthetic methodologies illustrated herein are intended to exemplify the applicable chemistry through the use of specific examples and are not indicative of the scope of the disclosure.

### Example 1: Synthesis of Select Compounds

### Scheme 1: Synthesis of (S)-1-((((1-Ethoxy-4-methyl-1-oxopentan-2-yl)carbamoyl)oxy)methyl)-3-((2-(nitrooxy)ethyl)carbamoyl)pyridin-1-ium-formate (Compound 1 Formate) and nitrate (Compound 1 Nitrate)

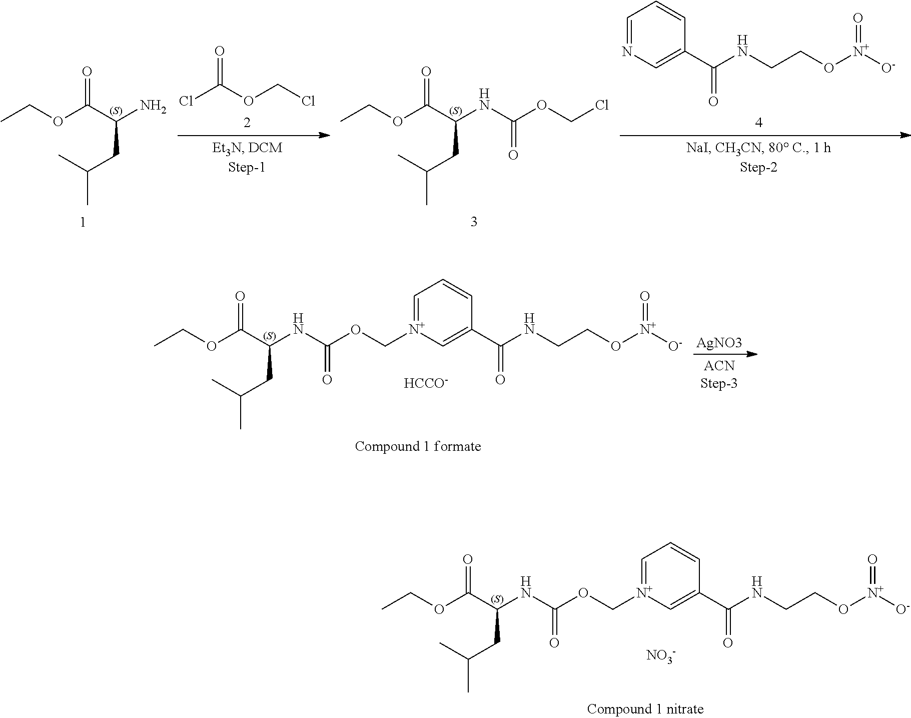

### Step-1: Synthesis of Ethyl ((chloromethoxy)carbonyl)-L-leucinate (3)

To a stirred solution of compound 1 (1.0 g, 1.0 eq., 6.28 mmol) in DCM (10 mL) were added TEA (1.28 g, 2 eq., 12.56 mmol) and chloromethyl carbonochloridate (2, 0.81 g, 1.0 eq., 6.28 mmol) at 0° C. The reaction mixture was stirred at room temperature for 16 h. After completion (monitored by TLC), the reaction mixture was quenched with water, extracted with ethyl acetate, dried over anhydrous Na₂SO₄, and evaporated under vacuum to yield the title compound, 3 (0.90 g, 60% yield) as a sticky liquid. This was used in the next step without further characterization.

### Step-2: Synthesis of (S)-1-((((1-Ethoxy-4-methyl-1-oxopentan-2-yl)carbamoyl)oxy)methyl)-3-((2-(nitrooxy)ethyl)carbamoyl)pyridin-1-ium formate (Syn. Ex. 1-formate)

To a stirred solution of compound 3 (0.9 g, 1.0 eq., 3.57 mmol) in ACN (10 mL) were added NaI (0.535 g, 1.0 eq., 3.57 mmol) and compound 4 (0.755 g, 1.0 eq., 3.57 mmol) at 0° C. The reaction mixture was stirred to 80° C. for 2 h. After completion of the reaction (monitored by TLC and LCMS), the reaction mixture was quenched with water and extracted with ethyl acetate. The combined organic layers were washed with water and brine, dried over anhydrous Na₂SO₄, and concentrated under reduced pressure. Purification was accomplished by prep HPLC (0.1% Formic Acid in water-ACN Gradient) to provide the title compound, Compound 1-formate (0.450 g, 29.60% yield) as a sticky liquid. m/z 427.20 (M⁺); ¹H NMR (400 MHz, DMSO-d₆) δ=9.61-9.54 (m, 1H), 9.50-9.44 (m, 1H), 9.34-9.27 (m, 1H), 9.05 (br d, J=8.2 Hz, 1H), 8.46-8.35 (m, 2H), 8.16 (s, 1H), 6.46 (s, 2H), 4.73-4.63 (m, 2H), 4.12-3.95 (m, 3H), 3.72 (q, J=5.4 Hz, 2H), 1.66-1.44 (m, 3H), 1.15-1.09 (m, 3H), 0.88-0.77 (m, 6H).

### Step-3: Synthesis of (S)-1-((((1-Ethoxy-4-methyl-1-oxopentan-2-yl)carbamoyl)oxy)methyl)-3-((2-(nitrooxy)ethyl)carbamoyl)pyridin-1-ium nitrate (Syn. Ex. 1—nitrate)

To a stirred solution of Compound 1-formate (0.100 g, 1.0 eq., 0.213 mmol) in ACN (2 mL) was added AgNO₃ (0.037 g, 1.0 eq., 0.213 mmol) at 0° C. The reaction was stirred at RT for 2 h. After completion of the reaction, the reaction mixture was filtered through Buchner funnel. The precipitate was washed with diethyl ether and dried under high vacuum to get the title compound, Compound 1-Nitrate (0.020 g, 19.23% yield) as a white solid. m/z 427.20 (M⁺); ¹H NMR (400 MHz, DMSO-d₆) δ=9.56 (s, 1H), 9.49-9.40 (m, 1H), 9.33-9.27 (m, 1H), 9.11-9.02 (m, 1H), 8.46-8.35 (m, 2H), 6.46 (s 2H), 4.72-4.65 (m, 2H), 4.12-3.96 (m, 3H), 3.78-3.69 (m, 2H), 1.67-1.41 (m, 3H), 1.16-1.09 (m, 3H), 0.87-0.80 (m, 6H).

### Scheme 2: Synthesis of 1-((5-Methyl-2-oxo-1,3-dioxol-4-yl)methyl)-3-((2-(nitrooxy)ethyl)carbamoyl)pyridin-1-ium formate (Compound 2-formate)

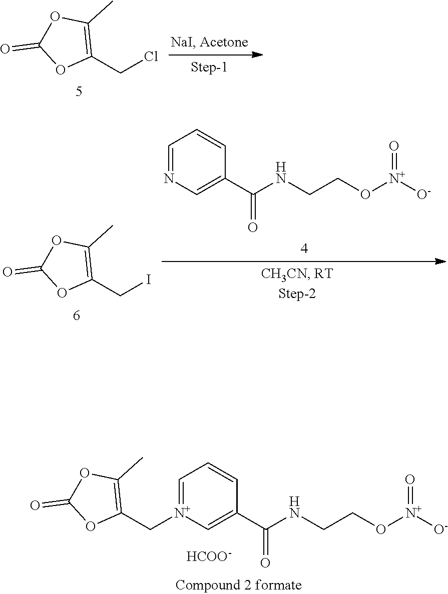

### Step-1: Synthesis of 4-(Iodomethyl)-5-methyl-1, 3-dioxol-2-one (6)

To a stirred solution of 4-(chloromethyl)-5-methyl-1, 3-dioxol-2-one (5, 1.5 g, 1.0 eq., 10.0 mmol) in acetone (15 mL) at 25° C. was added NaI (2.3 g, 1.5 eq., 15.0 mmol). The reaction mixture was stirred at rt for 3 h. After completion of the reaction (monitored by TLC), the reaction mixture was quenched with water and extracted with ethyl acetate. The combined organic layers were washed with water and brine, dried over anhydrous Na₂SO₄, and concentrated under reduced pressure. Purification was accomplished by combi-flash chromatography (using 0-7% ethyl acetate in heptane as an eluent) to provide the title compound, 6 (0.900 g, 36.29% yield) as a sticky liquid. This was used in the next step without further characterization.

### Step-2: Synthesis of 1-((5-Methyl-2-oxo-1,3-dioxol-4-yl)methyl)-3-((2-(nitrooxy)ethyl)carbamoyl)pyridin-1-ium-formate (Compound 2-formate)

To a stirred solution of compound 6 (0.70 g, 1.0 eq., 2.91 mmol) in ACN (5 mL) was added compound 4 (0.615 g, 1.0 eq., 2.91 mmol) at 0° C. The reaction mixture was heated to 85° C. for 1.5 h. After completion of the reaction (monitored by TLC and LCMS), acetonitrile was removed under vacuum to afford the crude which was purified by prep HPLC twice (0.1% Formic Acid in water-ACN gradient)) to provide the title compound, Compound 2-formate (0.050 g, 5.29% yield) as a white solid. m/z 324.1 (M⁺); ¹H NMR (400 MHz, DMSO-d₆) δ=9.00 (d, J=1.6 Hz, 1H), 8.96-8.89 (m, 1H), 8.77-8.67 (m, 1H), 8.19 (td, J=1.8, 8.0 Hz, 1H), 7.60-7.46 (m, 1H), 4.70-4.63 (m, 2H), 3.65 (q, J=5.5 Hz, 2H), 3.33 (s, 3H), 2.56-2.52 (m, 2H).

### Scheme 3: Synthesis of (S)-1-((((1-Methoxy-1-oxopropan-2-yl)carbamoyl)oxy)methyl)-3-((2-(nitrooxy)ethyl)carbamoyl)pyridin-1-ium-formate (Syn. Ex. 3-formate)

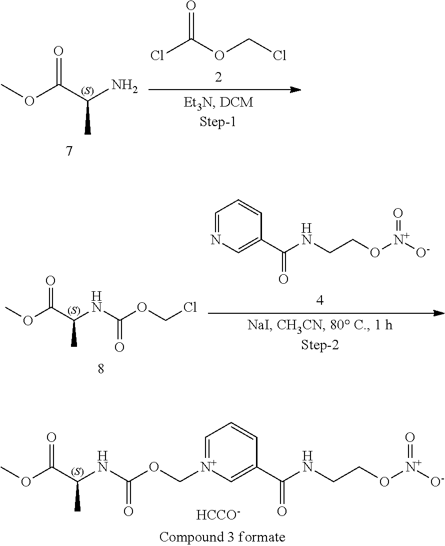

Step-1: Synthesis of Methyl ((chloromethoxy)carbonyl)-L-alaninate (8) To a stirred solution of compound 7 (0.50 g, 1.0 eq., 4.85 mmol) in DCM (10 mL) were added TEA (0.98 g, 2 eq., 9.70 mmol) and chloromethyl carbonochloridate (2, 0.62 g, 1.0 eq., 4.85 mmol) at 0° C. The reaction mixture was stirred at rt for 4 h. After completion (monitored by TLC), the reaction mixture was quenched with water, extracted with ethyl acetate, the combined organic layers were washed with aq. NaHCO₃ solution, dried over anhydrous Na₂SO₄, and evaporated under vacuum to yield the title compound, 8 (0.420 g, 36.52% yield) as a sticky liquid. This was used in the next step without further characterization.

### Step-2: Synthesis of (S)-1-((((1-Methoxy-1-oxopropan-2-yl)carbamoyl)oxy)methyl)-3-((2-(nitrooxy)ethyl)carbamoyl)pyridin-1-ium-formate (Compound 3-formate)

To a stirred solution of compound 8 (0.420 g, 1.0 eq., 2.14 mmol) in ACN (5 mL) were added NaI (0.325 g, 1.0 eq., 2.144 mmol) and compound 4 (0.453 g, 1.0 eq., 2.14 mmol) at 0° C. The reaction mixture was stirred to 80° C. for 1 h. After completion of the reaction (monitored by TLC and LCMS), acetonitrile was removed under vacuum to obtain the crude. The crude was purified by prep HPLC (0.1% Formic Acid in water-ACN Gradient) twice to provide the title compound, Compound 3-formate. (0.020 g, 2.23% yield) as a white solid. m/z 371.37 (M⁺); ¹H NMR (400 MHz, DMSO-d₆) δ=9.58 (s, 1H), 9.52 (br t, J=5.5 Hz, 1H), 9.32-9.26 (m, 1H), 9.11-9.02 (m, 1H), 8.49-8.44 (m, 1H), 8.40-8.34 (m, 1H), 6.52-6.40 (m, 2H), 4.73-4.67 (m, 2H), 4.16-4.07 (m, 1H), 3.72 (q, J=5.4 Hz, 2H), 3.61 (s, 3H), 1.28 (d, J=7.3 Hz, 3H).

Additional compounds that can be prepared following the procedures described herein include:

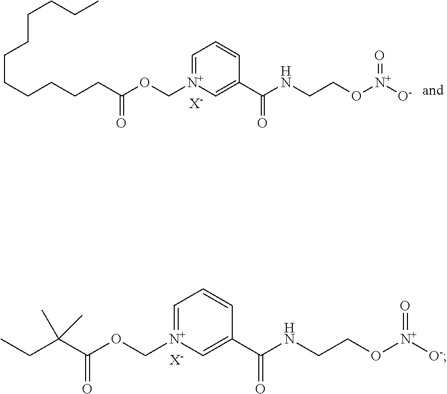

wherein X⁻ indicates a counter ion.

### Example 2: Intestinal Microsomal Stability

As used in this and the following examples, the following compound identifiers will be used:

| Compound ID | Structure |
|---|---|
| Nitrate salt of compound 8 from PCT Application WO 2020/190890 (Compound 8*) | 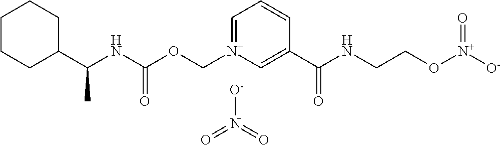 |
| Compound 10 | 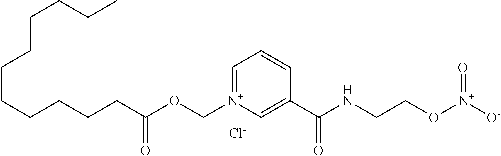 [SMILES: CCCCCCCCCCCC(=O)OC[n+]1cccc(C(=O)NCCO[N+](=O)[O-])c1 ; [Cl-]] |
| Compound 11 | 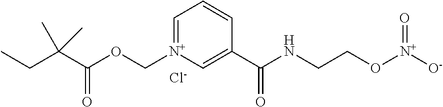 [SMILES: CCC(C)(C)C(=O)OC[n+]1cccc(C(=O)NCCO[N+](=O)[O-])c1 ; [Cl-]] |
| Compound 12 | 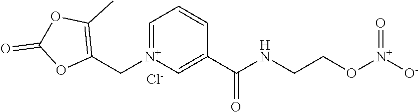 [SMILES: Cc1oc(=O)oc1C[n+]1cccc(C(=O)NCCO[N+](=O)[O-])c1 ; [Cl-]] |
| Compound 13 | 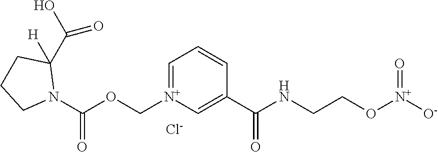 [SMILES: O=C(NCCO[N+](=O)[O-])c1ccc[n+](COC(=O)N2CCCC2C(=O)O)c1 ; [Cl-]] |

Each test compound and assay control (final concentration of 1 CM) were incubated with intestinal microsomes (final protein concentration of 1 mg/mL) from rats, dogs or humans at 37° C. in the presence of NADPH for 0.5, 5, 10, 15, 30 and 60 minutes. The reaction was terminated by adding ice-cold acetonitrile containing internal standard (cetirizine/tolbutamide/telmisartan) at each time point following incubation. Samples were then centrifuged at 4000 rpm for 10 minutes and the supernatant was analyzed by LC-MS/MS. The in vitro half-life (t_{1/2}) and intrinsic clearance (CLᵢₙₜ) were calculated for the test compounds and the assay control. The results are provided in Table 1 below.

TABLE 1
Half-life and intrinsic clearance for compounds tested in intestinal microsomes.

| | Rat | | Dog | | Human | |
|---|---|---|---|---|---|---|
| | | CLᵢₙₜ | | CLᵢₙₜ | | CLᵢₙₜ |
| Compound | | (μL/min/mg | | (μL/min/mg | | (μL/min/mg |
| ID | t_{1/2} (min) | protein) | t_{1/2} (min) | protein) | t_{1/2} (min) | protein) |
| Nitrate Salt | >60 | <6 | >60 | <6 | >60 | 9 |
| of Compound | | | | | | |
| 8 (WO 2020/190890) | | | | | | |
| 10 | 26 | 27 | 47 | 15 | 24 | 29 |
| 13 | >60 | <6 | >60 | <6 | >60 | <6 |
| 11 | >60 | 8 | >60 | 9 | >60 | <6 |

These data demonstrate that compound 10 has high clearance and rapidly release nicorandil in rat, dog and human intestinal microsomes, compared to the nitrate salt of compound 8 of WO 2020/190890 and compounds 11 and 13.

### Example 3: Plasma Stability

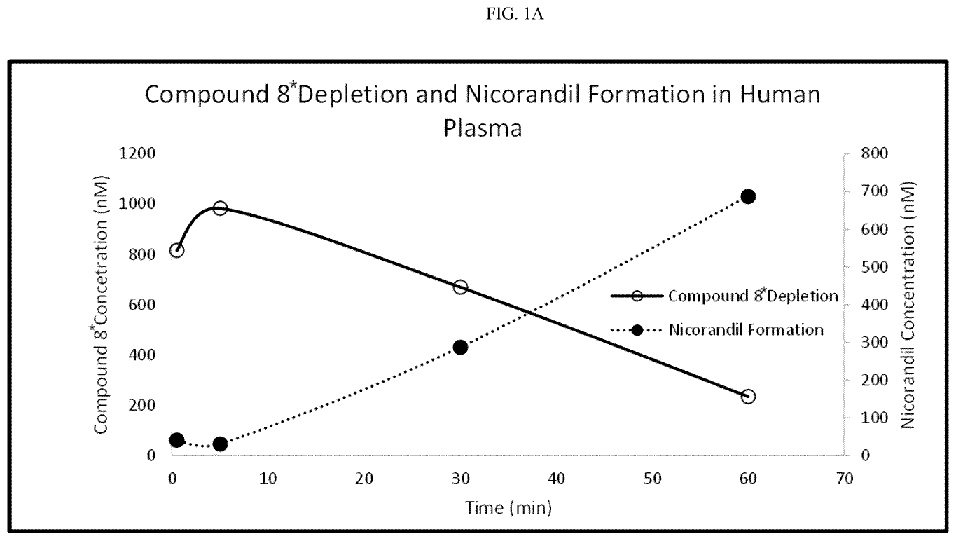

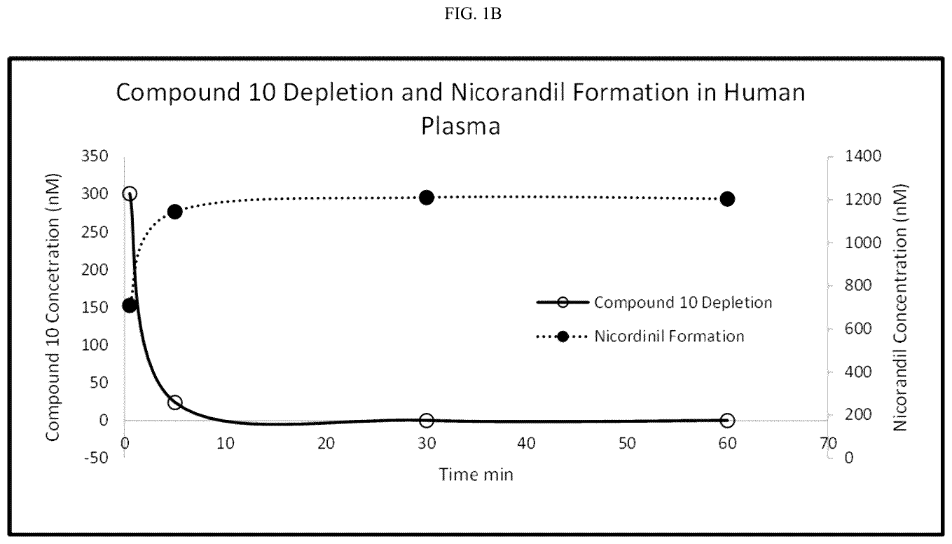

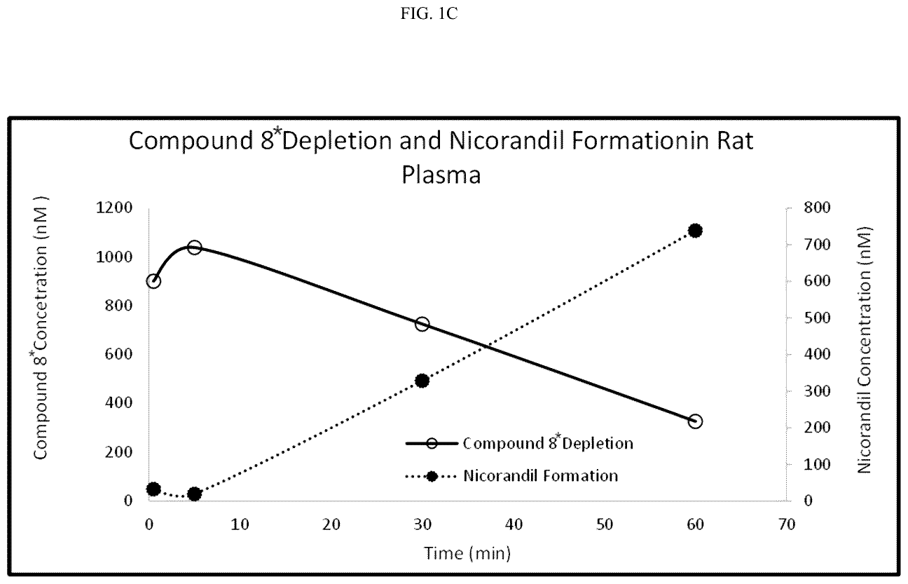

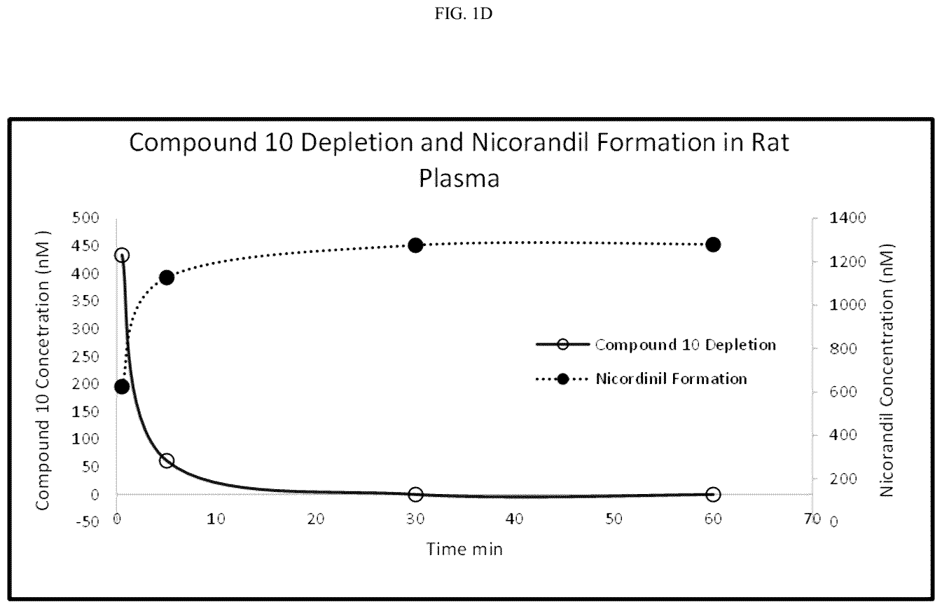

Each test compound and assay control (final concentration of 1 μM) were incubated with human plasma or rat plasma at 37° C. for 0.5, 5, 30 and 60 minutes. The reaction was quenched with ice-cold acetonitrile containing internal standard (cetirizine/tolbutamide/telmisartan) following incubation. Samples were then centrifuged at 4000 rpm at 4 (C for 10 minutes and the supernatant was analyzed by LC-MS/MS. The parent compound remaining (% PCR) and half-life (t_{1/2}) were calculated for the test compounds and the assay control. The results are provided in Tables 2 and 3 below and in FIGS. 1A-1D.

TABLE 2
% PCR, half-life, nicorandil concentration and % formation
of nicorandil for compounds incubated in human plasma.

| | % PCR | | Concentration | % |
|---|---|---|---|---|
| Compound | (at 60 | t_{1/2} | (nM) of nicorandil | formation of |
| ID | minutes) | (min) | (at 60 minutes) | nicorandil |
| Propantheline | 9.2 | 21.3 | N/A | N/A |
| Bromide | | | | |
| 10 | 0 | 1.23 | 1204 | 100 |
| Nitrate | 28.7 | 31.2 | 688 | 75 |
| Salt of | | | | |
| Compound 8 | | | | |
| (WO | | | | |
| 2020/190890) | | | | |
| Nicorandil | 109 | >60 | N/A | N/A |

TABLE 3
% PCR, half-life, nicorandil concentration and % formation
of nicorandil for compounds incubated in rat plasma.

| | % PCR | | Concentration | % |
|---|---|---|---|---|
| Compound | (at 60 | t_{1/2} | (nM) of nicorandil | formation of |
| ID | minutes) | (min) | (at 60 minutes) | nicorandil |
| Enalapril | 11.2 | 18.9 | N/A | N/A |
| 10 | 0 | 1.59 | 1279 | 100 |
| Nitrate | 36.2 | 38.2 | 739 | 69 |
| Salt of | | | | |
| Compound 8 | | | | |
| (WO | | | | |
| 2020/190890) | | | | |
| Nicorandil | 114 | > 60 | N/A | N/A |

As shown by the data, compound 10 is rapidly released in human and rat plasma compared to the nitrate salt of compound 8 of WO 2020/190890 (FIGS. 1A-1D; Table 2-3).

### Example 4: Liver Microsomal Stability

Each test compound and assay control (final concentration of 1 μM) were incubated with liver microsomes (final protein concentration of 0.5 mg/mL) from rats, dogs or humans at 37° C. in the presence of NADPH for 0.5, 5, 10, 15, 30 and 60 min. The reaction was terminated by adding ice-cold acetonitrile containing internal standard at each time point following incubation. The samples were then centrifuged at 4000 rpm for 10 minutes and the supernatant was analyzed by LC-MS/MS. The predicted hepatic clearance and the hepatic extraction ratio were calculated for the test compounds and the assay control. The results are provided in Table 4 below.

TABLE 4
Hepatic clearance/hepatic extraction ratios for test compounds.

| | Predicted Hepatic CL (mL/min/kg)/ | | |
|---|---|---|---|
| Compound | Hepatic Extraction Ratio | | |
| ID | Rat | Dog | Human |
| Nitrate Salt of | >49/>0.89 | 26/0.84 | 15/0.71 |
| Compound 8 (WO | | | |
| 2020/190890) | | | |
| 10 | 46/0.84 | 4/1.0 | 19/0.90 |

These data demonstrate that compound 10 was unstable in the liver microsomes of rat, dog and human compared to the nitrate salt of compound 8 of WO 2020/190890.

### Example 5: Pharmacokinetic Study in Rats

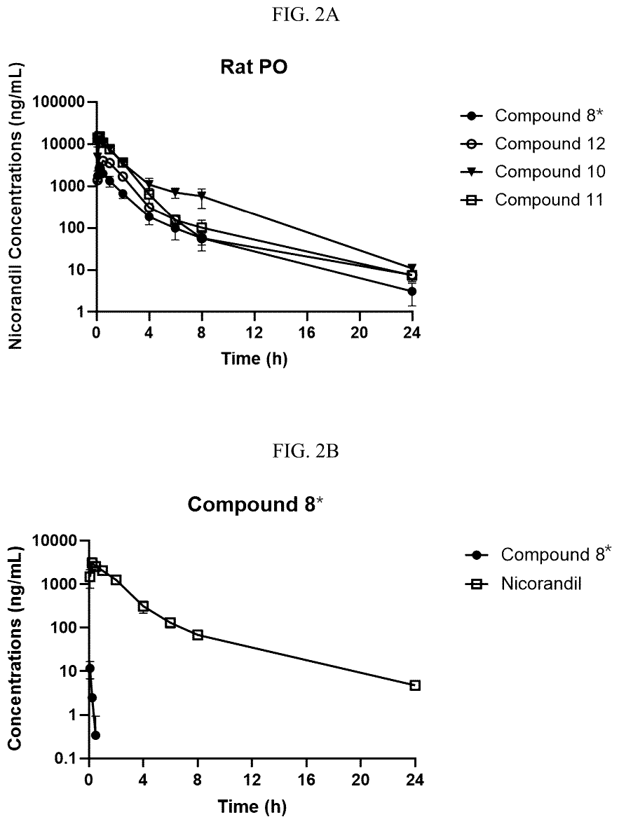

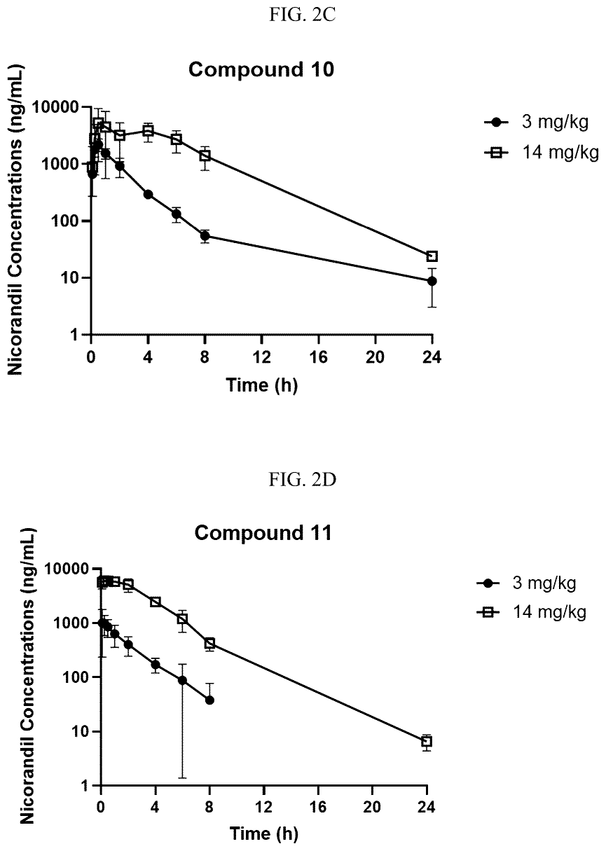

Jugular vein cannulated male Sprague-Dawley rats of 8-12 weeks of age were orally administered a single dose (3 mg/kg equivalent dose of nicorandil) of each test compound formulated either in citrate buffer pH 4.5 or in 0.25% (w/v) sodium carboxymethyl cellulose (sodium CMC) in water. Following oral administration, approximately 150 μL of blood was collected from each animal (n=3 for each compound) through jugular vein catheter in Eppendorf tubes containing K₂EDTA solution at 0.083, 0.25, 0.5, 1, 2, 4, 6, 8 and 24 hours after dosing, and then transferred immediately to micro-centrifuge tubes containing paraoxon. Plasma was harvested by centrifugation of blood at 10,000 rpm for 10 minutes. Plasma was transferred to micro-centrifuge tubes containing stabilizer (20% acetic acid in water). Bioanalysis was performed for quantification of nicorandil using the fit-for-purpose LC-MS/MS method. The dose formulations were analyzed for accuracy by HPLC for pro-drug recovery. Pharmacokinetic parameters: Cₘₐₓ, Tₘₐₓ, AUC and t₁/2 were estimated using PHOENIX(R) WinNonlin software (Ver: 8.3). Oral bioavailability (F (%)) was also calculated. The same process was performed with a 14 mg/kg dose for compounds 10 and 11. Data are presented in Tables 5-8 below and in FIGS. 2A-2D.

TABLE 5
Pharmacokinetic parameters for test compounds in citrate buffer.

| | Cₘₐₓ | Median Tₘₐₓ | AUCₗₐₛₜ |
|---|---|---|---|
| Compound ID | (ng/mL) | (h) | (ng * h/mL) |
| Nitrate Salt of | 2934 | 0.25 | 4679 |
| Compound 8 (WO | | | |
| 2020/190890) | | | |
| 12 | 4065 | 0.5 | 8893 |
| 10 | 12,446 | 0.25 | 26,838 |
| 11 | 15,223 | 0.25 | 22,632 |

TABLE 6
Pharmacokinetic parameters for test compound 8*
and nicorandil in a 0.25% sodium CMC formulation.

| Compound | Cₘₐₓ | Tₘₐₓ | AUCₗₐₛₜ | t_{1/2} | F |
|---|---|---|---|---|---|
| ID | (ng/mL) | (h) | (ng * h/mL) | (h) | (%) |
| Nitrate | 11.7 | 0.08 | 1.8 | NC | NC |
| Salt of | | | | | |
| Compound 8 (WO | | | | | |
| 2020/190890) | | | | | |
| Nicorandil | 3083 | 0.25 | 6729 | 3.61 | 82 |

TABLE 7
Pharmacokinetic parameters for test compound
10 in a 0.25% sodium CMC formulation.

| Dose | Cₘₐₓ | Tₘₐₓ | AUCₗₐₛₜ | t_{1/2} | F |
|---|---|---|---|---|---|
| (mg/kg) | (ng/mL) | (h) | (ng * h/mL) | (h) | (%) |
| 3 | 2226 | 0.5 | 5513 | 4.07 | 67 |
| 14 | 6297 | 4.0 | 36,526 | 2.74 | 97 |

TABLE 8
Pharmacokinetic parameters for test compound
11 in a 0.25% sodium CMC formulation.

| Dose | Cₘₐₓ | Tₘₐₓ | AUCₗₐₛₜ | t_{1/2} | F |
|---|---|---|---|---|---|
| (mg/kg) | (ng/mL) | (h) | (ng * h/mL) | (h) | (%) |
| 3 | 1171 | 0.25 | 2289 | 1.75 | 28 |
| 14 | 6245 | 0.25 | 27422 | 2.52 | 72 |

As shown by the data and Figures, after oral dosing, compounds 10-12 released higher levels of nicorandil compared to the nitrate salt of compound 8 of WO 2020/190890 (FIG. 2A; Table 5). Compound 8* was shown to release nicorandil in vivo (FIG. 2B; Table 6) as were compounds 10 (FIG. 2C; Table 7) and 11 (FIG. 2D; Table 8). The increase in AUC from 3 mg/kg to 14 mg/kg for both compounds 10 and 11 was more than dose proportional.

The data obtained in Examples 2-5 demonstrate that compound 10, a prodrug of nicorandil, has the potential to mitigate gut toxicities associated with nicorandil. Compound 10 is unstable in rat, dog and human intestinal microsomes. These data indicate that compound 10 should deliver nicorandil only after it has been absorbed into the gut. Compound 10 rapidly releases nicorandil in human plasma. Amounts of compound 10 that get absorbed and into circulation should rapidly release nicorandil. This is also true for rat plasma which is favorable for pharmacology models. Compound 10 rapidly releases nicorandil in human liver microsomes. Amounts of compound 10 that get absorbed should be metabolized to nicorandil in the liver. This is also true in rat and dog liver microsomes which is favorable for pharmacology models. Oral bioavailability of nicorandil after oral dosing of compound 10 is high based on pharmacokinetic data using two different formulations and two different doses.

The specific and non-limiting examples are to be construed as merely illustrative, and do not limit the present disclosure in any way whatsoever. Without further elaboration, it is believed that one skilled in the art can, based on the description herein, utilize the present disclosure to its fullest extent. All publications cited herein are hereby incorporated by reference in their entirety. Where reference is made to a URL or other such identifier or address, it is understood that such identifiers can change and particular information on the internet can come and go, but equivalent information can be found by searching the internet. Reference thereto evidences the availability and public dissemination of such information.

It is understood that the examples and embodiments described herein are for illustrative purposes only and that various modifications or changes in light thereof will be suggested to persons skilled in the art and are to be included within the spirit and purview of this application and scope of the appended claims. All publications, patents, and patent applications cited herein are hereby incorporated by reference in their entirety for all purposes.

1. A compound according to formula (I): 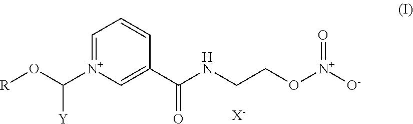
or a stereoisomer thereof;
wherein X⁻ indicates one or more counter ions;
Y is H or C₁-C₄ alkyl;
R is selected from 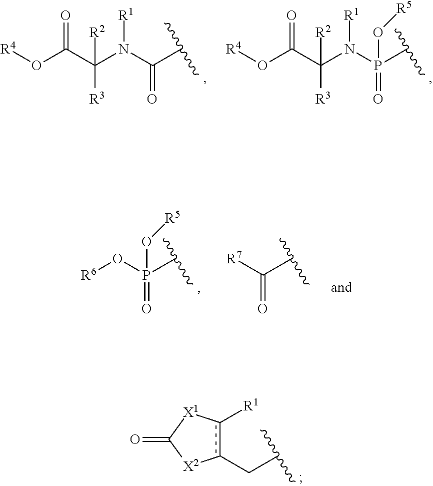
R¹ is H or C₁-C₄ alkyl;
R² is H or C₁-C₄ alkyl;
R³ is H or unsubstituted or substituted C₁-C₄ alkyl; or R¹ and R³ are joined together to form a 4-7 membered substituted or unsubstituted heterocycloalkyl;
R⁴ is H or C₁-C₈ alkyl;
R⁵ and R⁶ are independently selected from hydrogen, C₁-C₈ alkyl, a 6-10 membered substituted or unsubstituted aryl, and a 6-10 membered substituted or unsubstituted heteroaryl;
R⁷ is H or C₁-C₁₂ alkyl;
X¹ and X² are independently selected from NH, O, and S; and [symbol]
is a single or double bond;
wherein when Y is H, R⁷ is not —CH(CH₃)₂, —CH₂CH(CH₃)₂, —C(CH₃)₃, or —CH₂C(CH₃)₃.

2. The compound of claim 1, according to Formula (I-1): 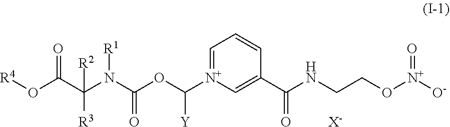
or a stereoisomer thereof.

3. The compound of claim 2, according to Formula I-1a: 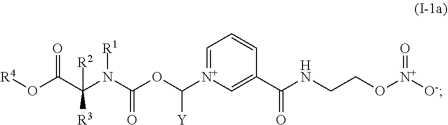
or a stereoisomer thereof.

4. The compound of claim 2, according to Formula I-1b: 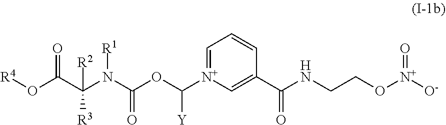
or a stereoisomer thereof.

5. The compound of claim 1, according to Formula (I-2): 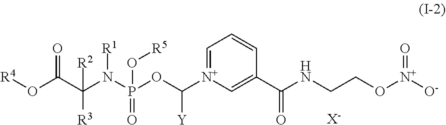
or a stereoisomer thereof.

6. The compound of claim 5, according to Formula (I-2a): 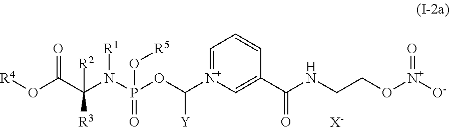
or a stereoisomer thereof.

7. The compound of claim 5, according to Formula (I-2b): 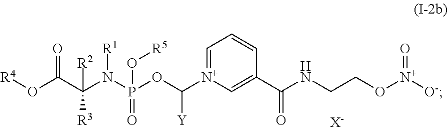
or a stereoisomer thereof.

8. The compound of claim 1, wherein:
R³ is H or unsubstituted or substituted C₁-C₄ alkyl.

9. The compound of claim 1, wherein R³ is substituted C₁-C₄ alkyl and the substitutions are selected from hydroxyl, heteroalkyl, aryl, heteroaryl, —COOH, —CONH₂, and —NH₂.

10. The compound of claim 1, wherein R³ is selected from H, —CH₃, —(CH₂)₃—NH—C(NH₂)═NH, —CH₂—CONH₂, —CH₂—COOH, —CH₂—SH, —(CH₂)₂—CONH₂, —(CH₂)₂—COOH, —CH₂-1H-imidazole, —CH(CH₃)CH₂—CH₃, —CH₂—CH—(CH₃)₂, —(CH₂)₄—NH₂,
—(CH₂)₂—S—CH₃, —CH₂-Ph, —CH₂—OH, —CH(OH)—CH₃, —CH₂-1H-indole, —CH₂-Ph-OH, and —CH—(CH₃)₂.

11. The compound of claim 10, wherein R³ is selected from —CH₃, —CH₂—OH, —CH₂-Ph, —CH₂—CONH₂, and CH₂—C(H)—(CH₃)₂.

12. The compound of claim 2, wherein R³ is selected from —CH₃, —CH₂—OH, —CH₂-Ph, —CH₂—CONH₂, and CH₂—C(H)—(CH₃)₂.

13. The compound of claim 1, wherein R¹ is hydrogen.

14. The compound of claim 1, wherein R¹ and R³ are joined together to form a pyrrolidine ring.

15. The compound of claim 1, wherein R⁵ is a 6-10 membered substituted or unsubstituted aryl.

16. The compound of claim 1, wherein R² is H.

17. The compound of claim 1, wherein R⁴ is H.

18. The compound of claim 1, wherein R⁴ is C₁-C₈ alkyl.

19. The compound of claim 18, wherein R⁴ is H, —CH₃, —CH₂—CH₃, or —CH(CH₃)₂.

20. The compound of claim 1, of Formula (I-3): 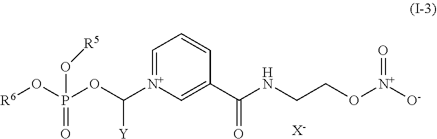
or a stereoisomer thereof.

21. The compound of claim 20, wherein R⁵ and R⁶ are independently selected from hydrogen and C₁-C₈ alkyl.

22. The compound of claim 1, according to Formula (I-4): 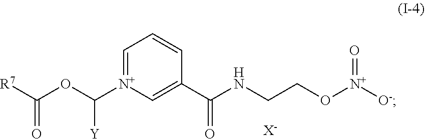
or a stereoisomer thereof.

23. The compound of claim 22, wherein R⁷ is C₁-C₁₂ alkyl.

24. The compound of claim 22, wherein R⁷ is C₁-C₂ alkyl.

25. The compound of claim 22, wherein R⁷ is C₆-C₁₂ alkyl.

26. The compound of claim 1, according to Formula (I-5): 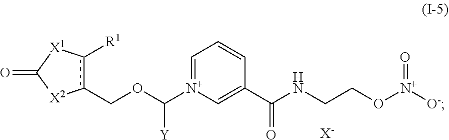
or a stereoisomer thereof.

27. The compound of claim 26, wherein X¹ and X² are both H.

28. The compound of claim 26, wherein R¹ is C₁-C₄ alkyl.

29. The compound of claim 1, wherein Y is H.

30. The compound of claim 2, of the formula: 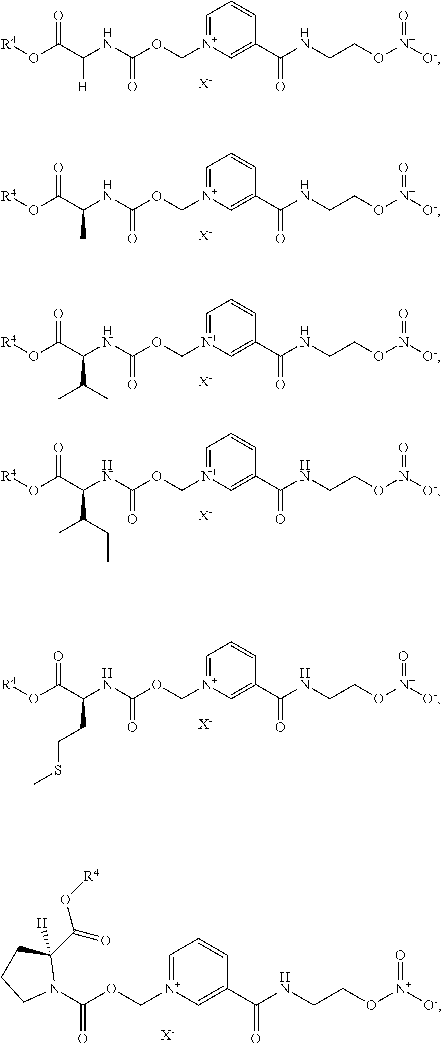 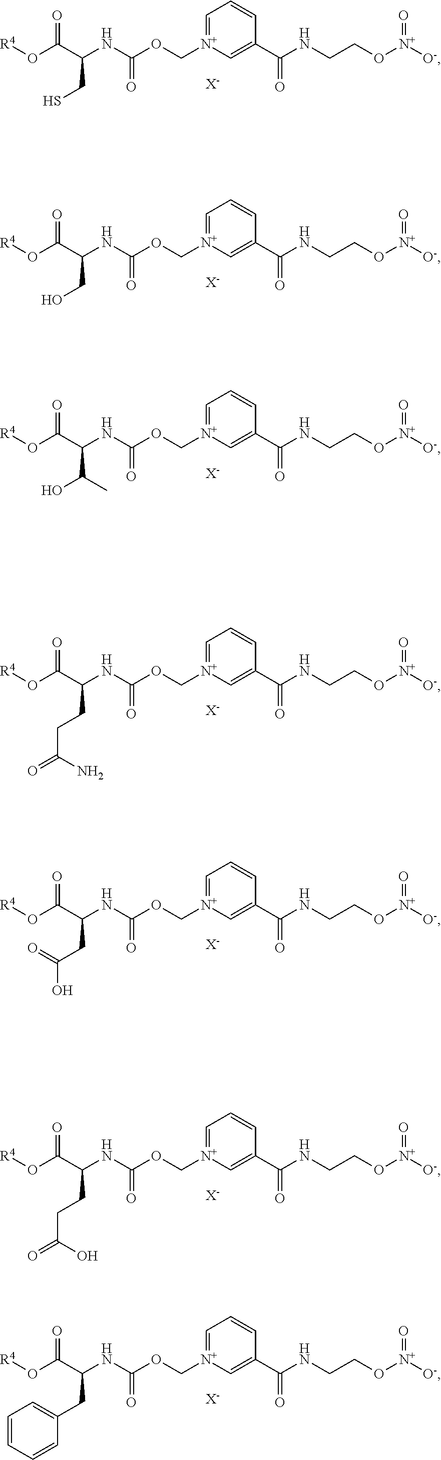 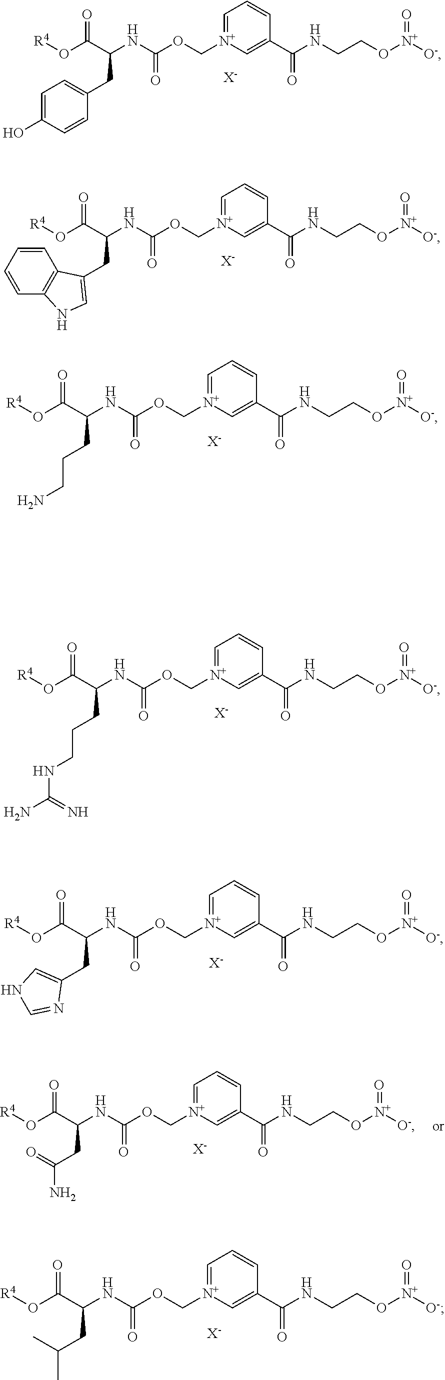
or a stereoisomer thereof.

31. The compound of claim 1, selected from

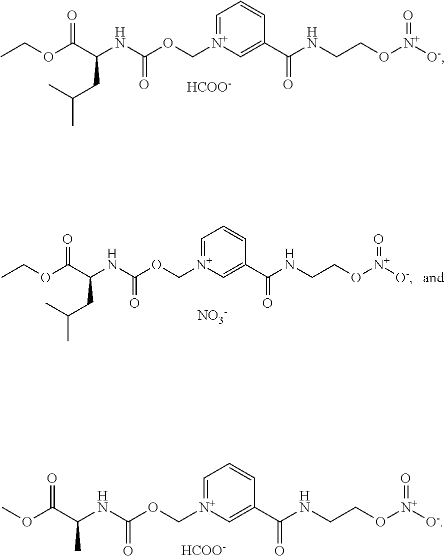

32. The compound of claim 1 selected from: 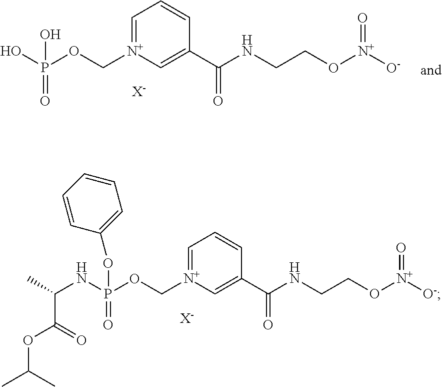
or a stereoisomer thereof.

33. The compound of claim 1, selected from the group consisting of:

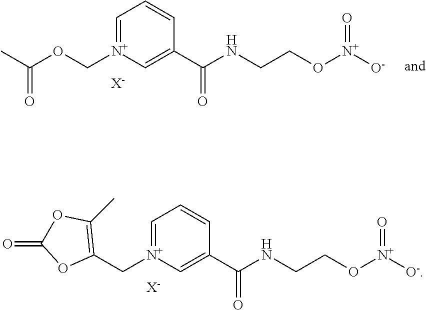

34. The compound of claim 33, of the formula:

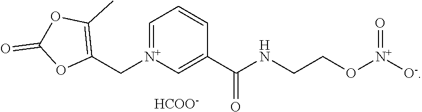

35. The compound of claim 1, selected from the group consisting of:

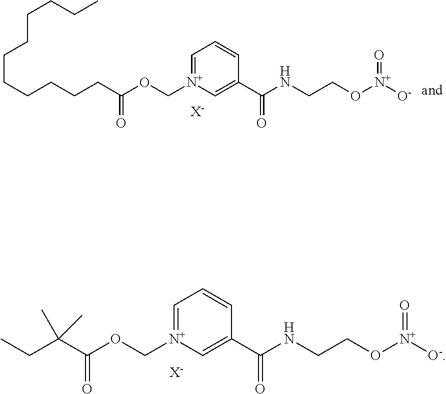

36. The compound of claim 1, wherein X⁻ is selected from F⁻, Cl⁻, Br⁻, I⁻, MeSO₂O⁻, acetate, carbonate, chromate, citrate, fumarate, lactate, malonate, nitrate, phosphate, tartrate, trifluoroacetate, succinate, formate and sulfate.

37. A pharmaceutical composition comprising the compound of claim 1 and one or more pharmaceutically acceptable carriers, excipients, or diluents.

38. A method for treating a disease or a condition comprising the step of administering an effective amount of the compound of claim 1 or a pharmaceutical composition thereof to a subject in need thereof.

39. The method of claim 38, wherein the disease or condition is acute kidney disease, chronic kidney disease, diabetic nephropathy, IgA nephropathy, acute kidney failure, acute tubular necrosis, transplant related ischemia, or acute kidney injury (AKI).

40. The method of claim 39, wherein the AKI is drug-induced AKI.

41. The method of claim 40, wherein the drug-induced AKI is chemotherapy-induced AKI or immunotherapy-induced AKI.

42. The method of claim 41, wherein the AKI is cisplatin induced AKI, aminoglycosides induced AKI, tenofovir induced AKI, contrast induced nephropathy, or PD-1/PDL-1 inhibitor-induced AKI.

43. The method of claim 42, wherein the PD-1/PDL-1 inhibitor-induced AKI is pembrolizumab (Keytruda)-induced AKI, durvalumab-induced AKI, atezolizumab-induced AKI, avelumab-induced AKI, nivolumab-induced AKI, durvalumab-induced AKI, cemiplimab-induced AKI, dostarlimab-induced AKI, and retifanlimab-induced AKI.

44. The method of claim 39, wherein the AKI is AKI after surgery for partial nephrectomy, AKI post kidney transplant, AKI in patients with dialysis, AKI after cardiac surgery, AKI in patients inside ICU, or AKI caused by cirrhosis or sepsis and AKI caused by hepatorenal syndrome.

45. The method of claim 39, wherein the disease or condition is acute kidney failure.

46. The method of claim 39, wherein the disease or condition is chronic kidney disease.

47. (canceled)

48. (canceled)
